SEQUENCE LISTING (91 sequences)

SEQ ID NO: 1 - protein, 5 aa, synthetic construct
GSGGS

SEQ ID NO: 2 - protein, 4 aa, synthetic construct
GGGS

SEQ ID NO: 3 - protein, 5 aa, synthetic construct
GGGGS

SEQ ID NO: 4 - protein, 4 aa, synthetic construct
GGSG

SEQ ID NO: 5 - protein, 5 aa, synthetic construct
GGSGG

SEQ ID NO: 6 - protein, 5 aa, synthetic construct
GSGSG

SEQ ID NO: 7 - protein, 5 aa, synthetic construct
GSGGG

SEQ ID NO: 8 - protein, 5 aa, synthetic construct
GGGSG

SEQ ID NO: 9 - protein, 5 aa, synthetic construct
GSSSG

SEQ ID NO: 10 - protein, 5 aa, synthetic construct
GGGGS

SEQ ID NO: 11 - protein, 15 aa, synthetic construct
GGGGSGGGGSGGGGS

SEQ ID NO: 12 - protein, 45 aa, synthetic construct
GGTGGCGGTGGCTCGGGCGGTGGTGGGTCGGGTGGCGGCGGATCT

SEQ ID NO: 13 - protein, 10 aa, synthetic construct
KLTVLRQPKA

SEQ ID NO: 14 - protein, 8 aa, synthetic construct
VAPGKTAR

SEQ ID NO: 15 - protein, 10 aa, synthetic construct
PTIRRYYYYG

SEQ ID NO: 16 - protein, 9 aa, synthetic construct
NHKPSNTKV

SEQ ID NO: 17 - protein, 8 aa, synthetic construct
VSSASTKG

SEQ ID NO: 18 - protein, 10 aa, synthetic construct
PTIRRYYYYG

SEQ ID NO: 19 - protein, 9 aa, synthetic construct
NHKPSNTKV

SEQ ID NO: 20 - protein, 8 aa, synthetic construct
VSSASTKG

SEQ ID NO: 21 - protein, 120 aa, synthetic construct
EVQLVESGGGGGLGLVKPGGSLRLSCAASGFTFRSYSMNWVRQAPGKGLEWVSSIISSSSYIYADSVKGRFTISRDNAKNSLYLQMNSLRAEDTALYYCARDQNAMDVWGGTTVTVTVSS

SEQ ID NO: 22 - protein, 218 aa, synthetic construct
SYELTQPPSVSVSVSVAPGKTARITCAGNNIGSKSVHWYQQQQAPVLVIYYDSDRPSGIPERFSGSNSGNTATLTISRVEAGDEADYYCQVWDSGSDHPWWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 224 aa, synthetic construct
QVQLQQWGAGLLKPSETLSLTCAVYGGSFSGYYWTWIRQSPGKGLEWIGEINHSGSTTYNPSLKSRVTISVDTSKNQFSLKLNSVTAADTAVYYCARGYGDTPTIRRYYYYGMDVWGQGTTVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKC

SEQ ID NO: 24 - protein, 220 aa, synthetic construct
DIVMTQSPLSLPVTLGQPASISCRSSQSLVHSDGNTYLNWFQQRPGQSPRRLIYKVSDRDSGVPDRFSGSGSGTDFTLKISRVEAEDVGLYYCMQGTHWPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 8 aa, synthetic construct
GFSLTSYG

SEQ ID NO: 26 - protein, 7 aa, synthetic construct
IWRGGGT

SEQ ID NO: 27 - protein, 13 aa, synthetic construct
ARSRYDEEESMNY

SEQ ID NO: 28 - protein, 6 aa, synthetic construct
GNIHSY

SEQ ID NO: 29 - protein, 4 aa, synthetic construct
NAKT

SEQ ID NO: 30 - protein, 9 aa, synthetic construct
QHFWNTPPT

SEQ ID NO: 31 - protein, 119 aa, synthetic construct
QVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSS

SEQ ID NO: 32 - protein, 117 aa, synthetic construct
QLVQSGAEVVKPGASVKVSCKASGYTFTSYWMHWVKQAPGQGLEWIGAVSPGNSDTSYNEKFKGKATLTVDKSASTAYMELSSLRSEDTAVYYCTRSRYGNNALDYWGQGTLVTVSS

SEQ ID NO: 33 - DNA, 357 bp, synthetic construct
  source 1..357: mol_type = other DNA
caggtccagttgcaacagtctggccctggtctggtccaacctagtcaatctttgtccattacctgcactgtgtctggtttttctctcacgtcttatggaattcattgggttagacaaagccctgggaaagggttggagtggttgggtgtgatctggcgcggaggtggtactgacagcaacgcagcgtttatgtctcggctttccataaccaaagacaacagcaagtctcaggtgttcttcaagatgaattccctccaggccgacgatacggcgatctattattgtgctcgctccagatacgacgaggaagaaagcatgaactattggggacaggggacctcagtaacagtgtcaagc

SEQ ID NO: 34 - DNA, 351 bp, synthetic construct
  source 1..351: mol_type = other DNA
caactggttcagtcaggtgctgaagttgtaaaaccaggcgcttctgttaaagtatcctgtaaagctagcggatatacatttaccagttactggatgcactgggtaaagcaggctccaggacaaggactggagtggatcggggctgttagcccaggaaactcagatacaagctacaacgagaagttcaaagggaaggcaactctgactgtcgataagagcgcgagtacagcttatatggaactttcctcacttcggagcgaagacacagctgtatattattgtaccaggtctcgctacggtaataatgcactggattactggggacaaggaaccctcgttacggtcagttct

SEQ ID NO: 35 - protein, 107 aa, synthetic construct
QIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELK

SEQ ID NO: 36 - protein, 107 aa, synthetic construct
DIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIK

SEQ ID NO: 37 - DNA, 321 bp, synthetic construct
  source 1..321: mol_type = other DNA
cagatcgttcttacgcagagtcctgcttccctttccgcttctgtgggcgaaacagttaccattacatgccgcgcaagcgggaatattcactcttacctggcgtggtatcaacaaaagcaaggtaaatccccacaacttctggtttacaacgccaaaactctggctgatggtgtaccttcacgcttcagcggcagtgggagcgggactcagtactctctgaaaatcaatagccttcaaccagaagattttggctcctattattgccaacacttttggaatactcctccgactttcggggctgggactaagcttgaattgaaa

SEQ ID NO: 38 - DNA, 321 bp, synthetic construct
  source 1..321: mol_type = other DNA
gatatacagttgacgcagagtccatcttctctcagtgctagcgttggagaccgggtgacgattacgtgtagggcgagccaggggattagttccaatatcgtgtggctccaacagaagcctggcaaggctcccaagggccttatttaccacgggactaatctggaatcaggtgtaccaagtcgattctccgggtccggttcaggcacagactataccctcacgataagttcattggagccggaggattttgccacatattattgcgtccagtattcacagttccctccgacatttggtcaggggactaagcttgaaattaag

SEQ ID NO: 39 - protein, 241 aa, synthetic construct
QVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSSGGGGSGGGGSGGGGSQIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELK

SEQ ID NO: 40 - protein, 241 aa, synthetic construct
QIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELKGGGGSGGGGSGGGGSQVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSS

SEQ ID NO: 41 - protein, 239 aa, synthetic construct
DIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIKGGGGSGGGGSGGGGSQLVQSGAEVVKPGASVKVSCKASGYTFTSYWMHWVKQAPGQGLEWIGAVSPGNSDTSYNEKFKGKATLTVDKSASTAYMELSSLRSEDTAVYYCTRSRYGNNALDYWGQGTLVTVSS

SEQ ID NO: 42 - protein, 239 aa, synthetic construct
QLVQSGAEVVKPGASVKVSCKASGYTFTSYWMHWVKQAPGQGLEWIGAVSPGNSDTSYNEKFKGKATLTVDKSASTAYMELSSLRSEDTAVYYCTRSRYGNNALDYWGQGTLVTVSSGGGGSGGGGSGGGGSDIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIK

SEQ ID NO: 43 - DNA, 723 bp, synthetic construct
  source 1..723: mol_type = other DNA
caggtccagttgcaacagtctggccctggtctggtccaacctagtcaatctttgtccattacctgcactgtgtctggtttttctctcacgtcttatggaattcattgggttagacaaagccctgggaaagggttggagtggttgggtgtgatctggcgcggaggtggtactgacagcaacgcagcgtttatgtctcggctttccataaccaaagacaacagcaagtctcaggtgttcttcaagatgaattccctccaggccgacgatacggcgatctattattgtgctcgctccagatacgacgaggaagaaagcatgaactattggggacaggggacctcagtaacagtgtcaagcggtggtggagggagtggagggggtgggtctggtggtggtggtagccagatcgttcttacgcagagtcctgcttccctttccgcttctgtgggcgaaacagttaccattacatgccgcgcaagcgggaatattcactcttacctggcgtggtatcaacaaaagcaaggtaaatccccacaacttctggtttacaacgccaaaactctggctgatggtgtaccttcacgcttcagcggcagtgggagcgggactcagtactctctgaaaatcaatagccttcaaccagaagattttggctcctattattgccaacacttttggaatactcctccgactttcggggctgggactaagcttgaattgaaa

SEQ ID NO: 44 - DNA, 723 bp, synthetic construct
  source 1..723: mol_type = other DNA
caaatcgtgctcactcagagcccagcctctttgtccgcttccgtgggcgaaactgtcaccataacctgccgcgccagtggtaatatacacagctacctggcatggtatcaacaaaagcaaggtaaatctccacaacttcttgtatacaacgccaaaacactggccgatggagttccctctcgtttcagcggcagcggaagtgggactcagtactccctgaaaatcaattccttgcaacctgaagattttggttcctattattgccaacacttttggaataccccgccaacattcggagctggcactaagttggagctgaaaggaggaggcggatcaggcggggggggttccggtggtggtggctctcaggtccaactgcagcagtccggccctggtctggtccaaccttcccaaagtctgtccataacatgcacagtgtcagggttttcattgacgtcttatggaatacattgggttaggcaaagtcctggaaaaggcctcgaatggcttggggtgatctggcgcggaggaggcacagacagcaacgcggcttttatgagtcgtctctcaattactaaagacaactctaagtcacaggtgttcttcaagatgaatagtctccaggcggacgatacagctatctattattgtgctcgctctcgctacgacgaggaagaaagcatgaactattggggacagggcacctctgtaacagtgtcaagc

SEQ ID NO: 45 - DNA, 717 bp, synthetic construct
  source 1..717: mol_type = other DNA
gatatacagttgacgcagagtccatcttctctcagtgctagcgttggagaccgggtgacgattacgtgtagggcgagccaggggattagttccaatatcgtgtggctccaacagaagcctggcaaggctcccaagggccttatttaccacgggactaatctggaatcaggtgtaccaagtcgattctccgggtccggttcaggcacagactataccctcacgataagttcattggagccggaggattttgccacatattattgcgtccagtattcacagttccctccgacatttggtcaggggactaagcttgaaattaagggcggaggtggctccggtggtgggggctcaggcggcgggggctctcaactggttcagtcaggtgctgaagttgtaaaaccaggcgcttctgttaaagtatcctgtaaagctagcggatatacatttaccagttactggatgcactgggtaaagcaggctccaggacaaggactggagtggatcggggctgttagcccaggaaactcagatacaagctacaacgagaagttcaaagggaaggcaactctgactgtcgataagagcgcgagtacagcttatatggaactttcctcacttcggagcgaagacacagctgtatattattgtaccaggtctcgctacggtaataatgcactggattactggggacaaggaaccctcgttacggtcagttct

SEQ ID NO: 46 - DNA, 717 bp, synthetic construct
  source 1..717: mol_type = other DNA
caactggttcagtcaggtgctgaagttgtaaaaccaggcgcttctgttaaagtatcctgtaaagctagcggatatacatttaccagttactggatgcactgggtaaagcaggctccaggacaaggactggagtggatcggggctgttagcccaggaaactcagatacaagctacaacgagaagttcaaagggaaggcaactctgactgtcgataagagcgcgagtacagcttatatggaactttcctcacttcggagcgaagacacagctgtatattattgtaccaggtctcgctacggtaataatgcactggattactggggacaaggaaccctcgttacggtcagttctggcggaggtggctccggtggtgggggctcaggcggcgggggctctgatatacagttgacgcagagtccatcttctctcagtgctagcgttggagaccgggtgacgattacgtgtagggcgagccaggggattagttccaatatcgtgtggctccaacagaagcctggcaaggctcccaagggccttatttaccacgggactaatctggaatcaggtgtaccaagtcgattctccgggtccggttcaggcacagactataccctcacgataagttcattggagccggaggattttgccacatattattgcgtccagtattcacagttccctccgacatttggtcaggggactaagcttgaaattaag

SEQ ID NO: 47 - protein, 489 aa, synthetic construct
MALPVTALLLPLALLLHAARPGSQIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELKGGGGSGGGGSGGGGSQVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 489 aa, synthetic construct
MALPVTALLLPLALLLHAARPGSQVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSSGGGGSGGGGSGGGGSQIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 487 aa, synthetic construct
MALPVTALLLPLALLLHAARPGSDIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIKGGGGSGGGGSGGGGSQLVQSGAEVVKPGASVKVSCKASGYTFTSYWMHWVKQAPGQGLEWIGAVSPGNSDTSYNEKFKGKATLTVDKSASTAYMELSSLRSEDTAVYYCTRSRYGNNALDYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 487 aa, synthetic construct
MALPVTALLLPLALLLHAARPGSQLVQSGAEVVKPGASVKVSCKASGYTFTSYWMHWVKQAPGQGLEWIGAVSPGNSDTSYNEKFKGKATLTVDKSASTAYMELSSLRSEDTAVYYCTRSRYGNNALDYWGQGTLVTVSSGGGGSGGGGSGGGGSDIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 24 aa, synthetic construct
IYIWAPLAGTCGVLLLSLVITLYC

SEQ ID NO: 52 - DNA, 72 bp, synthetic construct
  source 1..72: mol_type = other DNA
atctacatctgggcacccttggctggaacatgcggggtcctgctgctgagcttggtgatcaccctttactgc

SEQ ID NO: 53 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 54 - protein, 45 aa, synthetic construct
TTTPAPRPPTPAPTIASQPLSLRPEACRPAAGGAVHTRGLDFACD

SEQ ID NO: 55 - DNA, 135 bp, synthetic construct
  source 1..135: mol_type = other DNA
accacgacgccagcaccgcgaccaccaacacctgcgcccaccatcgcgtcgcagcccctgtccctgcgcccagaggcgtgcagaccagcagcggggggcgcagtgcacacgagggggctggacttcgcctgtgat

SEQ ID NO: 56 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 339 bp, synthetic construct
  source 1..339: mol_type = other DNA
agagtaaagttcagtaggtccgccgatgccccagcctatcaacaggggcaaaatcaactctacaacgaacttaatctgggacgccgagaggagtacgatgtcttggataagagacgcggcagggaccctgaaatgggcggaaagccaagacggaagaacccccaggaaggtctgtacaatgaacttcagaaagataagatggccgaagcctacagcgagatcggcatgaaaggagagaggcgccgcggcaaagggcatgatggactgtatcagggtctcagtactgctactaaggacacatatgatgccctccacatgcaggccctgccaccaaggtga

SEQ ID NO: 58 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 59 - protein, 42 aa, synthetic construct
KRGRKKLLYIFKQPFMRPVQTTQEEDGCSCRFPEEEEGGCEL

SEQ ID NO: 60 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 61 - protein, 41 aa, synthetic construct
RSKRSRLLHSDYMNMTPRRPGPTRKHYQPYAPPRDFAAYRS

SEQ ID NO: 62 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 63 - protein, 21 aa, synthetic construct
MALPVTALLLPLALLLHAARP

SEQ ID NO: 64 - protein, 116 aa, synthetic construct
QVHLKESGPGLVQPSQTLSLTCTVSGFSLTFYNVHWVRQPTGKGLEWMAIIWNGGKTDYNSGLKSRLSISRDTSKSQVFLKMNSLQSEDTAAYYCVREVGGRFDYWGQGVLVTVSS

SEQ ID NO: 65 - DNA, 348 bp, synthetic construct
  source 1..348: mol_type = other DNA
caagtgcacctcaaagaatccgggcccgggctcgtacaaccgtcccaaacactcagcctgacatgtacagtgtcaggattttccctcacgttttataacgtccattgggtgcgccaaccaactggcaagggccttgaatggatggccatcatctggaacggtggcaaaacggactataacagcggccttaagagcagactctccatatcccgagatactagcaaatcacaggtgtttctcaagatgaattctctccagagcgaggatactgctgcctattattgcgtgcgcgaagtcggcggtcggtttgactattggggacagggcgttctcgtgactgtgagcagt

SEQ ID NO: 66 - protein, 106 aa, synthetic construct
DFQMTQSPASLSASLGETVTIECLASEDIYDILAWYQQKPGKSPQLLIYDASSLHTGVPSRFSGSGSGAQYSLKINSLQPEDFASYYCQSGLSAPWTFGGGTKLEL

SEQ ID NO: 67 - DNA, 318 bp, synthetic construct
  source 1..318: mol_type = other DNA
gattttcaaatgacgcaaagtcctgccagcttgtccgcctcattgggcgagacagttacaatagagtgtctcgccagtgaagatatatatgacatcctggcctggtaccagcaaaagcctggaaagtcaccccaactgcttatttacgacgcttcctcactgcacaccggtgtgccctctcgttttagcggctccggctcaggtgcccaatacagtctgaaaattaactcactccaaccagaggacttcgcatcctactattgccagagcggactcagcgctccatggacctttggcggcgggacgaaattggagctg

SEQ ID NO: 68 - protein, 237 aa, synthetic construct
QVHLKESGPGLVQPSQTLSLTCTVSGFSLTFYNVHWVRQPTGKGLEWMAIIWNGGKTDYNSGLKSRLSISRDTSKSQVFLKMNSLQSEDTAAYYCVREVGGRFDYWGQGVLVTVSSGGGGSGGGGSGGGGSDFQMTQSPASLSASLGETVTIECLASEDIYDILAWYQQKPGKSPQLLIYDASSLHTGVPSRFSGSGSGAQYSLKINSLQPEDFASYYCQSGLSAPWTFGGGTKLEL

SEQ ID NO: 69 - protein, 237 aa, synthetic construct
DFQMTQSPASLSASLGETVTIECLASEDIYDILAWYQQKPGKSPQLLIYDASSLHTGVPSRFSGSGSGAQYSLKINSLQPEDFASYYCQSGLSAPWTFGGGTKLELGGGGSGGGGSGGGGSQVHLKESGPGLVQPSQTLSLTCTVSGFSLTFYNVHWVRQPTGKGLEWMAIIWNGGKTDYNSGLKSRLSISRDTSKSQVFLKMNSLQSEDTAAYYCVREVGGRFDYWGQGVLVTVSS

SEQ ID NO: 70 - DNA, 711 bp, synthetic construct
  source 1..711: mol_type = other DNA
caagtgcacctcaaagaatccgggcccgggctcgtacaaccgtcccaaacactcagcctgacatgtacagtgtcaggattttccctcacgttttataacgtccattgggtgcgccaaccaactggcaagggccttgaatggatggccatcatctggaacggtggcaaaacggactataacagcggccttaagagcagactctccatatcccgagatactagcaaatcacaggtgtttctcaagatgaattctctccagagcgaggatactgctgcctattattgcgtgcgcgaagtcggcggtcggtttgactattggggacagggcgttctcgtgactgtgagcagtggcggggggggctcaggaggaggtggctctggcgggggggggtctgattttcaaatgacgcaaagtcctgccagcttgtccgcctcattgggcgagacagttacaatagagtgtctcgccagtgaagatatatatgacatcctggcctggtaccagcaaaagcctggaaagtcaccccaactgcttatttacgacgcttcctcactgcacaccggtgtgccctctcgttttagcggctccggctcaggtgcccaatacagtctgaaaattaactcactccaaccagaggacttcgcatcctactattgccagagcggactcagcgctccatggacctttggcggcgggacgaaattggagctg

SEQ ID NO: 71 - DNA, 711 bp, synthetic construct
  source 1..711: mol_type = other DNA
gattttcaaatgacccaaagccctgcatcacttagcgccagtctcggcgagaccgtgactatagagtgtctggcctcagaagatatctatgacatccttgcttggtaccagcagaaacccggcaagagcccccaactgctcatatacgacgcatcctctctgcacacgggcgttccttcccgctttagcgggtctggtagcggggcgcaatactccctgaaaattaacagtcttcaacccgaggacttcgcgtcctactattgccagagcggtcttagcgctccatggacatttggtggcggaacgaaacttgagctgggcggtggtggcagtggtggtggtggttcaggcggcggcgggtcacaagttcacctcaaagaaagtggcccaggcctcgtccaaccaagccaaacactctcactgacatgtacggtgagcggattttcactcacattttataacgttcattgggtgcgccaacccacggggaagggacttgaatggatggctattatttggaacggtggtaaaactgactataacagtggacttaagagtaggctcagtatttcacgcgatacaagtaaatctcaggtgtttcttaagatgaattccttgcagtccgaggataccgctgcctattattgcgtgcgagaggtcggtggccgcttcgactattggggtcagggagtgctcgtaacggtgagcagc

SEQ ID NO: 72 - protein, 485 aa, synthetic construct
MALPVTALLLPLALLLHAARPGSQVHLKESGPGLVQPSQTLSLTCTVSGFSLTFYNVHWVRQPTGKGLEWMAIIWNGGKTDYNSGLKSRLSISRDTSKSQVFLKMNSLQSEDTAAYYCVREVGGRFDYWGQGVLVTVSSGGGGSGGGGSGGGGSDFQMTQSPASLSASLGETVTIECLASEDIYDILAWYQQKPGKSPQLLIYDASSLHTGVPSRFSGSGSGAQYSLKINSLQPEDFASYYCQSGLSAPWTFGGGTKLELSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 485 aa, synthetic construct
MALPVTALLLPLALLLHAARPGSDFQMTQSPASLSASLGETVTIECLASEDIYDILAWYQQKPGKSPQLLIYDASSLHTGVPSRFSGSGSGAQYSLKINSLQPEDFASYYCQSGLSAPWTFGGGTKLELGGGGSGGGGSGGGGSQVHLKESGPGLVQPSQTLSLTCTVSGFSLTFYNVHWVRQPTGKGLEWMAIIWNGGKTDYNSGLKSRLSISRDTSKSQVFLKMNSLQSEDTAAYYCVREVGGRFDYWGQGVLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 490 aa, synthetic construct
MALPVTALLLPLALLLHAARPGSEVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSAGGGGSGGGGSGGGGSDIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIERSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 490 aa, synthetic construct
MALPVTALLLPLALLLHAARPGSDIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIERGGGGSGGGGSGGGGSEVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSASGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 119 aa, synthetic construct
EVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSA

SEQ ID NO: 77 - DNA, 357 bp, synthetic construct
  source 1..357: mol_type = other DNA
gaagtgcagctgcagcagagcggcccggaactggaaaaaccgggcgcgagcgtgaaaattagctgcaaagcgagcggctatagctttaccgcgtataacatgaactgggtgaaacagaacaacggcaaaagcctggaatggattggcaacattgatccgtatcatggcggcaccaactataaccagaaatttaaaggcaaagcgaccctgaccgtggataaaagcagcagcaccgcgtatatgcagctgaaaagcctgaccagcgaagattatgcggtgtattattgcgcgcgcggcggcggcgattggagctggtttgcgtattggggccagggcaccctggtgaccgtgagcgcg

SEQ ID NO: 78 - protein, 108 aa, synthetic construct
DIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIER

SEQ ID NO: 79 - DNA, 324 bp, synthetic construct
  source 1..324: mol_type = other DNA
gatattgtgatgacccagagccataaatttatgagcaccagcgtgggcgatcgcgtgagcattacctgcaaagcgagccaggatgtgaacaccgcggtggcgtggtatcagcagaaaccgggccagagcccgaaagtgctgatttattgggcgagcaaccgccataccggcgtgccggatcgctttaccggcagcggcagcggcaccgattataccctgaccattagcagcatgcaggcggaagatctggcgctgtattattgccatcagcattatagcaccccgtggacctttggcggcggcaccaaactggaaattgaacgc

SEQ ID NO: 80 - protein, 242 aa, synthetic construct
EVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSAGGGGSGGGGSGGGGSDIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIER

SEQ ID NO: 81 - protein, 242 aa, synthetic construct
DIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIERGGGGSGGGGSGGGGSEVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSA

SEQ ID NO: 82 - DNA, 726 bp, synthetic construct
  source 1..726: mol_type = other DNA
gaagtgcagctgcagcagagcggcccggaactggaaaaaccgggcgcgagcgtgaaaattagctgcaaagcgagcggctatagctttaccgcgtataacatgaactgggtgaaacagaacaacggcaaaagcctggaatggattggcaacattgatccgtatcatggcggcaccaactataaccagaaatttaaaggcaaagcgaccctgaccgtggataaaagcagcagcaccgcgtatatgcagctgaaaagcctgaccagcgaagattatgcggtgtattattgcgcgcgcggcggcggcgattggagctggtttgcgtattggggccagggcaccctggtgaccgtgagcgcgggcggcggcggcagcggcggcggcggcagcggcggcggcggcagcgatattgtgatgacccagagccataaatttatgagcaccagcgtgggcgatcgcgtgagcattacctgcaaagcgagccaggatgtgaacaccgcggtggcgtggtatcagcagaaaccgggccagagcccgaaagtgctgatttattgggcgagcaaccgccataccggcgtgccggatcgctttaccggcagcggcagcggcaccgattataccctgaccattagcagcatgcaggcggaagatctggcgctgtattattgccatcagcattatagcaccccgtggacctttggcggcggcaccaaactggaaattgaacgc

SEQ ID NO: 83 - DNA, 726 bp, synthetic construct
  source 1..726: mol_type = other DNA
gatattgtgatgacccagagccataaatttatgagcaccagcgtgggcgatcgcgtgagcattacctgcaaagcgagccaggatgtgaacaccgcggtggcgtggtatcagcagaaaccgggccagagcccgaaagtgctgatttattgggcgagcaaccgccataccggcgtgccggatcgctttaccggcagcggcagcggcaccgattataccctgaccattagcagcatgcaggcggaagatctggcgctgtattattgccatcagcattatagcaccccgtggacctttggcggcggcaccaaactggaaattgaacgcggcggcggcggcagcggcggcggcggcagcggcggcggcggcagcgaagtgcagctgcagcagagcggcccggaactggaaaaaccgggcgcgagcgtgaaaattagctgcaaagcgagcggctatagctttaccgcgtataacatgaactgggtgaaacagaacaacggcaaaagcctggaatggattggcaacattgatccgtatcatggcggcaccaactataaccagaaatttaaaggcaaagcgaccctgaccgtggataaaagcagcagcaccgcgtatatgcagctgaaaagcctgaccagcgaagattatgcggtgtattattgcgcgcgcggcggcggcgattggagctggtttgcgtattggggccagggcaccctggtgaccgtgagcgcg

SEQ ID NO: 84 - DNA, 1470 bp, synthetic construct
  source 1..1470: mol_type = other DNA
atggcgctgccggtgaccgcgctgctgctgccgctggcgctgctgctgcatgcggcgcgcccgggcagcgaagtgcagctgcagcagagcggcccggaactggaaaaaccgggcgcgagcgtgaaaattagctgcaaagcgagcggctatagctttaccgcgtataacatgaactgggtgaaacagaacaacggcaaaagcctggaatggattggcaacattgatccgtatcatggcggcaccaactataaccagaaatttaaaggcaaagcgaccctgaccgtggataaaagcagcagcaccgcgtatatgcagctgaaaagcctgaccagcgaagattatgcggtgtattattgcgcgcgcggcggcggcgattggagctggtttgcgtattggggccagggcaccctggtgaccgtgagcgcgggcggcggcggcagcggcggcggcggcagcggcggcggcggcagcgatattgtgatgacccagagccataaatttatgagcaccagcgtgggcgatcgcgtgagcattacctgcaaagcgagccaggatgtgaacaccgcggtggcgtggtatcagcagaaaccgggccagagcccgaaagtgctgatttattgggcgagcaaccgccataccggcgtgccggatcgctttaccggcagcggcagcggcaccgattataccctgaccattagcagcatgcaggcggaagatctggcgctgtattattgccatcagcattatagcaccccgtggacctttggcggcggcaccaaactggaaattgaacgcagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 85 - DNA, 1467 bp, synthetic construct
  source 1..1467: mol_type = other DNA
atggcgctgccggtgaccgcgctgctgctgccgctggcgctgctgctgcatgcggcgcgcccgggcagccaggtgcagctgcagcagagcggcccgggcctggtgcagccgagccagagcctgagcattacctgcaccgtgagcggctttagcctgaccagctatggcattcattgggtgcgccagagcccgggcaaaggcctggaatggctgggcgtgatttggcgcggcggcggcaccgatagcaacgcggcgtttatgagccgcctgagcattaccaaagataacagcaaaagccaggtgttttttaaaatgaacagcctgcaggcggatgataccgcgatttattattgcgcgcgcagccgctatgatgaagaagaaagcatgaactattggggccagggcaccagcgtgaccgtgagcagcggcggcggcggcagcggcggcggcggcagcggcggcggcggcagccagattgtgctgacccagagcccggcgagcctgagcgcgagcgtgggcgaaaccgtgaccattacctgccgcgcgagcggcaacattcatagctatctggcgtggtatcagcagaaacagggcaaaagcccgcagctgctggtgtataacgcgaaaaccctggcggatggcgtgccgagccgctttagcggcagcggcagcggcacccagtatagcctgaaaattaacagcctgcagccggaagattttggcagctattattgccagcatttttggaacaccccgccgacctttggcgcgggcaccaaactggaactgaaaagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 86 - DNA, 1461 bp, synthetic construct
  source 1..1461: mol_type = other DNA
atggcgctgccggtgaccgcgctgctgctgccgctggcgctgctgctgcatgcggcgcgcccgggcagcgatattcagctgacccagagcccgagcagcctgagcgcgagcgtgggcgatcgcgtgaccattacctgccgcgcgagccagggcattagcagcaacattgtgtggctgcagcagaaaccgggcaaagcgccgaaaggcctgatttatcatggcaccaacctggaaagcggcgtgccgagccgctttagcggcagcggcagcggcaccgattataccctgaccattagcagcctggaaccggaagattttgcgacctattattgcgtgcagtatagccagtttccgccgacctttggccagggcaccaaactggaaattaaaggcggcggcggcagcggcggcggcggcagcggcggcggcggcagccagctggtgcagagcggcgcggaagtggtgaaaccgggcgcgagcgtgaaagtgagctgcaaagcgagcggctatacctttaccagctattggatgcattgggtgaaacaggcgccgggccagggcctggaatggattggcgcggtgagcccgggcaacagcgataccagctataacgaaaaatttaaaggcaaagcgaccctgaccgtggataaaagcgcgagcaccgcgtatatggaactgagcagcctgcgcagcgaagataccgcggtgtattattgcacccgcagccgctatggcaacaacgcgctggattattggggccagggcaccctggtgaccgtgagcagcagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 87 - DNA, 1461 bp, synthetic construct
  source 1..1461: mol_type = other DNA
atggcgctgccggtgaccgcgctgctgctgccgctggcgctgctgctgcatgcggcgcgcccgggcagccagctggtgcagagcggcgcggaagtggtgaaaccgggcgcgagcgtgaaagtgagctgcaaagcgagcggctatacctttaccagctattggatgcattgggtgaaacaggcgccgggccagggcctggaatggattggcgcggtgagcccgggcaacagcgataccagctataacgaaaaatttaaaggcaaagcgaccctgaccgtggataaaagcgcgagcaccgcgtatatggaactgagcagcctgcgcagcgaagataccgcggtgtattattgcacccgcagccgctatggcaacaacgcgctggattattggggccagggcaccctggtgaccgtgagcagcggcggcggcggcagcggcggcggcggcagcggcggcggcggcagcgatattcagctgacccagagcccgagcagcctgagcgcgagcgtgggcgatcgcgtgaccattacctgccgcgcgagccagggcattagcagcaacattgtgtggctgcagcagaaaccgggcaaagcgccgaaaggcctgatttatcatggcaccaacctggaaagcggcgtgccgagccgctttagcggcagcggcagcggcaccgattataccctgaccattagcagcctggaaccggaagattttgcgacctattattgcgtgcagtatagccagtttccgccgacctttggccagggcaccaaactggaaattaaaagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 88 - DNA, 1455 bp, synthetic construct
  source 1..1455: mol_type = other DNA
atggcgctgccggtgaccgcgctgctgctgccgctggcgctgctgctgcatgcggcgcgcccgggcagccaggtgcatctgaaagaaagcggcccgggcctggtgcagccgagccagaccctgagcctgacctgcaccgtgagcggctttagcctgaccttttataacgtgcattgggtgcgccagccgaccggcaaaggcctggaatggatggcgattatttggaacggcggcaaaaccgattataacagcggcctgaaaagccgcctgagcattagccgcgataccagcaaaagccaggtgtttctgaaaatgaacagcctgcagagcgaagataccgcggcgtattattgcgtgcgcgaagtgggcggccgctttgattattggggccagggcgtgctggtgaccgtgagcagcggcggcggcggcagcggcggcggcggcagcggcggcggcggcagcgattttcagatgacccagagcccggcgagcctgagcgcgagcctgggcgaaaccgtgaccattgaatgcctggcgagcgaagatatttatgatattctggcgtggtatcagcagaaaccgggcaaaagcccgcagctgctgatttatgatgcgagcagcctgcataccggcgtgccgagccgctttagcggcagcggcagcggcgcgcagtatagcctgaaaattaacagcctgcagccggaagattttgcgagctattattgccagagcggcctgagcgcgccgtggacctttggcggcggcaccaaactggaactgagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 89 - DNA, 1455 bp, synthetic construct
  source 1..1455: mol_type = other DNA
atggcgctgccggtgaccgcgctgctgctgccgctggcgctgctgctgcatgcggcgcgcccgggcagcgattttcagatgacccagagcccggcgagcctgagcgcgagcctgggcgaaaccgtgaccattgaatgcctggcgagcgaagatatttatgatattctggcgtggtatcagcagaaaccgggcaaaagcccgcagctgctgatttatgatgcgagcagcctgcataccggcgtgccgagccgctttagcggcagcggcagcggcgcgcagtatagcctgaaaattaacagcctgcagccggaagattttgcgagctattattgccagagcggcctgagcgcgccgtggacctttggcggcggcaccaaactggaactgggcggcggcggcagcggcggcggcggcagcggcggcggcggcagccaggtgcatctgaaagaaagcggcccgggcctggtgcagccgagccagaccctgagcctgacctgcaccgtgagcggctttagcctgaccttttataacgtgcattgggtgcgccagccgaccggcaaaggcctggaatggatggcgattatttggaacggcggcaaaaccgattataacagcggcctgaaaagccgcctgagcattagccgcgataccagcaaaagccaggtgtttctgaaaatgaacagcctgcagagcgaagataccgcggcgtattattgcgtgcgcgaagtgggcggccgctttgattattggggccagggcgtgctggtgaccgtgagcagcagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 90 - DNA, 1470 bp, synthetic construct; identical to SEQ ID NO: 84
  source 1..1470: mol_type = other DNA

SEQ ID NO: 91 - DNA, 1470 bp, synthetic construct
  source 1..1470: mol_type = other DNA
atggcgctgccggtgaccgcgctgctgctgccgctggcgctgctgctgcatgcggcgcgcccgggcagcgatattgtgatgacccagagccataaatttatgagcaccagcgtgggcgatcgcgtgagcattacctgcaaagcgagccaggatgtgaacaccgcggtggcgtggtatcagcagaaaccgggccagagcccgaaagtgctgatttattgggcgagcaaccgccataccggcgtgccggatcgctttaccggcagcggcagcggcaccgattataccctgaccattagcagcatgcaggcggaagatctggcgctgtattattgccatcagcattatagcaccccgtggacctttggcggcggcaccaaactggaaattgaacgcggcggcggcggcagcggcggcggcggcagcggcggcggcggcagcgaagtgcagctgcagcagagcggcccggaactggaaaaaccgggcgcgagcgtgaaaattagctgcaaagcgagcggctatagctttaccgcgtataacatgaactgggtgaaacagaacaacggcaaaagcctggaatggattggcaacattgatccgtatcatggcggcaccaactataaccagaaatttaaaggcaaagcgaccctgaccgtggataaaagcagcagcaccgcgtatatgcagctgaaaagcctgaccagcgaagattatgcggtgtattattgcgcgcgcggcggcggcgattggagctggtttgcgtattggggccagggcaccctggtgaccgtgagcgcgagcggcaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtataaacagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

(19) United States
(12) Patent Application Publication
(10) Pub. No.: US 2024/0376197 A1
(43) Pub. Date: Nov. 14, 2024

(54) COMPOSITIONS AND METHODS FOR CHIMERIC ANTIGEN RECEPTORS SPECIFIC TO B CELL RECEPTORS

(71) Applicants: The Trustees of the University of Pennsylvania, Philadelphia, PA (US); Institute of Applied Bioscience (INAB) / Centre For Research and Technology Hellas (CERTH), Thessaloniki (GR); Ospedale San Raffaele S.R.L. (OSR), Milan (IT); Emory University, Atlanta, GA (US)

(72) Inventors: Marco Ruella, Ardmore, PA (US); Stephen Schuster, Springfield, PA (US); Kostas Stamatopoulos, Thessaloniki (GR); Paolo Prospero Ghia, Milano (IT); Ignacio Sanz, Atlanta, GA (US); Ivan Cohen, Philadelphia, PA (US)

(21) Appl. No.: 18/684,598

(22) PCT Filed: Aug. 18, 2022
(86) PCT No.: PCT/US2022/075140

§ 371 Date: Feb. 16, 2024

Related U.S. Application Data
(60) Provisional application No. 63/234,514, filed on Aug. 18, 2021.

(51) Int. Cl. C07K 16/28; A61K 39/00; A61P 35/00; A61P 35/02; C07K 14/705; C07K 14/725

(52) U.S. Cl. CPC C07K 16/2803; A61K 39/4611; A61K 39/4631; A61K 39/464412; A61P 35/00; A61P 35/02; C07K 14/7051; C07K 14/70517; C07K 14/70578; A61K 2239/17; A61K 2239/21; A61K 2239/22; C07K 2317/622; C07K 2319/02; C07K 2319/03

(57) ABSTRACT

The present invention relates to compositions and methods for treating or preventing a hematologic cancer or autoimmune disease of a mammal using anti-BCR CARs. One aspect includes a modified T cell and pharmaceutical compositions comprising the modified cells for adoptive cell therapy and treating a cancer or autoimmune disease associated with B cells comprising enriched stereotyped BCRs.

### CROSS-REFERENCE TO RELATED APPLICATIONS

This application claims priority to U.S. Provisional Application No. 63/234,514 filed Aug. 18, 2021, which is herein incorporated by reference in its entirety.

### REFERENCE TO AN ELECTRONIC SEQUENCE LISTING

The contents of the electronic sequence listing (046483-7330WO1-02992 Sequence Listing.xml; Size: 108,648 bytes; and Date of Creation: Aug. 17, 2022) is herein incorporated by reference in its entirety.

### BACKGROUND OF THE INVENTION

Malignant or pathogenic B cell clones in lymphomas, leukemias, myelomas or other hematological cancers can be identified by the expression of specific, recurrent or enriched stereotyped B-cell receptors (BCRs) that can be involved in the pathogenesis of the B-cell malignancy. Furthermore, several autoimmune disorders are characterized by the enrichment of specific B cell receptors that are thought to be linked to the pathogenesis of the autoimmune disease. Existing therapies targeting B cells associated with cancers or autoimmune diseases have drawbacks since they are not able to specifically target the pathogenic clone, but rather deplete the entire B-cell compartment. For example, CD19-directed chimeric antigen receptors (CARs) target all patient B cells and often result in B-cell aplasia with patients needing long-term IgG infusions to prevent recurrent infections. Moreover, without B-cells, patients poorly respond to vaccines.

There remains an unmet need for effective therapies that target pathogenic B cell clones associated with hematologic cancers or autoimmune diseases and spare normal B-cells or plasma cells.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

The foregoing and other features and advantages of the present invention will be more fully understood from the following detailed description of illustrative embodiments taken in conjunction with the accompanying drawings.

FIG. 1 illustrates the percentage of patients with various hematologic malignancies that carry the immunoglobulin heavy regions (IGHV) 4-34-containing B Cell Receptor. Among patients with primary vitreoretinal lymphoma (PVRL), more than 60% of these patients carry this specific BCR, while in primary central nervous system lymphoma (PCNSL), Activated B cell-like diffuse large B cell lymphoma (ABC-DLBCL) and Hairy cell leukemia-variant (HCLv), approximately 30-40% of patients carry this specific BCR in their malignant tumor cells. In contrast, IGHV4-34-containing BCRs are found in a small minority of normally healthy B cells (<5%) (Belhouaci et al, Blood Advances, 2020). PVRL: primary vitreoretinal lymphoma; PCNSL: primary central nervous system lymphoma; DLBCL-ABC: diffuse large B cell lymphoma-activated B cell like; DLBCL-GCB: diffuse large B cell lymphoma-germinal center like; BL: Burkitt's lymphoma; CLL: chronic lyumphocytic leukemia; MCL: mantle cell lymphoma; SMZL: splenic marginal zone lymphoma; OAMZL: ocular adnexal marginal zone lymphomas; CBL-MZ: clonal B cell lymphocytosis of marginal zone origin; HCLc: hairy cell leukemia-classical; HCLv: hairy cell leukemia-variant.

FIG. 2A illustrates the enrichment of specific IGHV in a large cohort of patients with chronic lymphocytic leukemia (CLL). The red bars denote the enrichment of the following IGHV regions in 5-12% of CLL patients each: IGHV1-69, IGHV3-23, IGHV4-34.

FIG. 2B Illustrates that approximately 17-18% of patients from a different cohort of CLL carry the IGLV3-21 BCR containing a mutation in the R110 site (Maity et al, PNAS 2020; Muggen et al, Immunity & Ageing 2019).

FIG. 3 is a schematic illustrating development of CAR to target enriched stereotyped BCRs. Chimeric antigen receptor T cells against enriched stereotyped BCR will specifically kill the tumor clone while sparing normal B cells; therefore, there will be no B-cell aplasia, no need for intravenous (IV) immunoglobulins for life and reduced opportunistic infections. Moreover, signaling downstream of the BCR is known to drive tumor cell survival and proliferation. Thus, unlike targeting CD19, which is not required for tumor B cell survival and proliferation, targeting the enriched stereotyped BCR will also mean targeting an “Achilles' Heel” of leukemia/lymphoma cells (L/L) i.e., the BCR that they need to survive. Therefore, this strategy could potentially reduce antigen-negative escape (e.g. CD19-neg relapses). IV: intravenous; L/L: leukemia/lymphoma cells; StBCR: stereotypic/enriched BCR.

FIG. 4 is a schematic illustrating the development of CAR T cells to specifically target the tumor B cell clones. This strategy disclosed herein has several advantages compared to CD19-directed CAR T cell therapy, including the following: (i) while CD19-directed CAR T cell therapy will lead to the killing of all mature B cells (B cell aplasia); targeting only B cells carrying the disease-specific ‘stereotyped’ or ‘enriched’ BCR will lead to the elimination of the tumor B cell clone while sparing all other poly-clonal B cells carrying diverse BCRs. These poly-clonal B cells can then thrive in patients after the anti-BCR CAR T cell infusion and protect the patient from life-threatening opportunistic infections; and (ii) while CD19-directed CAR T cell therapy will initially kill tumor B cells carrying CD19, it is common that CD19-negative tumor cells develop even in the presence of CD19-directed CAR T cells, since CD19 is not a molecule that is required for tumor B cell survival or proliferation. In contrast, given the critical role of the BCR in promoting B cell signaling, survival and proliferation, BCR-negative tumor B cells will be less common. BCR: B cell receptor.

FIG. 5 is a schematic showing exemplary embodiments of the CAR constructs. The CARs disclosed herein comprise single-chain variable fragments (scFv) derived from antibodies that recognize specific immunoglobulin heavy regions (IGHV). This portion of the CAR serves to direct the CAR T cells to pathogenic B cells that express specific BCRs. Each construct was cloned with antigen binding domain (the scFv) in both the Light-to-Heavy (L2H) and Heavy-to-Light (H2L) orientations. The various scFvs that recognize specific BCRs were paired with the 41BB-CD3z intracellular signaling domains which have been used previously in an anti-CD19 CAR. scFv: single-chain variable fragment. 41BB: intracellular domain of 41BB.

FIG. 6 is a schematic showing the approach disclosed herein to develop tumor B cell lines that express the stereotyped BCR. As a first step, the endogenous immunoglobulin heavy (IGH) and light (IGL) chains were knocked-out using CRISPR/Cas9, followed by lentiviral transduction with immunoglobulin heavy and light chains encoding stereotyped or enriched BCRs (IGLV3-21 R110; IGHV1-69; IGHV4-34; IGHV3-23). The sequences encoded by these BCRs were derived from patients with hematologic malignancies. CRISPR: clustered regularly interspaced short palindromic repeats. LV: lentiviral; IgM C: IgM constant region; IgG1 C: IgG1 constant region.

FIGS. 7A-7B relate to anti-BCR CART manufacturing for CAR T cells carrying a variety of CARs that can recognize stereotyped or enriched BCRs. FIG. 7A shows (i) the total T cell numbers following stimulation with CD3/CD28-coated magnetic beads; the cell volume of the T cells following stimulation, showing an initial enlargement of cell size following by a rest-down period; and the population doublings of the T cells following stimulation. FIG. 7B shows the successful expression of the anti-BCR CARs 8 days following stimulation. AVA=anti-3-21 IGLV; hG6.3=anti-1-69 IGHV; 9G4=anti-IGHV4-34; 3C9=anti-3-23 IGLV.

FIGS. 8A-8B show the in-vitro assays of anti-BCR CAR T cell efficacy. FIG. 8A shows that culturing tumor B cells that carry either WT BCR, stereotyped BCRs, or which have CD19 depletion, with CAR T cells that are specific for either CD19, VL3-21-R110, VH1-69 or VH4-34, leads to the specific decrease in live tumor cell numbers in the expected conditions: CART19 T cells lead to the depletion of all tumor B cells except those that are CD19-KO; CART3-21 leads to the depletion of Jekol VL3-21* but not other tumor B cells; CART1-69 leads to the depletion of Jekol VH1-69 but not other tumor B cells; and CART4-34 leads to the depletion of Jekol VH4-34 but not other tumor B cells. These data clearly demonstrate the specificity of the Anti-BCR CAR T cells of the present invention. FIG. 8B shows the proliferation of the various CAR T cells after 6 days of culture with various tumor B cells. These results demonstrate that CAR T cells specifically respond and proliferate when they encounter an antigen (CD19 or stereotyped/enriched BCR) that they can recognize through the CAR which they express.

FIG. 9 shows the specific anti-tumor effect of the CAR T cells of the invention against patient-derived tumor cells. In this experiment, B cells from a healthy patient, or tumor B cells from a patient with CLL that express IGHV1-69+ BCR, or tumor B cells from a patient with CLL that express IGHV4-34+ BCR, were co-cultured with CART19, or CART1-69 or CART4-34. The data show that, as expected, CART19 exhibits strong cytotoxicity against all types of B cells, including healthy B cells and tumor B cells from both patients. In contrast, CART1-69 shows specific cytotoxicity against the IGHV1-69+ CLL tumor B cells, while sparing normal healthy B cells. Similarly, CART4-34 shows specific cytotoxicity against the IGHV4-34+ CLL tumor B cells, while sparing healthy B cells. ND580 B Cells: normal/healthy donor B cells.

FIG. 10 shows the in-vitro cytotoxicity of CART3-23 CAR T cells against a diffuse large B cells lymphoma (DLBCL) cell line that expresses IGHV3-23+ BCR (OCI-Ly18), but does not show appreciable cytotoxicity against HBL1, another DLBCL cell line that expresses IGHV4-34+ BCR. These results show the specific nature of CART3-23 to specifically target B cells that carry an IGHV3-23+ BCR.

FIG. 11 shows the ability of CART4-34 to control tumors in-vivo. A cell line (Mec1, CLL) was engineered with deletion of its endogenous Immunoglobulin heavy and light chain, followed by overexpression of a BCR containing the IGHV4-34 region. This IGHV4-34+ cell line was injected intravenously into mice on Day 0, followed by intravenous injection of CART19 or CART4-34 T cells on Day 13 of the experiment (Untransduced (UTD) T cells served as negative control). This figure shows that both CART19 and CART4-34 can curtail tumor growth to similar levels.

FIG. 12 shows a heatmap of IGHV gene frequency and BCR subtypes in autoimmune diseases. Systemic lupus erythematosus is enriched in IGHV4-34 BCRs; Behcet's disease instead is enriched in IGHV 1-69; thrombotic thrombocytopenia purpura is enriched in IGHV 1-69. TTP: thrombotic thrombocytopenia purpura; SLE: systemic lupus erythematosus; CD: Crohn's disease; BD: Behcet's disease; EGPA: eosinophilic granulomatosis with polyangiitis; TTP: thrombotic thrombocytopenia purpura; AAV: ANCA-associated vasculitis; IgAV: IgA vasculitis; IgAV: IgA vasculitis. Figure adapted from Bashford-Rogers, R. J. M. et al., Nature, 574 (7776): 122-1261-29 (2019).

FIG. 13 is a map showing epitope specificities of anti-ADAMTS13 scFv based on information from patient's with thrombotic thrombocytopenia purpura (TTP). Figure reproduced from Ostertag, E. M. et al., Transfusion 56, 1763-1774 (2016).

FIG. 14 illustrates some of the characteristics of stereotyped BCR in chronic lymphocytic leukemia (CLL) patients: shared B cell receptor sequences among chronic lymphocytic leukemia patients; 41% of CLL patients have malignant B cells with stereotyped BCRs; and some subset of CLL patients with stereotyped BCRs show poor prognosis. CLL subset 2: IGHV3-21 and IGLV3-21; Unique HCDR3 sequences; Unfavorable prognosis with BCR with IGLV3-21. Reproduced from Maity, P. C. et al., Proc. Natl. Acad. Sci. U.S.A., 117:4320-4327 (2020), Stamatopoulos, B. et al., Clin Cancer Res, 24:5048-5057 (2018) and Agathangelidis, A. et al. Blood 119, 4467-4475 (2012).

FIG. 15 shows sequence logos for stereotyped BCRs in subsets 2, 4, and 6 corresponding to VL 3-21, VH 4-34, and VH 1-69, respectively, are enriched in chronic lymphocytic leukemia (CLL) and mantle cell lymphoma (MCL). Reproduced from Agathangelidis, A. et al. Blood 119, 4467-4475 (2012).

FIG. 16 is a schematic illustrating development of CAR to target stereotyped BCRs. Chimeric antigen receptor T cells against stereotyped BCR specifically kill the tumor clone while sparing normal B cells; therefore, there will be no B-cell aplasia, no need for IV immunoglobulins for life and reduced opportunistic infections. Moreover, targeting the stereotyped BCR will also mean targeting an “Achilles' Heel” of CLL cells i.e., the BCR that they need to survive (differently than CD19); therefore, it is contemplated herein that this strategy will reduce antigen-negative escape (e.g. CD19-neg relapses). The “R110 BCR” is the IGLV3-21 with R110 mutation.

FIG. 17 is a schematic representation (example) of the vector map for pTRPE AVA L2H CAR and corresponding sequence of the CAR (SEQ ID NO: 47) in accordance with one embodiment of a CAR.

FIG. 18 are graphs showing in vitro killing experiment: specific killing of VL 3-21. Legend: UTD-untransduced T cells; CART19-anti-CD19 CAR T cells; 3.21*CAR-anti-IGLV3-21 R110 BCR CAR T cells; and 1.69 CAR-anti-IGHV1-69 BCR CAR T cells.

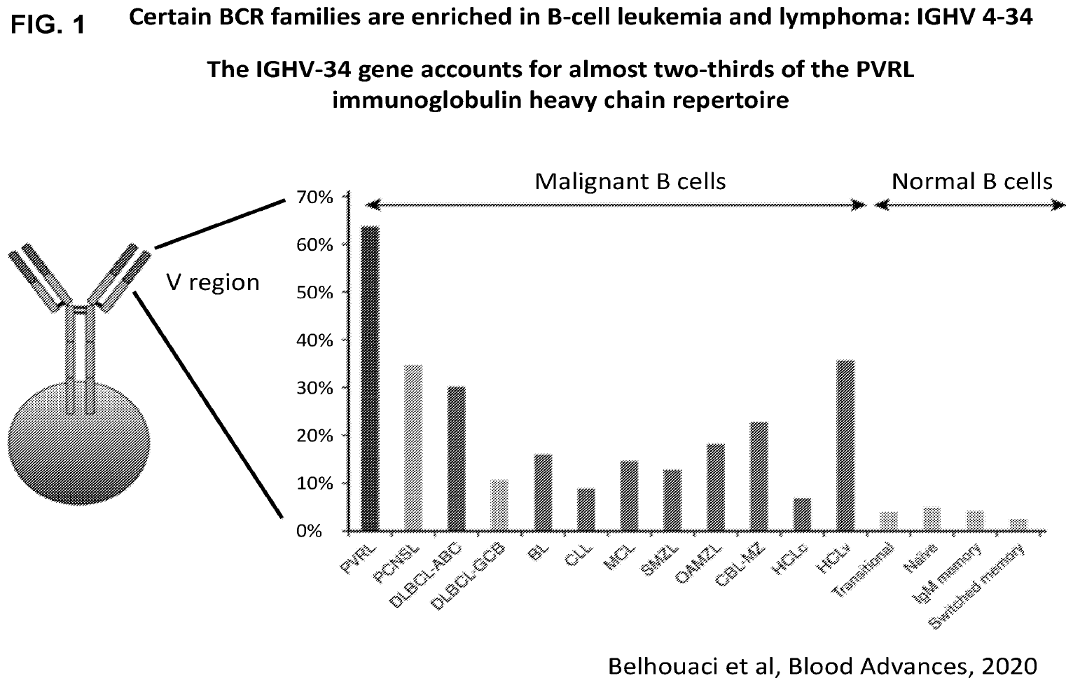

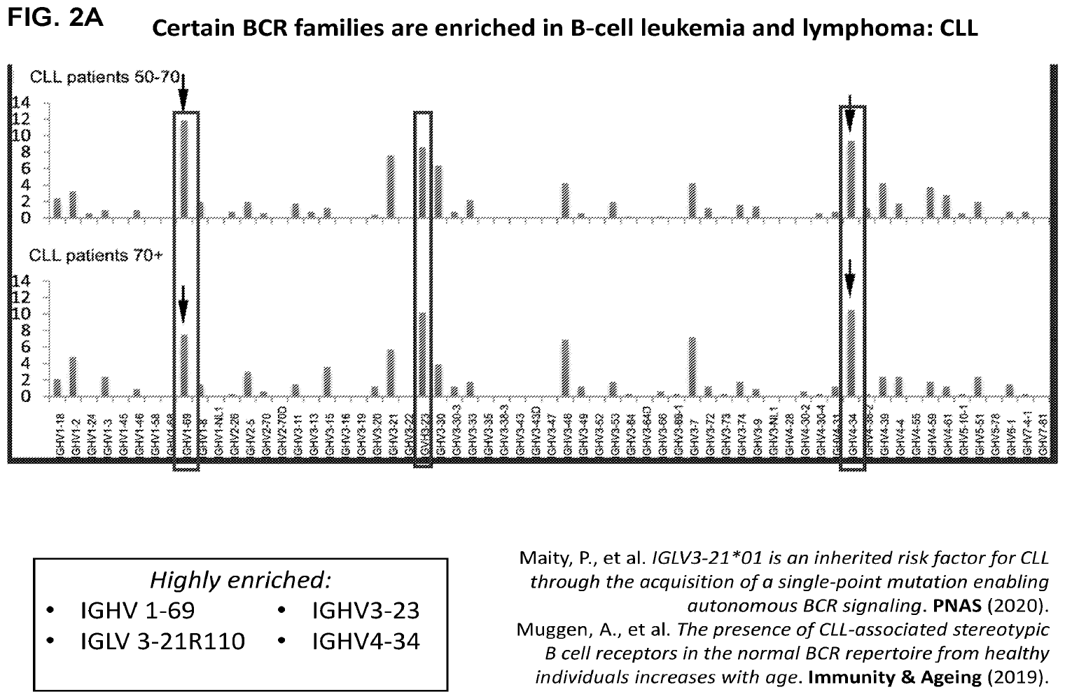

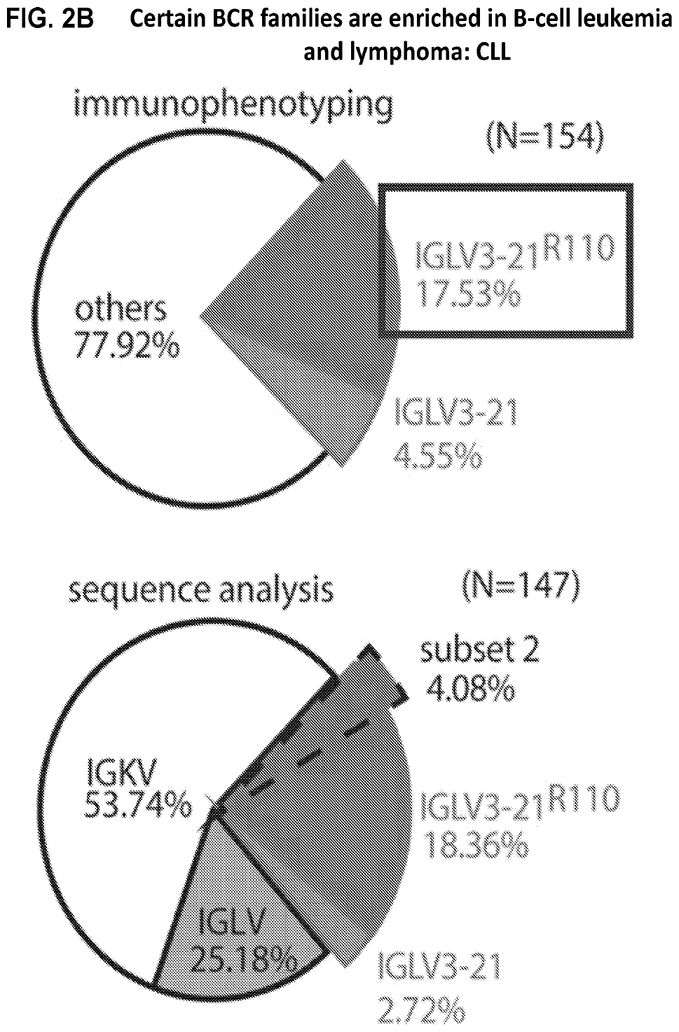

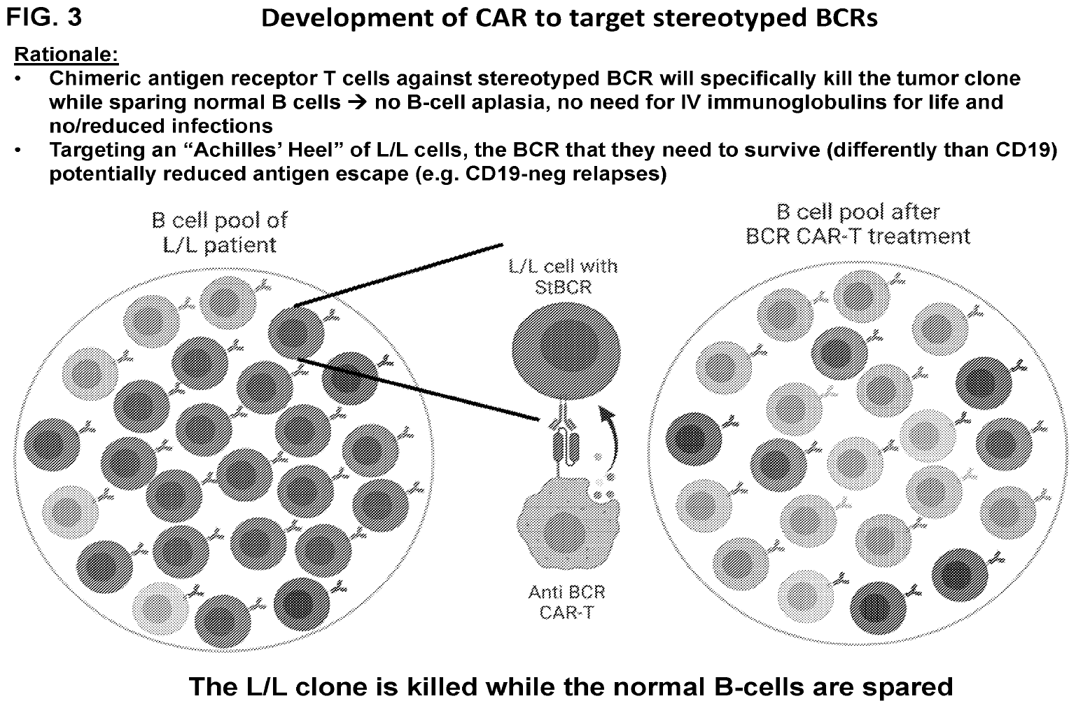

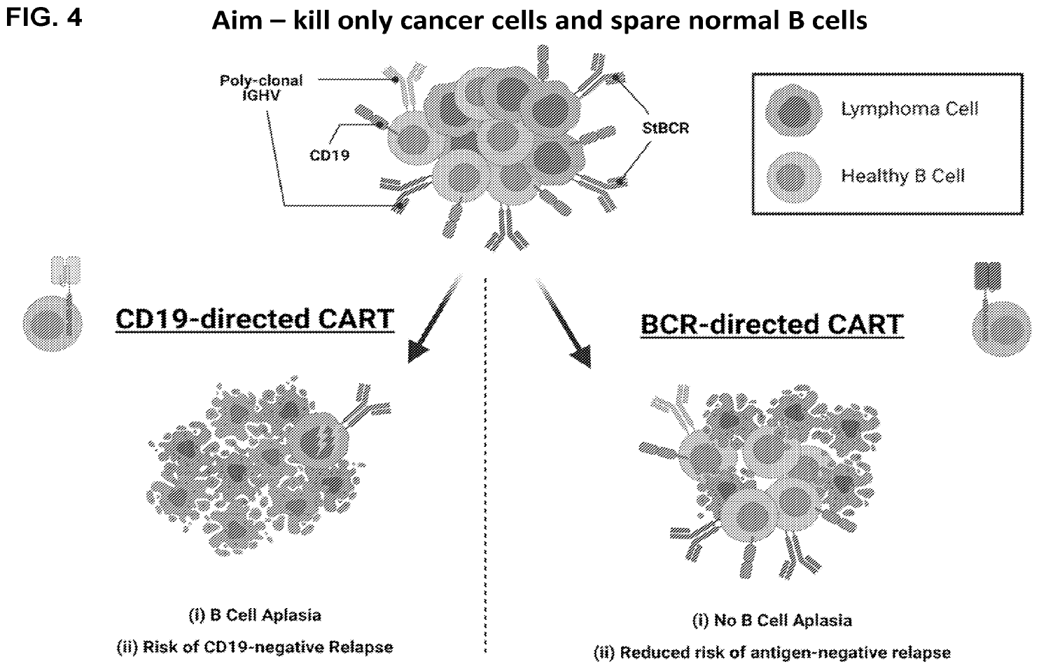

### DETAILED DESCRIPTION

The present invention relates to a strategy of adoptive cell transfer of cells (e.g., immune cells such as, for example, T cells) transduced to express a chimeric antigen receptor (CAR). CARs are molecules that combine antigen-binding domain-based specificity for a target antigen with a T cell receptor-activating intracellular domain to generate a chimeric protein that exhibits a specific anti-target cellular immune activity. In some aspects, the present invention includes a type of cellular therapy where T cells are genetically modified to express a CAR against a stereotyped B cell Receptor (e.g., CAR against VH4-34, VL3-21, VH3-23 or VH1-69 BCRs) and the CAR T cell can be infused to a recipient in need thereof. The infused cells are able to target enriched/stereotyped B cells in the recipient, thereby targeting e.g., malignant or pathogenic B-cell clones in e.g., lymphoma, leukemia, myeloma, other hematologic malignancies or autoimmune disease while preserving normal B-cells of the recipient.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook, and Harlow et al., Antibodies: A Laboratory Manual, Chapter 14, Cold Spring Harbor Laboratory, Cold Spring Harbor (2013, 2nd edition).

Methods and techniques using T Cells with chimeric antigen receptors (CAR T cells) are described in e.g., Ruella, M. et al., Dual CD19 and CD123 targeting prevents antigen-loss relapses after CD19-directed immunotherapies. J. Clin. Invest., 126 (10): 3814-3826 (2016) and Kalos, M. et al., T Cells with Chimeric Antigen Receptors Have Potent Antitumor Effects and Can Establish Memory in Patients with Advanced Leukemia, Science Translational Medicine, 3 (95), 95ra73: 1-11 (2011), the contents of which are hereby incorporated by reference in their entireties.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

So that the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “adaptor molecule” refers to a polypeptide with a sequence that permits interaction with two or more molecules, and in certain embodiments, promotes activation or inactivation of a cytotoxic cell.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λlight chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “anti-cancer effect” as used herein, refers to a biological effect which can be manifested by a decrease in the number of tumor cells, a decrease in the number of metastases, an increase in life expectancy, and/or amelioration of various physiological symptoms associated with the cancerous condition (e.g., hematologic cancer). An “anti-cancer effect” can also be manifested by the ability of the peptides, polynucleotides, cells and/or CARs of the invention in prevention of the occurrence of cancer in the first place.

The term “autoimmune disease” as used herein is defined as a disorder that results from an autoimmune response. An autoimmune disease is the result of an inappropriate or excessive response to a self-antigen. Examples of autoimmune diseases include but are not limited to, systemic lupus erythematosus (SLE), Crohn's disease (CD), Behcet's disease (BD), eosinophilic granulomatosis with polyangiitis (EGPA), thrombotic thrombocytopenia purpura (TTP), ANCA-associated vasculitis (AAV), IgA vasculitis (IgAV), IgA vasculitis (IgAV), Addison's disease, alopecia areata, ankylosing spondylitis, autoimmune hepatitis, autoimmune parotitis, diabetes (Type I), epididymitis, glomerulonephritis, Graves' disease, Guillain-Barre syndrome, Hashimoto's disease, hemolytic anemia, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different organism of the same species.

“Xenogeneic” refers to a graft derived from an organism of a different species.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. CARs may also comprise an intracellular activation domain, a transmembrane domain and an extracellular domain comprising a tumor associated antigen binding region. In some aspects, CARs comprise fusions of single-chain variable fragments (scFv) derived from monoclonal antibodies, fused to transmembrane and intracellular domain.

The term “chimeric intracellular signaling molecule” refers to recombinant receptor comprising one or more intracellular domains of one or more co-stimulatory molecules. The chimeric intracellular signaling molecule substantially lacks an extracellular domain. In some embodiments, the chimeric intracellular signaling molecule comprises additional domains, such as a transmembrane domain, a detectable tag, and a spacer domain.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, for example, one or more amino acid residues within the complementary-determining regions (CDRs) of an antibody or antigen binding fragment thereof can be replaced with other amino acid residues from the same side chain family and the altered antibody can be tested for the ability to bind antigens using the functional assays described herein.

The term “cytotoxic” or “cytotoxicity” refers to killing or damaging cells. In one embodiment, cytotoxicity of the modified cells is improved, e.g. increased cytolytic activity of T cells.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-cancer activity as determined by any method suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. As applied to the nucleic acid or protein, “homologous” as used herein refers to a sequence that has about 50% sequence identity. More preferably, the homologous sequence has about 75% sequence identity, even more preferably, has at least about 80%, 85%, 90%, 91%, 92%, 93%, 94%, 95%, 96%, 97%, 98%, 99% sequence identity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) (e.g., complementary-determining region 1 (CDR1) and/or complementary-determining region 2 (CDR2) and/or complementary-determining region 3 (CDR3)) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody or antigen binding fragment thereof, where the whole molecule is of human origin or consists of an amino acid sequence identical to a human form of the antibody or antigen binding fragment thereof.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of a composition includes, e.g., subcutaneous (s.c.), intravenous (i.v.), intramuscular (i.m.), intrasternal injection, or infusion techniques.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein with respect to an antibody or antigen binding fragment thereof, is meant an antibody or antigen binding fragment thereof which recognizes a specific antigen, but does not substantially recognize or bind other molecules in a sample. For example, an antibody or antigen binding fragment thereof that specifically binds to an antigen from one species may also bind to that antigen from one or more species. But, such cross-species reactivity does not itself alter the classification of an antibody or antigen binding fragment thereof as specific. In another example, an antibody or antigen binding fragment thereof that specifically binds to an antigen may also bind to different allelic forms of the antigen. However, such cross reactivity does not itself alter the classification of an antibody or antigen binding fragment thereof as specific. In some instances, the terms “specific binding” or “specifically binding,” can be used in reference to the interaction of an antibody or antigen binding fragment thereof, a protein, or a peptide with a second chemical species, to mean that the interaction is dependent upon the presence of a particular structure (e.g., an antigenic determinant or epitope) on the chemical species; for example, an antibody or antigen binding fragment thereof recognizes and binds to a specific protein structure rather than to proteins generally. If an antibody is specific for epitope “A”, the presence of a molecule containing epitope A (or free, unlabeled A), in a reaction containing labeled “A” and the antibody or antigen binding fragment thereof, will reduce the amount of labeled A bound to the antibody or antigen binding fragment thereof.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder (e.g., cancer; autoimmune disease) experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention includes compositions and methods for the treatment or prevention of B cell cancers (including but not limited to lymphoma, myeloma, leukemia) and/or B cell-mediated autoimmune diseases. According to the invention, immune cells including, but not limited to, T cells (including, but not limited to, natural killer T (NKT) cells and gamma-delta T cells), natural killer (NK) cells, and macrophages, are modified for adoptive cell (e.g., T cell) therapy by expressing CAR comprising an antigen binding domain specifically recognizing and binding disease-specific or stereotyped B cell receptors expressed by cells (e.g., cancerous B cells of chronic lymphocytic leukemia (CLL) patients). The modified cells (e.g., modified T cells) of this invention are specific for disease-associated or stereotyped B cell receptors and have improved cytotoxicity and efficacy against B cells having stereotyped B cell receptors.

For example, many B-cell non-Hodgkin lymphomas express restricted or ‘stereotyped’ immunoglobulin variable heavy and light chain genes within their B cell receptors (BCRs). One third of CLL patients express stereotyped receptors, and stereotyped receptors are found in significant fractions of other B-cell non-Hodgkin lymphoma patients, including 33.9% of diffuse large B-cell lymphoma (DLBCL) patients, 46.3% of mantle cell lymphoma (MCL) patients and 45.8% of splenic marginal zone lymphoma (SMZL) patients. While not wishing to be bound by theory, these findings suggest that antigen(s) may play a role in the selection and expansion of neoplastic clones. For example, VL 3.21 (including VL 3.21 with the R110 mutation), VH 1.69, VH 4.34, VH3.23, and others are highly enriched in these diseases. These conserved antigens present novel and tumor-selective antigens for CAR therapy. The CARs of the present invention targeting such antigens represent a more tumor-selective cell therapy than e.g., CD19-directed CARs, which target all mature B-cells and result in patients needing lifelong IgG infusions to prevent infection. In some embodiment, these BCR isoform CARs of the present invention would target a fraction of the B-cell population and therefore have reduced off-tumor toxicity.

In other embodiments, the CAR T cells of the present invention also can be used to specifically ablate autoimmune B cell clones. While not wishing to be bound by theory, several autoimmune disorders are characterized by the enrichment of specific BCRs that are thought to be linked to the pathogenesis of the disease. In particular, the VH 1.69 antibodies are enriched in thrombotic thrombocytopenia purpura (TTP) and Bechet Disease, VH 4.34 in systemic lupus erythematosus (SLE), eosinophilic granulomatosis with polyangiitis (EGPA) and Crohn's disease (CD). Thus, in some embodiment, the CAR T cells of the invention can specifically deplete the B-cell and plasma cells expressing and producing the antibodies that contribute to the pathogenesis of such autoimmune diseases.

Reported data from each of CD19, CD20, and CD22 CAR T-cell therapies in B-cell malignancies suggests that resistance to this class of therapeutics can be due to antigen escape i.e., the emergence of tumors with loss or downregulation of the target antigen.

Accordingly, in still further embodiments, the CAR cells (e.g., CAR T cells) of the present invention can be used, alone or in combination with other treatments, to reduce or eliminate antigen escape by B cell cancers (e.g., cancerous B cells of CLL patients) by targeting an essential protein for B-cell leukemia and lymphoma survival, i.e. the BCR.

### Chimeric Antigen Receptor (CAR)

In one aspect, the present invention provides a chimeric antigen receptor (CAR) comprising an antigen binding domain, a transmembrane domain, and an intracellular signaling domain, wherein the antigen binding domain specifically binds to an enriched stereotyped B cell receptor (BCR).

### Antigen Binding Domain

The antigen binding domain of a CAR is an extracellular region of the CAR for binding to a specific target antigen including proteins, carbohydrates, and glycolipids. The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof.

In some embodiments, the antigen binding domain is selected from the group consisting of an antibody, an antigen binding fragment (Fab), and a single-chain variable fragment (scFv).

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain comprises an scFv having the configuration from N-terminus to C-terminus, VH-linker-VL. In some embodiments, the antigen binding domain comprises an scFv having the configuration from N-terminus to C-terminus, VL-linker-VH. Those of ordinary skill in the art would be able to select the appropriate configuration for use in the present invention.

In some embodiments, the linker is rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 1), (GGGS)ₙ (SEQ ID NO: 2), and (GGGGS)ₙ (SEQ ID NO: 3), where n represents an integer of at least 1. Exemplary linker sequences can comprise amino acid sequences including, without limitation, GGSG (SEQ ID NO: 4), GGSGG (SEQ ID NO: 5), GSGSG (SEQ ID NO: 6), GSGGG (SEQ ID NO: 7), GGGSG (SEQ ID NO: 8), GSSSG (SEQ ID NO: 9), GGGGS (SEQ ID NO: 10), GGGGSGGGGSGGGGS (SEQ ID NO: 11) and the like. Those of ordinary skill in the art would be able to select the appropriate linker sequence for use in the present invention. In one embodiment, an antigen binding domain of the present invention comprises a heavy chain variable region (VH) and a light chain variable region (VL), wherein the VH and VL is separated by the linker sequence having the amino acid sequence of SEQ ID NO: 11, which may be encoded by the nucleic acid sequence GGTGGCGGTGGCTCGGGCGGTGGTGGGTCGGGTGGCGGCGGATCT (SEQ ID NO: 12).

Single chain Fv polypeptide antibodies can be expressed from a nucleic acid comprising VH-and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have an Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In other embodiments, the antigen binding domain comprises an antibody mimetic protein such as, for example, designed ankyrin repeat protein (DARPin), affibody, adnectin, or anticalin. Constructs with specific binding affinities can be generated using DARPin libraries e.g., as described in Seeger, M.A. et al., Design, construction, and characterization of a second generation DARPin library with reduced hydrophobicity, Protein Sci., 22:1239-1257 (2013).

In some embodiments, the antigen binding domain may be derived from the same species in which the CAR will ultimately be used. For example, for use in humans, the antigen binding domain of the CAR may comprise a human antibody as described elsewhere herein, or a fragment thereof.

“Enriched stereotyped” BCR, or “enriched stereotypy” as it relates to a BCR, refers to BCRs having similar primary structure defined by highly similar Ig V regions in the H and L chains such as, for example, length, amino acid composition, and unique amino acid residues at recombination junctions, and, in some embodiments, sharing distinct H and L CDR3 configurations. These enriched stereotyped BCRs, which can be grouped into different subsets of enriched stereotyped BCR, each conventionally designated by a sequential number (e.g., subset 2, 4 or 6), comprise antigenic targets for the antigen binding domains of the CARs of the present invention. These enriched stereotyped BCRs are directly involved in disease pathogenesis as they have intrinsic ability to signal and activate the B-cell. For example, Stamatopoulos, B. et al., Clin. Cancer Res., 24:5048-5057 (2018), Rovida et al., Clin. Cancer Res., 27(3):729-739 (2021), Hacken, E.T. et al., Leukemia, 33(2):287-298 (2019), Messmer, B.T. et al., J. Exp. Med., 200(4):519-525 (2004) and Rossi, D. & Gaidano, G., Haematologica, 95(12):1992-1995 (2010), each of which is herein incorporated by reference in its entirety, describe multiple distinct sets of enriched stereotyped receptors.

In one embodiment, the subset 1 BCR can be characterized by the use of IGHV1-5-7, IGHD6-19, IGHJ4, and/or IGKV1-39; the subset 2 BCR can be characterized by the use of IGHV3-21, IGHJ6, and/or IGLV3-21; the subset 4 BCR can be characterized by the use of IGHV4-34, IGHD5-5 or D4-17, IGHJ6, and/or IGKV3-30; the subset 5 BCR can be characterized by the use of IGHV1-69, IGHD3-10, IGHJ6, and/or IGKV1-33 or IGL V3-21; the subset 6 BCR can be characterized by the use of IGHV1-69, IGHD3-16, IGHJ3, and/or IGKV3-20; and the subset 8 BCR can be characterized by the use of IGHV4-39, IGHD6-13, IGHJ5, and/or IGKV1-39.

In some embodiments, the enriched stereotyped BCR is a subset 1, 2, 4, 5, 6 or 8 enriched stereotyped BCR. In one embodiment, the antigen binding domain of a CAR of the present invention specifically binds to a subset 1, 2, 4, 5, 6 or 8 enriched stereotyped BCR.

In other embodiments, the enriched stereotyped BCR is a subset 2, 4, or 6 enriched stereotyped BCR. In another embodiment, the antigen binding domain of a CAR of the present invention specifically binds to a subset 2, 4, or 6 enriched stereotyped BCR.

In some embodiments, the enriched stereotyped BCR is characterized as an autonomously active BCR.

In one embodiment, the autonomously active BCR may be driven by autonomous BCR signaling e.g., by HCDR3-mediated binding of an internal epitope of another BCR, effectuating BCR-BCR binding, aggregation, and/or activation (see e.g., Dühren-von Minden, M. et al., Nature, 489(7415):309-12 (2012); see also e.g., US Patent Publication No. 2020/0199225, which is herein incorporated by reference in its entirety).

For example, US Patent Publication No. 2020/0199225 describes the region of a CLL B cell receptor subset 2 variant relevant for the autonomously active functionality of the receptor is characterized by the amino acid sequences KLTVLRQPKA (SEQ ID NO: 13) and VAPGKTAR (SEQ ID NO: 14) of the light chain, while the region of a subset 4 relevant for the autonomously active functionality of the receptor is defined by the amino acid sequences PTIRRYYYYG (SEQ ID NO: 15), NHKPSNTKV (SEQ ID NO: 16), and VSSASTKG (SEQ ID NO:17) of the variable part of the heavy chain.

In other embodiments, the enriched stereotyped BCR comprises the amino acid sequence of KLTVLRQPKA (SEQ ID NO: 13), VAPGKTAR (SEQ ID NO: 14), PTIRRYYYYG (SEQ ID NO: 15), NHKPSNTKV (SEQ ID NO: 16), or VSSASTKG (SEQ ID NO:17).

In another embodiment, the antigen binding domain of the CAR of the present invention specifically binds to a enriched stereotyped BCR comprising a light chain variable region (VL) comprising the amino acid sequence of KLTVLRQPKA (SEQ ID NO: 13) and/or VAPGKTAR (SEQ ID NO: 14).

In another embodiment, the antigen binding domain of the CAR specifically binds to a enriched stereotyped BCR comprising a heavy chain variable region (VH) comprising the amino acid sequence of PTIRRYYYYG (SEQ ID NO: 18) and/or NHKPSNTKV (SEQ ID NO: 19), and/or VSSASTKG (SEQ ID NO: 20).

In another embodiment, the antigen binding domain specifically binds to a enriched stereotyped BCR comprising the sequence of:

(SEQ ID NO: 21)
EVOLVESGGGGGLGLVKPGGSLRLSCAASGFTFRSYSMNWVRQAP
GKGLEWVSSIISSSSYIYADSVKGRFTISRDNAKNSLYLQMNSLR
AEDTALYYCARDQNAMDVWGGTTVTVTVSS;
and/or
(SEQ ID NO: 22)
SYELTQPPSVSVSVSVAPGKTARITCAGNNIGSKSVHWYQQQQAP
VLVIYYDSDRPSGIPERFSGSNSGNTATLTISRVEAGDEADYYCQ
VWDSGSDHPWWVFGGGTKLTVLRQPKAAPSVTLFPPSSEELQANK
ATLVCLISDFYPGAVTVAWKADSSPVKAGVETTTPSKQSNNKYAA
SSYLSLTPEQWKSHRSYSCQVTHEGSTVEKTVAPTECS.

In another embodiment, the antigen binding domain specifically binds to a enriched stereotyped BCR comprising the sequence of:

(SEQ ID NO: 23)
QVQLQQWGAGLLKPSETLSLTCAVYGGSFSGYYWTWIRQSPGKGL
EWIGEINHSGSTTYNPSLKSRVTISVDTSKNQFSLKLNSVTAADT
AVYYCARGYGDTPTIRRYYYYGMDVWGQGTTVTVSSASTKGPSVF
PLAPSSKSTSGGTAALGCLVKDYFPEPVTVSWNSGALTSGVHTFP
ACLQSSGLYSLSSVVTVPSSSLGTQTYICNVNHKPSNTKVDKKC;
and/or
(SEQ ID NO: 24)
DIVMTQSPLSLPVTLGQPASISCRSSQSLVHSDGNTYLNWFQQRP
GQSPRRLIYKVSDRDSGVPDRFSGSGSGTDFTLKISRVEAEDVGL
YYCMQGTHWPPYTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC.

In some embodiments, the antigen binding domain of the CAR of the present invention is a enriched stereotyped BCR-specific binding domain comprising a VH and a VL.

In one embodiment, the antigen binding domain comprises a VH comprising a CDR1, CDR2, and CDR3, wherein the CDR1 comprises the sequence of GFSLTSYG (SEQ ID NO: 25), the CDR2 comprises the sequence of IWRGGGT (SEQ ID NO: 26); and the CDR3 comprises the sequence of ARSRYDEEESMNY (SEQ ID NO: 27).

In one embodiment, the antigen binding domain comprises a VL comprising a CDR1, CDR2, and CDR3, wherein the CDR1 comprises the sequence of GNIHSY (SEQ ID NO: 28), the CDR2 comprises the sequence of NAKT (SEQ ID NO: 29); and the CDR3 comprises the sequence of QHFWNTPPT (SEQ ID NO: 30).

In one embodiment, the antigen binding domain comprises a VH comprising the sequence of:

(SEQ ID NO: 31)
QVQLQQSGPGLVQPSQSLSITCTVSGFSLTSYGIHWVRQSPGKGL
EWLGVIWRGGGTDSNAAFMSRLSITKDNSKSQVFFKMNSLQADDT
AIYYCARSRYDEEESMNYWGQGTSVTVSS;
(SEQ ID NO: 32)
QLVQSGAEVVKPGASVKVSCKASGYTFTSYWMHWVKQAPGQGLEW
IGAVSPGNSDTSYNEKFKGKATLTVDKSASTAYMELSSLRSEDTA
VYYCTRSRYGNNALDYWGQGTLVTVSS;
(SEQ ID NO: 64)
QVHLKESGPGLVQPSQTLSLTCTVSGFSLTFYNVHWVRQPTGKGL
EWMAIIWNGGKTDYNSGLKSRLSISRDTSKSQVFLKMNSLQSEDT
AAYYCVREVGGRFDYWGQGVLVTVSS;
or
(SEQ ID NO: 76)
EVQLQQSGPELEKPGASVKISCKASGYSFTAYNMNWVKQNNGKSL
EWIGNIDPYHGGTNYNQKFKGKATLTVDKSSSTAYMQLKSLTSED
YAVYYCARGGGDWSWFAYWGQGTLVTVSA

In one embodiment, the antigen binding domain comprises a VH encoded by a sequence comprising:

(SEQ ID NO: 33)
CAGGTCCAGTTGCAACAGTCTGGCCCTGGTCTGGTCCAACCTAGT
CAATCTTTGTCCATTACCTGCACTGTGTCTGGTTTTTCTCTCACG
TCTTATGGAATTCATTGGGTTAGACAAAGCCCTGGGAAAGGGTTG
GAGTGGTTGGGTGTGATCTGGCGCGGAGGTGGTACTGACAGCAAC
GCAGCGTTTATGTCTCGGCTTTCCATAACCAAAGACAACAGCAAG
TCTCAGGTGTTCTTCAAGATGAATTCCCTCCAGGCCGACGATACG
GCGATCTATTATTGTGCTCGCTCCAGATACGACGAGGAAGAAAGC
ATGAACTATTGGGGACAGGGGACCTCAGTAACAGTGTCAAGC;
(SEQ ID NO: 34)
CAACTGGTTCAGTCAGGTGCTGAAGTTGTAAAACCAGGCGCTTCT
GTTAAAGTATCCTGTAAAGCTAGCGGATATACATTTACCAGTTAC
TGGATGCACTGGGTAAAGCAGGCTCCAGGACAAGGACTGGAGTGG
ATCGGGGCTGTTAGCCCAGGAAACTCAGATACAAGCTACAACGAG
AAGTTCAAAGGGAAGGCAACTCTGACTGTCGATAAGAGCGCGAGT
ACAGCTTATATGGAACTTTCCTCACTTCGGAGCGAAGACACAGCT
GTATATTATTGTACCAGGTCTCGCTACGGTAATAATGCACTGGAT
TACTGGGGACAAGGAACCCTCGTTACGGTCAGTTCT;
or
(SEQ ID NO: 65)
CAAGTGCACCTCAAAGAATCCGGGCCCGGGCTCGTACAACCGTCC
CAAACACTCAGCCTGACATGTACAGTGTCAGGATTTTCCCTCACG
TTTTATAACGTCCATTGGGTGCGCCAACCAACTGGCAAGGGCCTT
GAATGGATGGCCATCATCTGGAACGGTGGCAAAACGGACTATAAC
AGCGGCCTTAAGAGCAGACTCTCCATATCCCGAGATACTAGCAAA
TCACAGGTGTTTCTCAAGATGAATTCTCTCCAGAGCGAGGATACT
GCTGCCTATTATTGCGTGCGCGAAGTCGGCGGTCGGTTTGACTAT
TGGGGACAGGGCGTTCTCGTGACTGTGAGCAGT;
or
(SEQ ID NO: 77)
GAAGTGCAGCTGCAGCAGAGCGGCCCGGAACTGGAAAAACCGGGC
GCGAGCGTGAAAATTAGCTGCAAAGCGAGCGGCTATAGCTTTACC
GCGTATAACATGAACTGGGTGAAACAGAACAACGGCAAAAGCCTG
GAATGGATTGGCAACATTGATCCGTATCATGGCGGCACCAACTAT
AACCAGAAATTTAAAGGCAAAGCGACCCTGACCGTGGATAAAAGC
AGCAGCACCGCGTATATGCAGCTGAAAAGCCTGACCAGCGAAGAT
TATGCGGTGTATTATTGCGCGCGCGGCGGCGGCGATTGGAGCTGG
TTTGCGTATTGGGGCCAGGGCACCCTGGTGACCGTGAGCGCG.

In one embodiment, the antigen binding domain comprises a VL comprising the sequence of:

(SEQ ID NO: 35)
QIVLTQSPASLSASVGETVTITCRASGNIHSYLAWYQQKQGKSPQ
LLVYNAKTLADGVPSRFSGSGSGTQYSLKINSLQPEDFGSYYCQH
FWNTPPTFGAGTKLELK;
(SEQ ID NO: 36)
DIQLTQSPSSLSASVGDRVTITCRASQGISSNIVWLQQKPGKAPK
GLIYHGTNLESGVPSRFSGSGSGTDYTLTISSLEPEDFATYYCVQ
YSQFPPTFGQGTKLEIK;
(SEQ ID NO: 66)
DFQMTQSPASLSASLGETVTIECLASEDIYDILAWYQQKPGKSPQ
LLIYDASSLHTGVPSRFSGSGSGAQYSLKINSLQPEDFASYYCQS
GLSAPWTFGGGTKLEL;
or
(SEQ ID NO: 78)
DIVMTQSHKFMSTSVGDRVSITCKASQDVNTAVAWYQQKPGQSPK
VLIYWASNRHTGVPDRFTGSGSGTDYTLTISSMQAEDLALYYCHQ
HYSTPWTFGGGTKLEIER.

In one embodiment, the antigen binding domain comprises a VL encoded by a sequence comprising:

(SEQ ID NO: 37)
CAGATCGTTCTTACGCAGAGTCCTGCTTCCCTTTCCGCTTCTGTG
GGCGAAACAGTTACCATTACATGCCGCGCAAGCGGGAATATTCAC
TCTTACCTGGCGTGGTATCAACAAAAGCAAGGTAAATCCCCACAA
CTTCTGGTTTACAACGCCAAAACTCTGGCTGATGGTGTACCTTCA
CGCTTCAGCGGCAGTGGGAGCGGGACTCAGTACTCTCTGAAAATC
AATAGCCTTCAACCAGAAGATTTTGGCTCCTATTATTGCCAACAC
TTTTGGAATACTCCTCCGACTTTCGGGGCTGGGACTAAGCTTGAA
TTGAAA;
(SEQ ID NO: 38)
GATATACAGTTGACGCAGAGTCCATCTTCTCTCAGTGCTAGCGTT
GGAGACCGGGTGACGATTACGTGTAGGGCGAGCCAGGGGATTAGT
TCCAATATCGTGTGGCTCCAACAGAAGCCTGGCAAGGCTCCCAAG
GGCCTTATTTACCACGGGACTAATCTGGAATCAGGTGTACCAAGT
CGATTCTCCGGGTCCGGTTCAGGCACAGACTATACCCTCACGATA
AGTTCATTGGAGCCGGAGGATTTTGCCACATATTATTGCGTCCAG
TATTCACAGTTCCCTCCGACATTTGGTCAGGGGACTAAGCTTGAA
ATTAAG;
(SEQ ID NO: 67)
GATTTTCAAATGACGCAAAGTCCTGCCAGCTTGTCCGCCTCATTG
GGCGAGACAGTTACAATAGAGTGTCTCGCCAGTGAAGATATATAT
GACATCCTGGCCTGGTACCAGCAAAAGCCTGGAAAGTCACCCCAA
CTGCTTATTTACGACGCTTCCTCACTGCACACCGGTGTGCCCTCT
CGTTTTAGCGGCTCCGGCTCAGGTGCCCAATACAGTCTGAAAATT
AACTCACTCCAACCAGAGGACTTCGCATCCTACTATTGCCAGAGC
GGACTCAGCGCTCCATGGACCTTTGGCGGCGGGACGAAATTGGAG
CTG;
or
(SEQ ID NO: 79)
GATATTGTGATGACCCAGAGCCATAAATTTATGAGCACCAGCGTG
GGCGATCGCGTGAGCATTACCTGCAAAGCGAGCCAGGATGTGAAC
ACCGCGGTGGCGTGGTATCAGCAGAAACCGGGCCAGAGCCCGAAA
GTGCTGATTTATTGGGCGAGCAACCGCCATACCGGCGTGCCGGAT
CGCTTTACCGGCAGCGGCAGCGGCACCGATTATACCCTGACCATT
AGCAGCATGCAGGCGGAAGATCTGGCGCTGTATTATTGCCATCAG
CATTATAGCACCCCGTGGACCTTTGGCGGCGGCACCAAACTGGAA
ATTGAACGC.

In some embodiments, the antigen binding domain comprises an scFv having the configuration from N-terminus to C-terminus, VH-linker-VL or VL-linker-VH, wherein the linker comprises the sequence of SEQ ID NO: 11.

In yet other embodiments, the scFv comprises the amino acid sequence of:

(SEQ ID NO: 39)
QVQLQQSGPGLVQPSQSLSITCTVSGFSLTSYGIHWVRQSPGKGL
EWLGVIWRGGGTDSNAAFMSRLSITKDNSKSQVFFKMNSLQADDT
AIYYCARSRYDEEESMNYWGQGTSVTVSSGGGGSGGGGSGGGGSQ
IVLTQSPASLSASVGETVTITCRASGNIHSYLAWYQQKQGKSPQL
LVYNAKTLADGVPSRFSGSGSGTQYSLKINSLQPEDFGSYYCQHF
WNTPPTFGAGTKLELK;
(SEQ ID NO: 40)
QIVLTQSPASLSASVGETVTITCRASGNIHSYLAWYQQKQGKSPQ
LLVYNAKTLADGVPSRFSGSGSGTQYSLKINSLQPEDFGSYYCQH
FWNTPPTFGAGTKLELKGGGGSGGGGSGGGGSQVQLQQSGPGLVQ
PSQSLSITCTVSGFSLTSYGIHWVRQSPGKGLEWLGVIWRGGGTD
SNAAFMSRLSITKDNSKSQVFFKMNSLQADDTAIYYCARSRYDEE
ESMNYWGQGTSVTVSS;
(SEQ ID NO: 41)
DIQLTQSPSSLSASVGDRVTITCRASQGISSNIVWLQQKPGKAPK
GLIYHGTNLESGVPSRFSGSGSGTDYTLTISSLEPEDFATYYCVQ
YSQFPPTFGQGTKLEIKGGGGSGGGGSGGGGSQLVQSGAEVVKPG
ASVKVSCKASGYTFTSYWMHWVKQAPGQGLEWIGAVSPGNSDTSY
NEKFKGKATLTVDKSASTAYMELSSLRSEDTAVYYCTRSRYGNNA
LDYWGQGTLVTVSS;
(SEQ ID NO: 42)
QLVQSGAEVVKPGASVKVSCKASGYTFTSYWMHWVKQAPGQGLEW
IGAVSPGNSDTSYNEKFKGKATLTVDKSASTAYMELSSLRSEDTA
VYYCTRSRYGNNALDYWGQGTLVTVSSGGGGSGGGGSGGGGSDIQ
LTQSPSSLSASVGDRVTITCRASQGISSNIVWLQQKPGKAPKGLI
YHGTNLESGVPSRFSGSGSGTDYTLTISSLEPEDFATYYCVQYSQ
FPPTFGQGTKLEIK;
(SEQ ID NO: 68)
QVHLKESGPGLVQPSQTLSLTCTVSGFSLTFYNVHWVRQPTGKGL
EWMAIIWNGGKTDYNSGLKSRLSISRDTSKSQVFLKMNSLQSEDT
AAYYCVREVGGRFDYWGQGVLVTVSSGGGGSGGGGSGGGGSDFQM
TQSPASLSASLGETVTIECLASEDIYDILAWYQQKPGKSPQLLIY
DASSLHTGVPSRFSGSGSGAQYSLKINSLQPEDFASYYCQSGLSA
PWTFGGGTKLEL;
(SEQ ID NO: 69)
DFQMTQSPASLSASLGETVTIECLASEDIYDILAWYQQKPGKSPQ
LLIYDASSLHTGVPSRFSGSGSGAQYSLKINSLQPEDFASYYCQS
GLSAPWTFGGGTKLELGGGGSGGGGSGGGGSQVHLKESGPGLVQP
SQTLSLTCTVSGFSLTFYNVHWVRQPTGKGLEWMAIIWNGGKTDY
NSGLKSRLSISRDTSKSQVFLKMNSLQSEDTAAYYCVREVGGRFD
YWGQGVLVTVSS;
(SEQ ID NO: 80)
EVQLQQSGPELEKPGASVKISCKASGYSFTAYNMNWVKQNNGKSL
EWIGNIDPYHGGTNYNQKFKGKATLTVDKSSSTAYMQLKSLTSED
YAVYYCARGGGDWSWFAYWGQGTLVTVSAGGGGSGGGGSGGGGSD
IVMTQSHKFMSTSVGDRVSITCKASQDVNTAVAWYQQKPGQSPKV
LIYWASNRHTGVPDRFTGSGSGTDYTLTISSMQAEDLALYYCHQH
YSTPWTFGGGTKLEIER;
or
(SEQ ID NO: 81)
DIVMTQSHKFMSTSVGDRVSITCKASQDVNTAVAWYQQKPGQSPK
VLIYWASNRHTGVPDRFTGSGSGTDYTLTISSMQAEDLALYYCHQ
HYSTPWTFGGGTKLEIERGGGGSGGGGSGGGGSEVQLQQSGPELE
KPGASVKISCKASGYSFTAYNMNWVKQNNGKSLEWIGNIDPYHGG
TNYNQKFKGKATLTVDKSSSTAYMQLKSLTSEDYAVYYCARGGGD
WSWFAYWGQGTLVTVSA.

In further embodiments, the scFv is encoded by a nucleic acid sequence comprising:

(SEQ ID NO: 43)
CAGGTCCAGTTGCAACAGTCTGGCCCTGGTCTGGTCCAACCTAGT
CAATCTTTGTCCATTACCTGCACTGTGTCTGGTTTTTCTCTCACG
TCTTATGGAATTCATTGGGTTAGACAAAGCCCTGGGAAAGGGTTG
GAGTGGTTGGGTGTGATCTGGCGCGGAGGTGGTACTGACAGCAAC
GCAGCGTTTATGTCTCGGCTTTCCATAACCAAAGACAACAGCAAG
TCTCAGGTGTTCTTCAAGATGAATTCCCTCCAGGCCGACGATACG
GCGATCTATTATTGTGCTCGCTCCAGATACGACGAGGAAGAAAGC
ATGAACTATTGGGGACAGGGGACCTCAGTAACAGTGTCAAGCGGT
GGTGGAGGGAGTGGAGGGGGTGGGTCTGGTGGTGGTGGTAGCCAG
ATCGTTCTTACGCAGAGTCCTGCTTCCCTTTCCGCTTCTGTGGGC
GAAACAGTTACCATTACATGCCGCGCAAGCGGGAATATTCACTCT
TACCTGGCGTGGTATCAACAAAAGCAAGGTAAATCCCCACAACTT
CTGGTTTACAACGCCAAAACTCTGGCTGATGGTGTACCTTCACGC
TTCAGCGGCAGTGGGAGCGGGACTCAGTACTCTCTGAAAATCAAT
AGCCTTCAACCAGAAGATTTTGGCTCCTATTATTGCCAACACTTT
TGGAATACTCCTCCGACTTTCGGGGCTGGGACTAAGCTTGAATTG
AAA;
(SEQ ID NO: 44)
CAAATCGTGCTCACTCAGAGCCCAGCCTCTTTGTCCGCTTCCGTG
GGCGAAACTGTCACCATAACCTGCCGCGCCAGTGGTAATATACAC
AGCTACCTGGCATGGTATCAACAAAAGCAAGGTAAATCTCCACAA
CTTCTTGTATACAACGCCAAAACACTGGCCGATGGAGTTCCCTCT
CGTTTCAGCGGCAGCGGAAGTGGGACTCAGTACTCCCTGAAAATC
AATTCCTTGCAACCTGAAGATTTTGGTTCCTATTATTGCCAACAC
TTTTGGAATACCCCGCCAACATTCGGAGCTGGCACTAAGTTGGAG
CTGAAAGGAGGAGGCGGATCAGGCGGGGGGGGTTCCGGTGGTGGT
GGCTCTCAGGTCCAACTGCAGCAGTCCGGCCCTGGTCTGGTCCAA
CCTTCCCAAAGTCTGTCCATAACATGCACAGTGTCAGGGTTTTCA
TTGACGTCTTATGGAATACATTGGGTTAGGCAAAGTCCTGGAAAA
GGCCTCGAATGGCTTGGGGTGATCTGGCGCGGAGGAGGCACAGAC
AGCAACGCGGCTTTTATGAGTCGTCTCTCAATTACTAAAGACAAC
TCTAAGTCACAGGTGTTCTTCAAGATGAATAGTCTCCAGGCGGAC
GATACAGCTATCTATTATTGTGCTCGCTCTCGCTACGACGAGGAA
GAAAGCATGAACTATTGGGGACAGGGCACCTCTGTAACAGTGTCA
AGC;
(SEQ ID NO: 45)
GATATACAGTTGACGCAGAGTCCATCTTCTCTCAGTGCTAGCGTT
GGAGACCGGGTGACGATTACGTGTAGGGCGAGCCAGGGGATTAGT
TCCAATATCGTGTGGCTCCAACAGAAGCCTGGCAAGGCTCCCAAG
GGCCTTATTTACCACGGGACTAATCTGGAATCAGGTGTACCAAGT
CGATTCTCCGGGTCCGGTTCAGGCACAGACTATACCCTCACGATA
AGTTCATTGGAGCCGGAGGATTTTGCCACATATTATTGCGTCCAG
TATTCACAGTTCCCTCCGACATTTGGTCAGGGGACTAAGCTTGAA
ATTAAGGGCGGAGGTGGCTCCGGTGGTGGGGGCTCAGGCGGCGGG
GGCTCTCAACTGGTTCAGTCAGGTGCTGAAGTTGTAAAACCAGGC
GCTTCTGTTAAAGTATCCTGTAAAGCTAGCGGATATACATTTACC
AGTTACTGGATGCACTGGGTAAAGCAGGCTCCAGGACAAGGACTG
GAGTGGATCGGGGCTGTTAGCCCAGGAAACTCAGATACAAGCTAC
AACGAGAAGTTCAAAGGGAAGGCAACTCTGACTGTCGATAAGAGC
GCGAGTACAGCTTATATGGAACTTTCCTCACTTCGGAGCGAAGAC
ACAGCTGTATATTATTGTACCAGGTCTCGCTACGGTAATAATGCA
CTGGATTACTGGGGACAAGGAACCCTCGTTACGGTCAGTTCT;
(SEQ ID NO: 46)
CAACTGGTTCAGTCAGGTGCTGAAGTTGTAAAACCAGGCGCTTCT
GTTAAAGTATCCTGTAAAGCTAGCGGATATACATTTACCAGTTAC
TGGATGCACTGGGTAAAGCAGGCTCCAGGACAAGGACTGGAGTGG
ATCGGGGCTGTTAGCCCAGGAAACTCAGATACAAGCTACAACGAG
AAGTTCAAAGGGAAGGCAACTCTGACTGTCGATAAGAGCGCGAGT
ACAGCTTATATGGAACTTTCCTCACTTCGGAGCGAAGACACAGCT
GTATATTATTGTACCAGGTCTCGCTACGGTAATAATGCACTGGAT
TACTGGGGACAAGGAACCCTCGTTACGGTCAGTTCTGGCGGAGGT
GGCTCCGGTGGTGGGGGCTCAGGCGGCGGGGGCTCTGATATACAG
TTGACGCAGAGTCCATCTTCTCTCAGTGCTAGCGTTGGAGACCGG
GTGACGATTACGTGTAGGGCGAGCCAGGGGATTAGTTCCAATATC
GTGTGGCTCCAACAGAAGCCTGGCAAGGCTCCCAAGGGCCTTATT
TACCACGGGACTAATCTGGAATCAGGTGTACCAAGTCGATTCTCC
GGGTCCGGTTCAGGCACAGACTATACCCTCACGATAAGTTCATTG
GAGCCGGAGGATTTTGCCACATATTATTGCGTCCAGTATTCACAG
TTCCCTCCGACATTTGGTCAGGGGACTAAGCTTGAAATTAAG;
(SEQ ID NO: 70)
CAAGTGCACCTCAAAGAATCCGGGCCCGGGCTCGTACAACCGTCC
CAAACACTCAGCCTGACATGTACAGTGTCAGGATTTTCCCTCACG
TTTTATAACGTCCATTGGGTGCGCCAACCAACTGGCAAGGGCCTT
GAATGGATGGCCATCATCTGGAACGGTGGCAAAACGGACTATAAC
AGCGGCCTTAAGAGCAGACTCTCCATATCCCGAGATACTAGCAAA
TCACAGGTGTTTCTCAAGATGAATTCTCTCCAGAGCGAGGATACT
GCTGCCTATTATTGCGTGCGCGAAGTCGGCGGTCGGTTTGACTAT
TGGGGACAGGGCGTTCTCGTGACTGTGAGCAGTGGCGGGGGGGGC
TCAGGAGGAGGTGGCTCTGGCGGGGGGGGGTCTGATTTTCAAATG
ACGCAAAGTCCTGCCAGCTTGTCCGCCTCATTGGGCGAGACAGTT
ACAATAGAGTGTCTCGCCAGTGAAGATATATATGACATCCTGGCC
TGGTACCAGCAAAAGCCTGGAAAGTCACCCCAACTGCTTATTTAC
GACGCTTCCTCACTGCACACCGGTGTGCCCTCTCGTTTTAGCGGC
TCCGGCTCAGGTGCCCAATACAGTCTGAAAATTAACTCACTCCAA
CCAGAGGACTTCGCATCCTACTATTGCCAGAGCGGACTCAGCGCT
CCATGGACCTTTGGCGGCGGGACGAAATTGGAGCTG;
(SEQ ID NO: 71)
GATTTTCAAATGACCCAAAGCCCTGCATCACTTAGCGCCAGTCTC
GGCGAGACCGTGACTATAGAGTGTCTGGCCTCAGAAGATATCTAT
GACATCCTTGCTTGGTACCAGCAGAAACCCGGCAAGAGCCCCCAA
CTGCTCATATACGACGCATCCTCTCTGCACACGGGCGTTCCTTCC
CGCTTTAGCGGGTCTGGTAGCGGGGCGCAATACTCCCTGAAAATT
AACAGTCTTCAACCCGAGGACTTCGCGTCCTACTATTGCCAGAGC
GGTCTTAGCGCTCCATGGACATTTGGTGGCGGAACGAAACTTGAG
CTGGGCGGTGGTGGCAGTGGTGGTGGTGGTTCAGGCGGCGGCGGG
TCACAAGTTCACCTCAAAGAAAGTGGCCCAGGCCTCGTCCAACCA
AGCCAAACACTCTCACTGACATGTACGGTGAGCGGATTTTCACTC
ACATTTTATAACGTTCATTGGGTGCGCCAACCCACGGGGAAGGGA
CTTGAATGGATGGCTATTATTTGGAACGGTGGTAAAACTGACTAT
AACAGTGGACTTAAGAGTAGGCTCAGTATTTCACGCGATACAAGT
AAATCTCAGGTGTTTCTTAAGATGAATTCCTTGCAGTCCGAGGAT
ACCGCTGCCTATTATTGCGTGCGAGAGGTCGGTGGCCGCTTCGAC
TATTGGGGTCAGGGAGTGCTCGTAACGGTGAGCAGC;
(SEQ ID NO: 82)
GAAGTGCAGCTGCAGCAGAGCGGCCCGGAACTGGAAAAACCGGGC
GCGAGCGTGAAAATTAGCTGCAAAGCGAGCGGCTATAGCTTTACC
GCGTATAACATGAACTGGGTGAAACAGAACAACGGCAAAAGCCTG
GAATGGATTGGCAACATTGATCCGTATCATGGCGGCACCAACTAT
AACCAGAAATTTAAAGGCAAAGCGACCCTGACCGTGGATAAAAGC
AGCAGCACCGCGTATATGCAGCTGAAAAGCCTGACCAGCGAAGAT
TATGCGGTGTATTATTGCGCGCGCGGCGGCGGCGATTGGAGCTGG
TTTGCGTATTGGGGCCAGGGCACCCTGGTGACCGTGAGCGCGGGC
GGCGGCGGCAGCGGCGGCGGCGGCAGCGGCGGCGGCGGCAGCGAT
ATTGTGATGACCCAGAGCCATAAATTTATGAGCACCAGCGTGGGC
GATCGCGTGAGCATTACCTGCAAAGCGAGCCAGGATGTGAACACC
GCGGTGGCGTGGTATCAGCAGAAACCGGGCCAGAGCCCGAAAGTG
CTGATTTATTGGGCGAGCAACCGCCATACCGGCGTGCCGGATCGC
TTTACCGGCAGCGGCAGCGGCACCGATTATACCCTGACCATTAGC
AGCATGCAGGCGGAAGATCTGGCGCTGTATTATTGCCATCAGCAT
TATAGCACCCCGTGGACCTTTGGCGGCGGCACCAAACTGGAAATT
GAACGC;
or
(SEQ ID NO: 83)
GATATTGTGATGACCCAGAGCCATAAATTTATGAGCACCAGCGTG
GGCGATCGCGTGAGCATTACCTGCAAAGCGAGCCAGGATGTGAAC
ACCGCGGTGGCGTGGTATCAGCAGAAACCGGGCCAGAGCCCGAAA
GTGCTGATTTATTGGGCGAGCAACCGCCATACCGGCGTGCCGGAT
CGCTTTACCGGCAGCGGCAGCGGCACCGATTATACCCTGACCATT
AGCAGCATGCAGGCGGAAGATCTGGCGCTGTATTATTGCCATCAG
CATTATAGCACCCCGTGGACCTTTGGCGGCGGCACCAAACTGGAA
ATTGAACGCGGCGGCGGCGGCAGCGGCGGCGGCGGCAGCGGCGGC
GGCGGCAGCGAAGTGCAGCTGCAGCAGAGCGGCCCGGAACTGGAA
AAACCGGGCGCGAGCGTGAAAATTAGCTGCAAAGCGAGCGGCTAT
AGCTTTACCGCGTATAACATGAACTGGGTGAAACAGAACAACGGC
AAAAGCCTGGAATGGATTGGCAACATTGATCCGTATCATGGCGGC
ACCAACTATAACCAGAAATTTAAAGGCAAAGCGACCCTGACCGTG
GATAAAAGCAGCAGCACCGCGTATATGCAGCTGAAAAGCCTGACC
AGCGAAGATTATGCGGTGTATTATTGCGCGCGCGGCGGCGGCGAT
TGGAGCTGGTTTGCGTATTGGGGCCAGGGCACCCTGGTGACCGTG
AGCGCG.

Tolerable variations of the antigen binding domain will be known to those of skill in the art, while maintaining specific binding to the enriched stereotyped BCR. For example, in some embodiments the antigen binding domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of the amino acid sequences set forth in SEQ ID NOs: 25-30, 31-32, 35-36, 39-42, 64, 66, 68, 69, 80, and 81.

In other embodiments the antigen binding domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of the nucleic acid sequences set forth in SEQ ID NOs: 33-34, 37-38, 43-46, 65, 67, 70, 71, 82, and 83.

The antigen binding domain may be operably linked to another domain of the CAR, such as the transmembrane domain or the intracellular domain, both described elsewhere herein. In one embodiment, a nucleic acid encoding the antigen binding domain is operably linked to a nucleic acid encoding a transmembrane domain and a nucleic acid encoding an intracellular domain.

The antigen binding domains described herein, such as the antibody or fragment thereof that specifically binds to the enriched stereotyped BCR can be combined with any of the transmembrane domains described herein, any of the intracellular domains or cytoplasmic domains described herein, or any of the other domains described herein that may be included in the CAR.

For example, in some embodiments, the CAR comprises the sequence of:

(SEQ ID NO: 47)
MALPVTALLLPLALLLHAARPGSQIVLTQSPASLSASVGETVTIT
CRASGNIHSYLAWYQQKQGKSPQLLVYNAKTLADGVPSRFSGSGS
GTQYSLKINSLQPEDFGSYYCQHFWNTPPTFGAGTKLELKGGGGS
GGGGSGGGGSQVQLQQSGPGLVQPSQSLSITCTVSGFSLTSYGIH
WVRQSPGKGLEWLGVIWRGGGTDSNAAFMSRLSITKDNSKSQVFF
KMNSLQADDTAIYYCARSRYDEEESMNYWGQGTSVTVSSSGTTTP
APRPPTPAPTIASQPLSLRPEACRPAAGGAVHTRGLDFACDIYIW
APLAGTCGVLLLSLVITLYCKRGRKKLLYIFKQPFMRPVQTTQEE
DGCSCRFPEEEEGGCELRVKFSRSADAPAYKQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPR;
(SEQ ID NO: 48)
MALPVTALLLPLALLLHAARPGSQVQLQQSGPGLVQPSQSLSITC
TVSGFSLTSYGIHWVRQSPGKGLEWLGVIWRGGGTDSNAAFMSRL
SITKDNSKSQVFFKMNSLQADDTAIYYCARSRYDEEESMNYWGQG
TSVTVSSGGGGSGGGGSGGGGSQIVLTQSPASLSASVGETVTITC
RASGNIHSYLAWYQQKQGKSPQLLVYNAKTLADGVPSRFSGSGSG
TQYSLKINSLQPEDFGSYYCQHFWNTPPTFGAGTKLELKSGTTTP
APRPPTPAPTIASQPLSLRPEACRPAAGGAVHTRGLDFACDIYIW
APLAGTCGVLLLSLVITLYCKRGRKKLLYIFKQPFMRPVQTTQEE
DGCSCRFPEEEEGGCELRVKFSRSADAPAYKQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPR;
(SEQ ID NO: 49)
MALPVTALLLPLALLLHAARPGSDIQLTQSPSSLSASVGDRVTIT
CRASQGISSNIVWLQQKPGKAPKGLIYHGTNLESGVPSRFSGSGS
GTDYTLTISSLEPEDFATYYCVQYSQFPPTFGQGTKLEIKGGGGS
GGGGSGGGGSQLVQSGAEVVKPGASVKVSCKASGYTFTSYWMHWV
KQAPGQGLEWIGAVSPGNSDTSYNEKFKGKATLTVDKSASTAYME
LSSLRSEDTAVYYCTRSRYGNNALDYWGQGTLVTVSSSGTTTPAP
RPPTPAPTIASQPLSLRPEACRPAAGGAVHTRGLDFACDIYIWAP
LAGTCGVLLLSLVITLYCKRGRKKLLYIFKQPFMRPVQTTQEEDG
CSCRFPEEEEGGCELRVKFSRSADAPAYKQGQNQLYNELNLGRRE
EYDVLDKRRGRDPEMGGKPRRKNPQEGLYNELQKDKMAEAYSEIG
MKGERRRGKGHDGLYQGLSTATKDTYDALHMQALPPR;
(SEQ ID NO: 50)
MALPVTALLLPLALLLHAARPGSQLVQSGAEVVKPGASVKVSCKA
SGYTFTSYWMHWVKQAPGQGLEWIGAVSPGNSDTSYNEKFKGKAT
LTVDKSASTAYMELSSLRSEDTAVYYCTRSRYGNNALDYWGQGTL
VTVSSGGGGSGGGGSGGGGSDIQLTQSPSSLSASVGDRVTITCRA
SQGISSNIVWLQQKPGKAPKGLIYHGTNLESGVPSRFSGSGSGTD
YTLTISSLEPEDFATYYCVQYSQFPPTFGQGTKLEIKSGTTTPAP
RPPTPAPTIASQPLSLRPEACRPAAGGAVHTRGLDFACDIYIWAP
LAGTCGVLLLSLVITLYCKRGRKKLLYIFKQPFMRPVQTTQEEDG
CSCRFPEEEEGGCELRVKFSRSADAPAYKQGQNQLYNELNLGRRE
EYDVLDKRRGRDPEMGGKPRRKNPQEGLYNELQKDKMAEAYSEIG
MKGERRRGKGHDGLYQGLSTATKDTYDALHMQALPPR;
(SEQ ID NO: 72)
MALPVTALLLPLALLLHAARPGSQVHLKESGPGLVQPSQTLSLTC
TVSGFSLTFYNVHWVRQPTGKGLEWMAIIWNGGKTDYNSGLKSRL
SISRDTSKSQVFLKMNSLQSEDTAAYYCVREVGGRFDYWGQGVLV
TVSSGGGGSGGGGSGGGGSDFQMTQSPASLSASLGETVTIECLAS
EDIYDILAWYQQKPGKSPQLLIYDASSLHTGVPSRFSGSGSGAQY
SLKINSLQPEDFASYYCQSGLSAPWTFGGGTKLELSGTTTPAPRP
PTPAPTIASQPLSLRPEACRPAAGGAVHTRGLDFACDIYIWAPLA
GTCGVLLLSLVITLYCKRGRKKLLYIFKQPFMRPVQTTQEEDGCS
CRFPEEEEGGCELRVKFSRSADAPAYKQGQNQLYNELNLGRREEY
DVLDKRRGRDPEMGGKPRRKNPQEGLYNELQKDKMAEAYSEIGMK
GERRRGKGHDGLYQGLSTATKDTYDALHMQALPPR;
(SEQ ID NO: 73)
MALPVTALLLPLALLLHAARPGSDFQMTQSPASLSASLGETVTIE
CLASEDIYDILAWYQQKPGKSPQLLIYDASSLHTGVPSRFSGSGS
GAQYSLKINSLQPEDFASYYCQSGLSAPWTFGGGTKLELGGGGSG
GGGSGGGGSQVHLKESGPGLVQPSQTLSLTCTVSGFSLTFYNVHW
VRQPTGKGLEWMAIIWNGGKTDYNSGLKSRLSISRDTSKSQVFLK
MNSLQSEDTAAYYCVREVGGRFDYWGQGVLVTVSSSGTTTPAPRP
PTPAPTIASQPLSLRPEACRPAAGGAVHTRGLDFACDIYIWAPLA
GTCGVLLLSLVITLYCKRGRKKLLYIFKQPFMRPVQTTQEEDGCS
CRFPEEEEGGCELRVKFSRSADAPAYKQGQNQLYNELNLGRREEY
DVLDKRRGRDPEMGGKPRRKNPQEGLYNELQKDKMAEAYSEIGMK
GERRRGKGHDGLYQGLSTATKDTYDALHMQALPPR;
(SEQ ID NO: 74)
MALPVTALLLPLALLLHAARPGSEVQLQQSGPELEKPGASVKISC
KASGYSFTAYNMNWVKQNNGKSLEWIGNIDPYHGGTNYNQKFKGK
ATLTVDKSSSTAYMQLKSLTSEDYAVYYCARGGGDWSWFAYWGQG
TLVTVSAGGGGSGGGGSGGGGSDIVMTQSHKFMSTSVGDRVSITC
KASQDVNTAVAWYQQKPGQSPKVLIYWASNRHTGVPDRFTGSGSG
TDYTLTISSMQAEDLALYYCHQHYSTPWTFGGGTKLEIERSGTTT
PAPRPPTPAPTIASQPLSLRPEACRPAAGGAVHTRGLDFACDIYI
WAPLAGTCGVLLLSLVITLYCKRGRKKLLYIFKQPFMRPVQTTQE
EDGCSCRFPEEEEGGCELRVKFSRSADAPAYKQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR;
or
(SEQ ID NO: 75)
MALPVTALLLPLALLLHAARPGSDIVMTQSHKFMSTSVGDRVSIT
CKASQDVNTAVAWYQQKPGQSPKVLIYWASNRHTGVPDRFTGSGS
GTDYTLTISSMQAEDLALYYCHQHYSTPWTFGGGTKLEIERGGGG
SGGGGSGGGGSEVQLQQSGPELEKPGASVKISCKASGYSFTAYNM
NWVKQNNGKSLEWIGNIDPYHGGTNYNQKFKGKATLTVDKSSSTA
YMQLKSLTSEDYAVYYCARGGGDWSWFAYWGQGTLVTVSASGTTT
PAPRPPTPAPTIASQPLSLRPEACRPAAGGAVHTRGLDFACDIYI
WAPLAGTCGVLLLSLVITLYCKRGRKKLLYIFKQPFMRPVQTTQE
EDGCSCRFPEEEEGGCELRVKFSRSADAPAYKQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR.

In some embodiments the CAR comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of the amino acid sequences set forth in SEQ ID NOs: 47-50 and 72-75.

In other embodiments, the CAR is encoded by a nucleic acid sequence comprising:

(SEQ ID NO: 84)
ATGGCGCTGCCGGTGACCGCGCTGCTGCTGCCGCTGGCGCTGCTG
CTGCATGCGGCGCGCCCGGGCAGCGAAGTGCAGCTGCAGCAGAGC
GGCCCGGAACTGGAAAAACCGGGCGCGAGCGTGAAAATTAGCTGC
AAAGCGAGCGGCTATAGCTTTACCGCGTATAACATGAACTGGGTG
AAACAGAACAACGGCAAAAGCCTGGAATGGATTGGCAACATTGAT
CCGTATCATGGCGGCACCAACTATAACCAGAAATTTAAAGGCAAA
GCGACCCTGACCGTGGATAAAAGCAGCAGCACCGCGTATATGCAG
CTGAAAAGCCTGACCAGCGAAGATTATGCGGTGTATTATTGCGCG
CGCGGCGGCGGCGATTGGAGCTGGTTTGCGTATTGGGGCCAGGGC
ACCCTGGTGACCGTGAGCGCGGGCGGCGGCGGCAGCGGCGGCGGC
GGCAGCGGCGGCGGCGGCAGCGATATTGTGATGACCCAGAGCCAT
AAATTTATGAGCACCAGCGTGGGCGATCGCGTGAGCATTACCTGC
AAAGCGAGCCAGGATGTGAACACCGCGGTGGCGTGGTATCAGCAG
AAACCGGGCCAGAGCCCGAAAGTGCTGATTTATTGGGCGAGCAAC
CGCCATACCGGCGTGCCGGATCGCTTTACCGGCAGCGGCAGCGGC
ACCGATTATACCCTGACCATTAGCAGCATGCAGGCGGAAGATCTG
GCGCTGTATTATTGCCATCAGCATTATAGCACCCCGTGGACCTTT
GGCGGCGGCACCAAACTGGAAATTGAACGCAGCGGCACCACCACC
CCGGCGCCGCGCCCGCCGACCCCGGCGCCGACCATTGCGAGCCAG
CCGCTGAGCCTGCGCCCGGAAGCGTGCCGCCCGGCGGCGGGCGGC
GCGGTGCATACCCGCGGCCTGGATTTTGCGTGCGATATTTATATT
TGGGCGCCGCTGGCGGGCACCTGCGGCGTGCTGCTGCTGAGCCTG
GTGATTACCCTGTATTGCAAACGCGGCCGCAAAAAACTGCTGTAT
ATTTTTAAACAGCCGTTTATGCGCCCGGTGCAGACCACCCAGGAA
GAAGATGGCTGCAGCTGCCGCTTTCCGGAAGAAGAAGAAGGCGGC
TGCGAACTGCGCGTGAAATTTAGCCGCAGCGCGGATGCGCCGGCG
TATAAACAGGGCCAGAACCAGCTGTATAACGAACTGAACCTGGGC
CGCCGCGAAGAATATGATGTGCTGGATAAACGCCGCGGCCGCGAT
CCGGAAATGGGCGGCAAACCGCGCCGCAAAAACCCGCAGGAAGGC
CTGTATAACGAACTGCAGAAAGATAAAATGGCGGAAGCGTATAGC
GAAATTGGCATGAAAGGCGAACGCCGCCGCGGCAAAGGCCATGAT
GGCCTGTATCAGGGCCTGAGCACCGCGACCAAAGATACCTATGAT
GCGCTGCATATGCAGGCGCTGCCGCCGCGC;
(SEQ ID NO: 85)
ATGGCGCTGCCGGTGACCGCGCTGCTGCTGCCGCTGGCGCTGCTG
CTGCATGCGGCGCGCCCGGGCAGCCAGGTGCAGCTGCAGCAGAGC
GGCCCGGGCCTGGTGCAGCCGAGCCAGAGCCTGAGCATTACCTGC
ACCGTGAGCGGCTTTAGCCTGACCAGCTATGGCATTCATTGGGTG
CGCCAGAGCCCGGGCAAAGGCCTGGAATGGCTGGGCGTGATTTGG
CGCGGCGGCGGCACCGATAGCAACGCGGCGTTTATGAGCCGCCTG
AGCATTACCAAAGATAACAGCAAAAGCCAGGTGTTTTTTAAAATG
AACAGCCTGCAGGCGGATGATACCGCGATTTATTATTGCGCGCGC
AGCCGCTATGATGAAGAAGAAAGCATGAACTATTGGGGCCAGGGC
ACCAGCGTGACCGTGAGCAGCGGCGGCGGCGGCAGCGGCGGCGGC
GGCAGCGGCGGCGGCGGCAGCCAGATTGTGCTGACCCAGAGCCCG
GCGAGCCTGAGCGCGAGCGTGGGCGAAACCGTGACCATTACCTGC
CGCGCGAGCGGCAACATTCATAGCTATCTGGCGTGGTATCAGCAG
AAACAGGGCAAAAGCCCGCAGCTGCTGGTGTATAACGCGAAAACC
CTGGCGGATGGCGTGCCGAGCCGCTTTAGCGGCAGCGGCAGCGGC
ACCCAGTATAGCCTGAAAATTAACAGCCTGCAGCCGGAAGATTTT
GGCAGCTATTATTGCCAGCATTTTTGGAACACCCCGCCGACCTTT
GGCGCGGGCACCAAACTGGAACTGAAAAGCGGCACCACCACCCCG
GCGCCGCGCCCGCCGACCCCGGCGCCGACCATTGCGAGCCAGCCG
CTGAGCCTGCGCCCGGAAGCGTGCCGCCCGGCGGCGGGCGGCGCG
GTGCATACCCGCGGCCTGGATTTTGCGTGCGATATTTATATTTGG
GCGCCGCTGGCGGGCACCTGCGGCGTGCTGCTGCTGAGCCTGGTG
ATTACCCTGTATTGCAAACGCGGCCGCAAAAAACTGCTGTATATT
TTTAAACAGCCGTTTATGCGCCCGGTGCAGACCACCCAGGAAGAA
GATGGCTGCAGCTGCCGCTTTCCGGAAGAAGAAGAAGGCGGCTGC
GAACTGCGCGTGAAATTTAGCCGCAGCGCGGATGCGCCGGCGTAT
AAACAGGGCCAGAACCAGCTGTATAACGAACTGAACCTGGGCCGC
CGCGAAGAATATGATGTGCTGGATAAACGCCGCGGCCGCGATCCG
GAAATGGGCGGCAAACCGCGCCGCAAAAACCCGCAGGAAGGCCTG
TATAACGAACTGCAGAAAGATAAAATGGCGGAAGCGTATAGCGAA
ATTGGCATGAAAGGCGAACGCCGCCGCGGCAAAGGCCATGATGGC
CTGTATCAGGGCCTGAGCACCGCGACCAAAGATACCTATGATGCG
CTGCATATGCAGGCGCTGCCGCCGCGC;
(SEQ ID NO: 86)
ATGGCGCTGCCGGTGACCGCGCTGCTGCTGCCGCTGGCGCTGCTG
CTGCATGCGGCGCGCCCGGGCAGCGATATTCAGCTGACCCAGAGC
CCGAGCAGCCTGAGCGCGAGCGTGGGCGATCGCGTGACCATTACC
TGCCGCGCGAGCCAGGGCATTAGCAGCAACATTGTGTGGCTGCAG
CAGAAACCGGGCAAAGCGCCGAAAGGCCTGATTTATCATGGCACC
AACCTGGAAAGCGGCGTGCCGAGCCGCTTTAGCGGCAGCGGCAGC
GGCACCGATTATACCCTGACCATTAGCAGCCTGGAACCGGAAGAT
TTTGCGACCTATTATTGCGTGCAGTATAGCCAGTTTCCGCCGACC
TTTGGCCAGGGCACCAAACTGGAAATTAAAGGCGGCGGCGGCAGC
GGCGGCGGCGGCAGCGGCGGCGGCGGCAGCCAGCTGGTGCAGAGC
GGCGCGGAAGTGGTGAAACCGGGCGCGAGCGTGAAAGTGAGCTGC
AAAGCGAGCGGCTATACCTTTACCAGCTATTGGATGCATTGGGTG
AAACAGGCGCCGGGCCAGGGCCTGGAATGGATTGGCGCGGTGAGC
CCGGGCAACAGCGATACCAGCTATAACGAAAAATTTAAAGGCAAA
GCGACCCTGACCGTGGATAAAAGCGCGAGCACCGCGTATATGGAA
CTGAGCAGCCTGCGCAGCGAAGATACCGCGGTGTATTATTGCACC
CGCAGCCGCTATGGCAACAACGCGCTGGATTATTGGGGCCAGGGC
ACCCTGGTGACCGTGAGCAGCAGCGGCACCACCACCCCGGCGCCG
CGCCCGCCGACCCCGGCGCCGACCATTGCGAGCCAGCCGCTGAGC
CTGCGCCCGGAAGCGTGCCGCCCGGCGGCGGGCGGCGCGGTGCAT
ACCCGCGGCCTGGATTTTGCGTGCGATATTTATATTTGGGCGCCG
CTGGCGGGCACCTGCGGCGTGCTGCTGCTGAGCCTGGTGATTACC
CTGTATTGCAAACGCGGCCGCAAAAAACTGCTGTATATTTTTAAA
CAGCCGTTTATGCGCCCGGTGCAGACCACCCAGGAAGAAGATGGC
TGCAGCTGCCGCTTTCCGGAAGAAGAAGAAGGCGGCTGCGAACTG
CGCGTGAAATTTAGCCGCAGCGCGGATGCGCCGGCGTATAAACAG
GGCCAGAACCAGCTGTATAACGAACTGAACCTGGGCCGCCGCGAA
GAATATGATGTGCTGGATAAACGCCGCGGCCGCGATCCGGAAATG
GGCGGCAAACCGCGCCGCAAAAACCCGCAGGAAGGCCTGTATAAC
GAACTGCAGAAAGATAAAATGGCGGAAGCGTATAGCGAAATTGGC
ATGAAAGGCGAACGCCGCCGCGGCAAAGGCCATGATGGCCTGTAT
CAGGGCCTGAGCACCGCGACCAAAGATACCTATGATGCGCTGCAT
ATGCAGGCGCTGCCGCCGCGC;
(SEQ ID NO: 87)
ATGGCGCTGCCGGTGACCGCGCTGCTGCTGCCGCTGGCGCTGCTG
CTGCATGCGGCGCGCCCGGGCAGCCAGCTGGTGCAGAGCGGCGCG
GAAGTGGTGAAACCGGGCGCGAGCGTGAAAGTGAGCTGCAAAGCG
AGCGGCTATACCTTTACCAGCTATTGGATGCATTGGGTGAAACAG
GCGCCGGGCCAGGGCCTGGAATGGATTGGCGCGGTGAGCCCGGGC
AACAGCGATACCAGCTATAACGAAAAATTTAAAGGCAAAGCGACC
CTGACCGTGGATAAAAGCGCGAGCACCGCGTATATGGAACTGAGC
AGCCTGCGCAGCGAAGATACCGCGGTGTATTATTGCACCCGCAGC
CGCTATGGCAACAACGCGCTGGATTATTGGGGCCAGGGCACCCTG
GTGACCGTGAGCAGCGGCGGCGGCGGCAGCGGCGGCGGCGGCAGC
GGCGGCGGCGGCAGCGATATTCAGCTGACCCAGAGCCCGAGCAGC
CTGAGCGCGAGCGTGGGCGATCGCGTGACCATTACCTGCCGCGCG
AGCCAGGGCATTAGCAGCAACATTGTGTGGCTGCAGCAGAAACCG
GGCAAAGCGCCGAAAGGCCTGATTTATCATGGCACCAACCTGGAA
AGCGGCGTGCCGAGCCGCTTTAGCGGCAGCGGCAGCGGCACCGAT
TATACCCTGACCATTAGCAGCCTGGAACCGGAAGATTTTGCGACC
TATTATTGCGTGCAGTATAGCCAGTTTCCGCCGACCTTTGGCCAG
GGCACCAAACTGGAAATTAAAAGCGGCACCACCACCCCGGCGCCG
CGCCCGCCGACCCCGGCGCCGACCATTGCGAGCCAGCCGCTGAGC
CTGCGCCCGGAAGCGTGCCGCCCGGCGGCGGGCGGCGCGGTGCAT
ACCCGCGGCCTGGATTTTGCGTGCGATATTTATATTTGGGCGCCG
CTGGCGGGCACCTGCGGCGTGCTGCTGCTGAGCCTGGTGATTACC
CTGTATTGCAAACGCGGCCGCAAAAAACTGCTGTATATTTTTAAA
CAGCCGTTTATGCGCCCGGTGCAGACCACCCAGGAAGAAGATGGC
TGCAGCTGCCGCTTTCCGGAAGAAGAAGAAGGCGGCTGCGAACTG
CGCGTGAAATTTAGCCGCAGCGCGGATGCGCCGGCGTATAAACAG
GGCCAGAACCAGCTGTATAACGAACTGAACCTGGGCCGCCGCGAA
GAATATGATGTGCTGGATAAACGCCGCGGCCGCGATCCGGAAATG
GGCGGCAAACCGCGCCGCAAAAACCCGCAGGAAGGCCTGTATAAC
GAACTGCAGAAAGATAAAATGGCGGAAGCGTATAGCGAAATTGGC
ATGAAAGGCGAACGCCGCCGCGGCAAAGGCCATGATGGCCTGTAT
CAGGGCCTGAGCACCGCGACCAAAGATACCTATGATGCGCTGCAT
ATGCAGGCGCTGCCGCCGCGC;
(SEQ ID NO: 88)
ATGGCGCTGCCGGTGACCGCGCTGCTGCTGCCGCTGGCGCTGCTG
CTGCATGCGGCGCGCCCGGGCAGCCAGGTGCATCTGAAAGAAAGC
GGCCCGGGCCTGGTGCAGCCGAGCCAGACCCTGAGCCTGACCTGC
ACCGTGAGCGGCTTTAGCCTGACCTTTTATAACGTGCATTGGGTG
CGCCAGCCGACCGGCAAAGGCCTGGAATGGATGGCGATTATTTGG
AACGGCGGCAAAACCGATTATAACAGCGGCCTGAAAAGCCGCCTG
AGCATTAGCCGCGATACCAGCAAAAGCCAGGTGTTTCTGAAAATG
AACAGCCTGCAGAGCGAAGATACCGCGGCGTATTATTGCGTGCGC
GAAGTGGGCGGCCGCTTTGATTATTGGGGCCAGGGCGTGCTGGTG
ACCGTGAGCAGCGGCGGCGGCGGCAGCGGCGGCGGCGGCAGCGGC
GGCGGCGGCAGCGATTTTCAGATGACCCAGAGCCCGGCGAGCCTG
AGCGCGAGCCTGGGCGAAACCGTGACCATTGAATGCCTGGCGAGC
GAAGATATTTATGATATTCTGGCGTGGTATCAGCAGAAACCGGGC
AAAAGCCCGCAGCTGCTGATTTATGATGCGAGCAGCCTGCATACC
GGCGTGCCGAGCCGCTTTAGCGGCAGCGGCAGCGGCGCGCAGTAT
AGCCTGAAAATTAACAGCCTGCAGCCGGAAGATTTTGCGAGCTAT
TATTGCCAGAGCGGCCTGAGCGCGCCGTGGACCTTTGGCGGCGGC
ACCAAACTGGAACTGAGCGGCACCACCACCCCGGCGCCGCGCCCG
CCGACCCCGGCGCCGACCATTGCGAGCCAGCCGCTGAGCCTGCGC
CCGGAAGCGTGCCGCCCGGCGGCGGGCGGCGCGGTGCATACCCGC
GGCCTGGATTTTGCGTGCGATATTTATATTTGGGCGCCGCTGGCG
GGCACCTGCGGCGTGCTGCTGCTGAGCCTGGTGATTACCCTGTAT
TGCAAACGCGGCCGCAAAAAACTGCTGTATATTTTTAAACAGCCG
TTTATGCGCCCGGTGCAGACCACCCAGGAAGAAGATGGCTGCAGC
TGCCGCTTTCCGGAAGAAGAAGAAGGCGGCTGCGAACTGCGCGTG
AAATTTAGCCGCAGCGCGGATGCGCCGGCGTATAAACAGGGCCAG
AACCAGCTGTATAACGAACTGAACCTGGGCCGCCGCGAAGAATAT
GATGTGCTGGATAAACGCCGCGGCCGCGATCCGGAAATGGGCGGC
AAACCGCGCCGCAAAAACCCGCAGGAAGGCCTGTATAACGAACTG
CAGAAAGATAAAATGGCGGAAGCGTATAGCGAAATTGGCATGAAA
GGCGAACGCCGCCGCGGCAAAGGCCATGATGGCCTGTATCAGGGC
CTGAGCACCGCGACCAAAGATACCTATGATGCGCTGCATATGCAG
GCGCTGCCGCCGCGC;
(SEQ ID NO: 89)
ATGGCGCTGCCGGTGACCGCGCTGCTGCTGCCGCTGGCGCTGCTG
CTGCATGCGGCGCGCCCGGGCAGCGATTTTCAGATGACCCAGAGC
CCGGCGAGCCTGAGCGCGAGCCTGGGCGAAACCGTGACCATTGAA
TGCCTGGCGAGCGAAGATATTTATGATATTCTGGCGTGGTATCAG
CAGAAACCGGGCAAAAGCCCGCAGCTGCTGATTTATGATGCGAGC
AGCCTGCATACCGGCGTGCCGAGCCGCTTTAGCGGCAGCGGCAGC
GGCGCGCAGTATAGCCTGAAAATTAACAGCCTGCAGCCGGAAGAT
TTTGCGAGCTATTATTGCCAGAGCGGCCTGAGCGCGCCGTGGACC
TTTGGCGGCGGCACCAAACTGGAACTGGGCGGCGGCGGCAGCGGC
GGCGGCGGCAGCGGCGGCGGCGGCAGCCAGGTGCATCTGAAAGAA
AGCGGCCCGGGCCTGGTGCAGCCGAGCCAGACCCTGAGCCTGACC
TGCACCGTGAGCGGCTTTAGCCTGACCTTTTATAACGTGCATTGG
GTGCGCCAGCCGACCGGCAAAGGCCTGGAATGGATGGCGATTATT
TGGAACGGCGGCAAAACCGATTATAACAGCGGCCTGAAAAGCCGC
CTGAGCATTAGCCGCGATACCAGCAAAAGCCAGGTGTTTCTGAAA
ATGAACAGCCTGCAGAGCGAAGATACCGCGGCGTATTATTGCGTG
CGCGAAGTGGGCGGCCGCTTTGATTATTGGGGCCAGGGCGTGCTG
GTGACCGTGAGCAGCAGCGGCACCACCACCCCGGCGCCGCGCCCG
CCGACCCCGGCGCCGACCATTGCGAGCCAGCCGCTGAGCCTGCGC
CCGGAAGCGTGCCGCCCGGCGGCGGGCGGCGCGGTGCATACCCGC
GGCCTGGATTTTGCGTGCGATATTTATATTTGGGCGCCGCTGGCG
GGCACCTGCGGCGTGCTGCTGCTGAGCCTGGTGATTACCCTGTAT
TGCAAACGCGGCCGCAAAAAACTGCTGTATATTTTTAAACAGCCG
TTTATGCGCCCGGTGCAGACCACCCAGGAAGAAGATGGCTGCAGC
TGCCGCTTTCCGGAAGAAGAAGAAGGCGGCTGCGAACTGCGCGTG
AAATTTAGCCGCAGCGCGGATGCGCCGGCGTATAAACAGGGCCAG
AACCAGCTGTATAACGAACTGAACCTGGGCCGCCGCGAAGAATAT
GATGTGCTGGATAAACGCCGCGGCCGCGATCCGGAAATGGGCGGC
AAACCGCGCCGCAAAAACCCGCAGGAAGGCCTGTATAACGAACTG
CAGAAAGATAAAATGGCGGAAGCGTATAGCGAAATTGGCATGAAA
GGCGAACGCCGCCGCGGCAAAGGCCATGATGGCCTGTATCAGGGC
CTGAGCACCGCGACCAAAGATACCTATGATGCGCTGCATATGCAG
GCGCTGCCGCCGCGC;
(SEQ ID NO: 90)
ATGGCGCTGCCGGTGACCGCGCTGCTGCTGCCGCTGGCGCTGCTG
CTGCATGCGGCGCGCCCGGGCAGCGAAGTGCAGCTGCAGCAGAGC
GGCCCGGAACTGGAAAAACCGGGCGCGAGCGTGAAAATTAGCTGC
AAAGCGAGCGGCTATAGCTTTACCGCGTATAACATGAACTGGGTG
AAACAGAACAACGGCAAAAGCCTGGAATGGATTGGCAACATTGAT
CCGTATCATGGCGGCACCAACTATAACCAGAAATTTAAAGGCAAA
GCGACCCTGACCGTGGATAAAAGCAGCAGCACCGCGTATATGCAG
CTGAAAAGCCTGACCAGCGAAGATTATGCGGTGTATTATTGCGCG
CGCGGCGGCGGCGATTGGAGCTGGTTTGCGTATTGGGGCCAGGGC
ACCCTGGTGACCGTGAGCGCGGGCGGCGGCGGCAGCGGCGGCGGC
GGCAGCGGCGGCGGCGGCAGCGATATTGTGATGACCCAGAGCCAT
AAATTTATGAGCACCAGCGTGGGCGATCGCGTGAGCATTACCTGC
AAAGCGAGCCAGGATGTGAACACCGCGGTGGCGTGGTATCAGCAG
AAACCGGGCCAGAGCCCGAAAGTGCTGATTTATTGGGCGAGCAAC
CGCCATACCGGCGTGCCGGATCGCTTTACCGGCAGCGGCAGCGGC
ACCGATTATACCCTGACCATTAGCAGCATGCAGGCGGAAGATCTG
GCGCTGTATTATTGCCATCAGCATTATAGCACCCCGTGGACCTTT
GGCGGCGGCACCAAACTGGAAATTGAACGCAGCGGCACCACCACC
CCGGCGCCGCGCCCGCCGACCCCGGCGCCGACCATTGCGAGCCAG
CCGCTGAGCCTGCGCCCGGAAGCGTGCCGCCCGGCGGCGGGCGGC
GCGGTGCATACCCGCGGCCTGGATTTTGCGTGCGATATTTATATT
TGGGCGCCGCTGGCGGGCACCTGCGGCGTGCTGCTGCTGAGCCTG
GTGATTACCCTGTATTGCAAACGCGGCCGCAAAAAACTGCTGTAT
ATTTTTAAACAGCCGTTTATGCGCCCGGTGCAGACCACCCAGGAA
GAAGATGGCTGCAGCTGCCGCTTTCCGGAAGAAGAAGAAGGCGGC
TGCGAACTGCGCGTGAAATTTAGCCGCAGCGCGGATGCGCCGGCG
TATAAACAGGGCCAGAACCAGCTGTATAACGAACTGAACCTGGGC
CGCCGCGAAGAATATGATGTGCTGGATAAACGCCGCGGCCGCGAT
CCGGAAATGGGCGGCAAACCGCGCCGCAAAAACCCGCAGGAAGGC
CTGTATAACGAACTGCAGAAAGATAAAATGGCGGAAGCGTATAGC
GAAATTGGCATGAAAGGCGAACGCCGCCGCGGCAAAGGCCATGAT
GGCCTGTATCAGGGCCTGAGCACCGCGACCAAAGATACCTATGAT
GCGCTGCATATGCAGGCGCTGCCGCCGCGC;
or
(SEQ ID NO: 91)
ATGGCGCTGCCGGTGACCGCGCTGCTGCTGCCGCTGGCGCTGCTG
CTGCATGCGGCGCGCCCGGGCAGCGATATTGTGATGACCCAGAGC
CATAAATTTATGAGCACCAGCGTGGGCGATCGCGTGAGCATTACC
TGCAAAGCGAGCCAGGATGTGAACACCGCGGTGGCGTGGTATCAG
CAGAAACCGGGCCAGAGCCCGAAAGTGCTGATTTATTGGGCGAGC
AACCGCCATACCGGCGTGCCGGATCGCTTTACCGGCAGCGGCAGC
GGCACCGATTATACCCTGACCATTAGCAGCATGCAGGCGGAAGAT
CTGGCGCTGTATTATTGCCATCAGCATTATAGCACCCCGTGGACC
TTTGGCGGCGGCACCAAACTGGAAATTGAACGCGGCGGCGGCGGC
AGCGGCGGCGGCGGCAGCGGCGGCGGCGGCAGCGAAGTGCAGCTG
CAGCAGAGCGGCCCGGAACTGGAAAAACCGGGCGCGAGCGTGAAA
ATTAGCTGCAAAGCGAGCGGCTATAGCTTTACCGCGTATAACATG
AACTGGGTGAAACAGAACAACGGCAAAAGCCTGGAATGGATTGGC
AACATTGATCCGTATCATGGCGGCACCAACTATAACCAGAAATTT
AAAGGCAAAGCGACCCTGACCGTGGATAAAAGCAGCAGCACCGCG
TATATGCAGCTGAAAAGCCTGACCAGCGAAGATTATGCGGTGTAT
TATTGCGCGCGCGGCGGCGGCGATTGGAGCTGGTTTGCGTATTGG
GGCCAGGGCACCCTGGTGACCGTGAGCGCGAGCGGCACCACCACC
CCGGCGCCGCGCCCGCCGACCCCGGCGCCGACCATTGCGAGCCAG
CCGCTGAGCCTGCGCCCGGAAGCGTGCCGCCCGGCGGCGGGCGGC
GCGGTGCATACCCGCGGCCTGGATTTTGCGTGCGATATTTATATT
TGGGCGCCGCTGGCGGGCACCTGCGGCGTGCTGCTGCTGAGCCTG
GTGATTACCCTGTATTGCAAACGCGGCCGCAAAAAACTGCTGTAT
ATTTTTAAACAGCCGTTTATGCGCCCGGTGCAGACCACCCAGGAA
GAAGATGGCTGCAGCTGCCGCTTTCCGGAAGAAGAAGAAGGCGGC
TGCGAACTGCGCGTGAAATTTAGCCGCAGCGCGGATGCGCCGGCG
TATAAACAGGGCCAGAACCAGCTGTATAACGAACTGAACCTGGGC
CGCCGCGAAGAATATGATGTGCTGGATAAACGCCGCGGCCGCGAT
CCGGAAATGGGCGGCAAACCGCGCCGCAAAAACCCGCAGGAAGGC
CTGTATAACGAACTGCAGAAAGATAAAATGGCGGAAGCGTATAGC
GAAATTGGCATGAAAGGCGAACGCCGCCGCGGCAAAGGCCATGAT
GGCCTGTATCAGGGCCTGAGCACCGCGACCAAAGATACCTATGAT
GCGCTGCATATGCAGGCGCTGCCGCCGCGC.

In some embodiments, the CAR is encoded by a nucleic acid sequence comprising an nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of the sequences set forth in SEQ ID NOs: 84-91.

### Transmembrane Domain

In some embodiments, the CAR comprises a transmembrane domain that is fused to the extracellular domain. In one embodiment, the CAR comprises a transmembrane domain that naturally is associated with one of the domains of the CAR. In some embodiments, the transmembrane domain is selected or modified by amino acid substitution to avoid binding to the transmembrane domains of the same or different surface membrane proteins in order to minimize interactions with other members of the receptor complex.

The transmembrane domain may be derived either from a natural or from a synthetic source. When the source is natural, the domain may be derived from any membrane-bound or transmembrane protein. In one embodiment, the transmembrane domain may be synthetic, in which case it will comprise predominantly hydrophobic residues such as leucine and valine. In one aspect a triplet of phenylalanine, tryptophan and valine will be found at each end of a synthetic transmembrane domain. Optionally, a short oligo- or polypeptide linker, between 2 and 10 amino acids in length may form the linkage between the transmembrane domain and the cytoplasmic signaling domain of the CAR. A glycine-serine (GS) doublet provides a particularly suitable linker.

In other embodiments, between the extracellular domain and the transmembrane domain of the CAR, or between the cytoplasmic domain and the transmembrane domain of the CAR, there may be incorporated a spacer domain. As used herein, the term “spacer domain” generally refers to any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

In some embodiments, a spacer domain before the transmembrane domain can be employed including a CD8 or human Ig (immunoglobulin) hinge, or a glycine-serine linker. In one embodiment, a hinge (e.g., a CD8 alpha hinge) that is N-terminal to the transmembrane domain is included in the CAR between the extracellular domain and the transmembrane domain of the CAR e.g., between the antigen binding domain and the transmembrane domain of the CAR.

Examples of the hinge and/or transmembrane domain include, but are not limited to, a hinge and/or transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIR, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In one embodiment, the CAR comprises a transmembrane domain, such as, but not limited to, CD8 alpha transmembrane domain comprising the sequence of: IYIWAPLAGTCGVLLLSLVITLYC (SEQ ID NO: 51) or an amino acid sequence having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In some embodiments, the CD8 alpha transmembrane domain is encoded by a sequence comprising:

ATCTACATCTGGGCACCCTTGGCTGGAACATGCGGGGTCCTGCTGCTGAGCTT GGTGATCACCCTTTACTGC (SEQ ID NO: 52),

ATCTACATCTGGGCGCCCTTGGCCGGGACTTGTGGGGTCCTTCTCCTGTCACT GGTTATCACCCTTTACTGC (SEQ ID NO: 53), or a nucleic acid sequence having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 60 or 61.

In some embodiments, the CAR comprises a CD8 alpha hinge comprising the sequence of: TTTPAPRPPTPAPTIASQPLSLRPEACRPAAGGAVHTRGLDFACD (SEQ ID NO: 54) or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In some embodiments, the CD8 alpha hinge region is encoded by a sequence comprising: ACCACGACGCCAGCACCGCGACCACCAACACCTGCGCCCACCATCGCGTCGCAGCC CCTGTCCCTGCGCCCAGAGGCGTGCAGACCAGCAGCGGGGGGCGCAGTGCACACGA GGGGGCTGGACTTCGCCTGTGAT (SEQ ID NO: 55) or a nucleic acid sequence having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

### Cytoplasmic Domain

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. In some embodiments, the cytoplasmic signaling molecule in the CAR of the invention comprises a cytoplasmic signaling sequence derived from CD3 zeta (CD35).

In one embodiment, the cytoplasmic signaling sequence derived from CD3 zeta comprises the amino acid sequence of:

RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 56), or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In another embodiment, the nucleic acid sequence encoding the CD3 zeta signaling domain comprises the sequence of:

AGAGTAAAGTTCAGTAGGTCCGCCGATGCCCCAGCCTATCAACAGGGGCAAA ATCAACTCTACAACGAACTTAATCTGGGACGCCGAGAGGAGTACGATGTCTTGGAT AAGAGACGCGGCAGGGACCCTGAAATGGGCGGAAAGCCAAGACGGAAGAACCCCC AGGAAGGTCTGTACAATGAACTTCAGAAAGATAAGATGGCCGAAGCCTACAGCGAG ATCGGCATGAAAGGAGAGAGGCGCCGCGGCAAAGGGCATGATGGACTGTATCAGG GTCTCAGTACTGCTACTAAGGACACATATGATGCCCTCCACATGCAGGCCCTGCCAC CAAGGTGA (SEQ ID NO: 57) or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto; or

AGAGTGAAGTTCAGCAGGAGCGCAGACGCCCCCGCGTACCAGCAGGGCCAG AACCAGCTCTATAACGAGCTCAATCTAGGACGAAGAGAGGAGTACGATGTTTTGGA CAAGAGACGTGGCCGGGACCCTGAGATGGGGGGAAAGCCGAGAAGGAAGAACCCT CAGGAAGGCCTGTACAATGAACTGCAGAAAGATAAGATGGCGGAGGCCTACAGTGA GATTGGGATGAAAGGCGAGCGCCGGAGGGGCAAGGGGCACGATGGCCTTTACCAG GGTCTCAGTACAGCCACCAAGGACACCTACGACGCCCTTCACATGCAGGCCCTGCCC CCTCGC (SEQ ID NO: 58) or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In another embodiment, the cytoplasmic domain of the CAR comprises the CD3 zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signaling element, other costimulatory elements are within the scope of the invention.

In one embodiment, the intracellular signaling domain of 4-1BB comprises the amino acid sequence of: KRGRKKLLYIFKQPFMRPVQTTQEEDGCSCRFPEEEEGGCEL (SEQ ID NO: 59), or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In another embodiment, the nucleic acid sequence encoding the intracellular signaling domain of 4-1BB comprises the sequence of:

AAACGGGGCAGAAAGAAACTCCTGTATATATTCAAACAACCATTTATGAGAC CAGTACAAACTACTCAAGAGGAAGATGGCTGTAGCTGCCGATTTCCAGAAGAAGAA GAAGGAGGATGTGAACTG (SEQ ID NO: 60) or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

The cytoplasmic signaling sequences within the cytoplasmic signaling portion of the CAR of the invention may be linked to each other in a random or specified order. Optionally, a short oligo-or polypeptide linker, preferably between 2 and 10 amino acids in length may form the linkage. A glycine-serine doublet provides a particularly suitable linker.

In one embodiment, the cytoplasmic domain comprises the signaling domain of CD3-zeta and the intracellular signaling domain of 4-1BB. In another embodiment, the cytoplasmic domain comprises the signaling domain of CD3-zeta and the signaling domain of CD28.

In one embodiment, the signaling domain of CD28 comprises the amino acid sequence of: RSKRSRLLHSDYMNMTPRRPGPTRKHYQPYAPPRDFAAYRS (SEQ ID NO: 61), or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In another embodiment, the nucleic acid sequence encoding the signaling domain of CD28 comprises the sequence of:

AGGAGTAAGAGGAGCAGGCTCCTGCACAGTGACTACATGAACATGACTCCCC GCCGCCCCGGGCCCACCCGCAAGCATTACCAGCCCTATGCCCCACCACGCGACTTCG CAGCCTATCGCTCC (SEQ ID NO: 62) or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

In yet another embodiment, the cytoplasmic domain comprises the signaling domain of CD3-zeta and the intracellular signaling domain of 4-1BB and CD28.

In one embodiment, the cytoplasmic domain in the CAR of the invention comprises the intracellular signaling domain of 4-1BB and the signaling domain of CD3-zeta, wherein the intracellular signaling domain of 4-1BB comprises the amino acid sequence set forth in SEQ ID NO: 59 and the signaling domain of CD3-zeta comprises the amino acid sequence set forth in SEQ ID NO: 56.

### Other Domains

In some embodiments, the CAR comprise a signal peptide (e.g., CD8 signal peptide). In some embodiments, the signal peptide is responsible for the translocation of the receptor to the T cell surface.

In some embodiments, signal peptide is a CD8 signal peptide comprising the amino acid sequence of MALPVTALLLPLALLLHAARP (SEQ ID NO: 63) or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto.

### Nucleic Acids/Vectors

In other aspects, the present invention provides recombinant nucleic acid molecules comprising sequences encoding the CAR of the invention having an antigen binding domain that specifically binds to the enriched stereotyped BCR.

The nucleic acid sequences coding for the desired molecules can be obtained using recombinant methods known in the art, such as, for example by screening libraries from cells expressing the gene, by deriving the gene from a vector known to include the same, by screening against a pathogenic target (e.g., ADAMTS13), or by isolating directly from cells and tissues containing the same, using standard techniques. Alternatively, the gene of interest can be produced synthetically, rather than cloned.

The present invention also provides, in some embodiments, vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In other embodiments, a CAR is introduced into cells (e.g., T cells) using, for example, a transposon system (e.g., PiggyBac™), liposomes, nanoparticles, lipid nanoparticles, or mRNAs. In still other embodiments, a CAR of the present invention is introduced into cells (e.g., T cells) in vivo.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. In other embodiments, a CAR is introduced into cells (e.g., T cells) using, for example, a transposon system (e.g., PiggyBac™), liposomes, nanoparticles, lipid nanoparticles, or mRNAs. In still other embodiments, a CAR of the present invention is introduced into cells (e.g., T cells) in vivo.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Sources of T Cells

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, placenta tissues, tissue from a site of infection, ascites, pleural effusion, spleen tissue, tumors, and through the differentiation of T cells from induced pluripotent stem cells (iPSCs) or other stem cell sources. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours, and can go up to 14 days. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4^{°} cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° Celsius per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample, or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

Nonactivated T cells or T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8^{°}). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of Tc cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

In other aspects, the present invention encompasses a cell (e.g., T cell) transduced with a lentiviral vector (LV) or other suitable vector. For example, some embodiments, the LV encodes the CAR of the present invention comprising the antigen binding domain, the transmembrane domain, and the intracellular signaling domain, wherein the antigen binding domain specifically binds to the enriched stereotyped BCR. Therefore, in other embodiments, the transduced T cell can elicit a CAR-mediated T-cell response against specific malignant and/or pathogenic B-cell clones in e.g., lymphoma, leukemia or autoimmune disease.

The invention provides the use of a CAR to redirect the specificity of an immune cell (e.g., a primary T cell) to an antigen present on the enriched stereotyped BCR. Thus, the present invention also provides a method for stimulating a T cell-mediated immune response to a target B cell population in a mammal comprising the step of administering to the mammal a T cell that expresses a CAR of the present invention.

In one embodiment, the present invention includes a type of cellular therapy where immune cells (e.g., T cells) are genetically modified to express a CAR (e.g., CAR against VL3-21, VH3-23 and VH1-69 VH4-34,) and the CAR immune cell (e.g., CAR T cell) is infused to a recipient in need thereof. The infused cell is able to target enriched stereotyped B cells in the recipient e.g., targeting malignant or pathogenic B-cell clones in e.g., lymphoma, leukemia or autoimmune disease while preserving normal B-cells. In some embodiments, unlike antibody therapies, CAR immune cells (e.g., CAR T cells) are able to replicate in vivo resulting in long-term persistence that can lead to sustained tumor control.

In one embodiment, the CAR T cells of the invention can undergo robust in vivo T cell expansion and can persist for an extended amount of time. In another embodiment, the CAR T cells of the invention evolve into specific memory T cells that can be reactivated to inhibit any additional tumor formation or growth. Without wishing to be bound by any particular theory, CAR T cells may differentiate in vivo into a central memory-like state upon encounter and subsequent elimination of target cells expressing the surrogate antigen.

Cancers that may be treated or prevented include hematologic cancers such as cancers of the blood or bone marrow. Examples of hematological (or hematogenous) cancers include leukemias, including, but not limited to, acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In some embodiments, the cancer is chronic lymphocytic leukemia (CLL), mantle cell lymphoma (MCL), diffuse large B-cell lymphoma (DLBCL), or splenic marginal zone lymphoma (SMZL).

In other embodiments, the CAR cells (e.g., CAR T cells) of the present invention are used, alone or in combination with other treatments, to reduce or eliminate antigen escape by pathogenic B cells (e.g., cancerous B cells of CLL patients). Accordingly, in one embodiment, the present invention provides a method for reducing or eliminating antigen escape by a B cell cancer in a subject in need thereof, the method comprising administering to the subject an effective amount of a genetically modified cell disclosed herein. In some embodiments, the B cell cancer is a CD19 negative cancer. In other embodiments, the B cell cancer is a CD19 negative cancer.

Several specific BCRs (e.g., VH 4-34, 1-69 and others) are also enriched in autoimmune diseases: e.g., systemic lupus erythematosus, Crohn's disease, Behcet's disease, Eosinophilic granulomatosis with polyangiitis, and Thrombotic thrombocytopenia purpura (TTP). See e.g., Bashford-Rogers, R. J. M. et al., Nature, 574 (7776): 122-1261-29 (2019) and Ostertag, E. M. et al., Transfusion 56:1763-1774 (2016), each of which is herein incorporated by reference in its entirety. In some embodiment, CAR T cells can be developed for patients with B cell mediated autoimmune diseases. In other embodiments, an advantage of this approach as compared to other approaches for autoimmune diseases (e.g., CAART) is that the present approach uses a CAR comprising an antigen binding domain (e.g., an scFv) rather than the Ab target protein.

In some embodiments, autoimmune diseases that may be treated or prevented include, but are not limited to, systemic lupus erythematosus (SLE), Crohn's disease (CD), Behcet's disease (BD), eosinophilic granulomatosis with polyangiitis (EGPA), thrombotic thrombocytopeni purpura (TTP), ANCA-associated vasculitis (AAV), IgA vasculitis (IgAV), and IgA vasculitis (IgAV).

The CAR-modified T cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an anti-cancer effective amount”, “a cancer-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities. In further embodiments, the T cells of the invention may be used in combination with plasmapheresis, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Anti-IGLV3-21 R110, Anti-IGHV1-69, and Anti-IGHV4-34 BCR CAR T Cells Specifically Kill Lymphoma Cells Expressing VL3-21 R110, VH1-69, and VH4-34 BCR, Respectively

Jekol cells were generated that were depleted of their endogenous BCR, and these were transduced with an appropriate vector in order to express the individual enriched stereotyped BCRs: VL3-21 R110 BCR (“Jekol VL3-21*”), VH1-69 (“Jekol VH1-69”), or VH4-34 (“Jekol VH4-34”). Jekol cells with their endogenous BCR (“Jekol WT BCR”) and CD19 knock-out cells (“Jekol CD19KO”) served as controls.

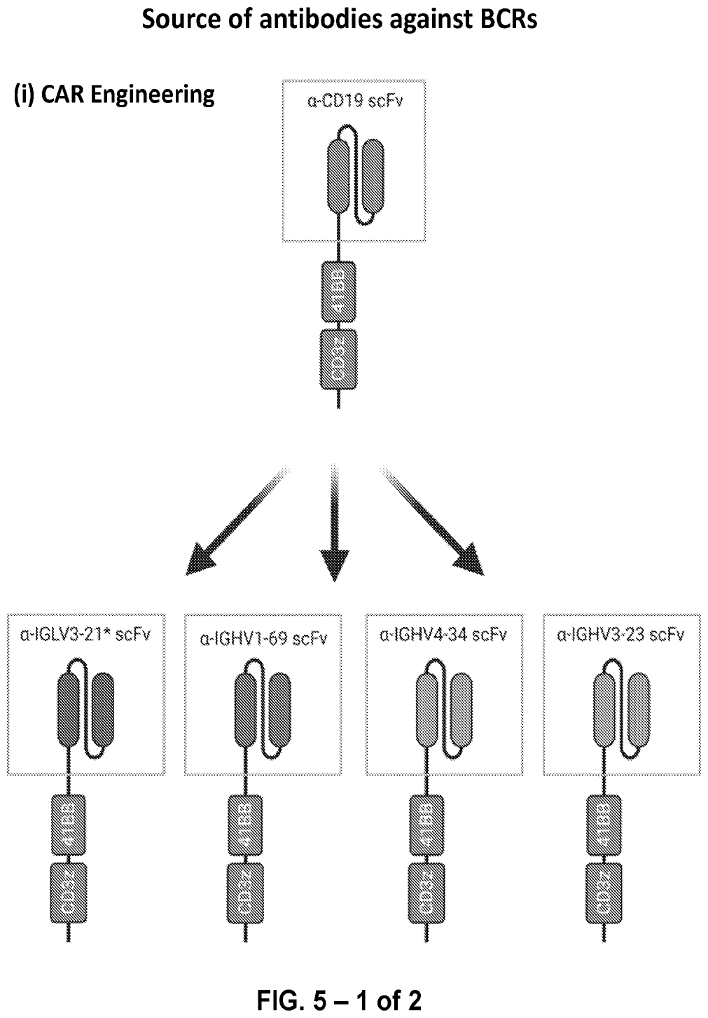

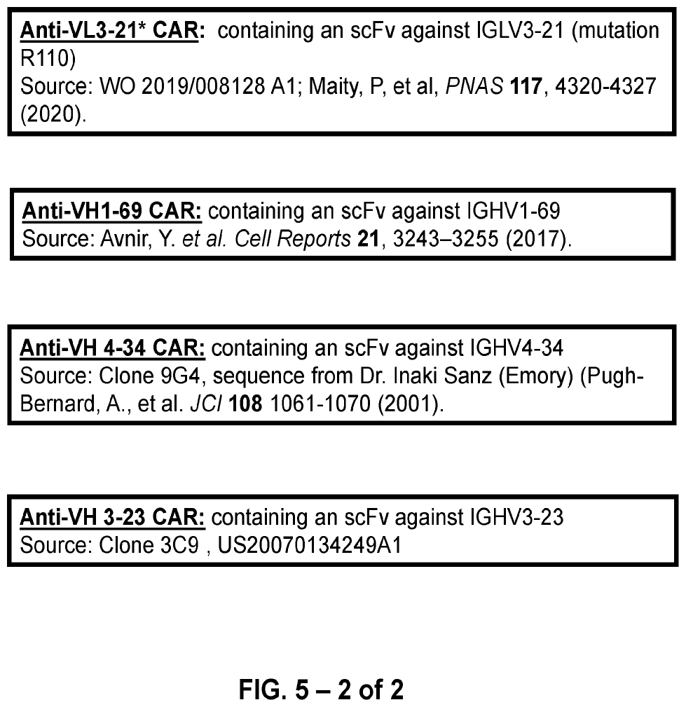

Anti-IGLV3-21 R110 BCR CAR T cells (“CART3-21*”) were prepared by transducing T cells with the pTRPE AVA L2H CAR vector depicted in FIG. 5 to express a CAR anti-IGLV3-21 R110 BCR CAR-T cells) comprising the amino acid sequence of SEQ ID NO: 47, which includes an scFv (SEQ ID NO: 35) that specifically binds to VL3-21 R110, a CD8 hinge (SEQ ID NO: 54), a CD8 transmembrane domain (SEQ ID NO: 51), an intracellular domain (SEQ ID NO: 59) of the costimulatory molecule 4-1 BB, and a CD3 zeta signaling domain (SEQ ID NO: 56).

In order to prepare anti-IGHV1-69 BCR CAR T cells (“CART1-69”), the sequence encoding the CAR of vector pTRPE AVA L2H CAR was replaced with the sequence encoding the CAR of SEQ ID NO: 50, and transduced into T cells to obtain anti-IGHV1-69 BCR CAR-T cells (“CART1-69”).

In order to prepare anti-IGHV4-34 BCR CAR T cells (“CART4-34”), the sequence encoding the CAR of vector pTRPE AVA L2H CAR was replaced with the sequence encoding the CAR of SEQ ID NO: X, and transduced into T cells to obtain anti-IGHV4-34 BCR CAR-T cells (“CART4-34”).

In order to prepare anti-IGHV3-23 BCR CAR T cells (“CART3-23”), the sequence encoding the CAR of vector pTRPE AVA L2H CAR was replaced with the sequence encoding the CAR of SEQ ID NO: X, and transduced into T cells to obtain anti-IGHV3-23 BCR CAR-T cells (“CART3-23”). Anti-CD19 CAR T cells (“CART19”) and untransduced T cells (“UTD”) served as positive and negative control respectively.

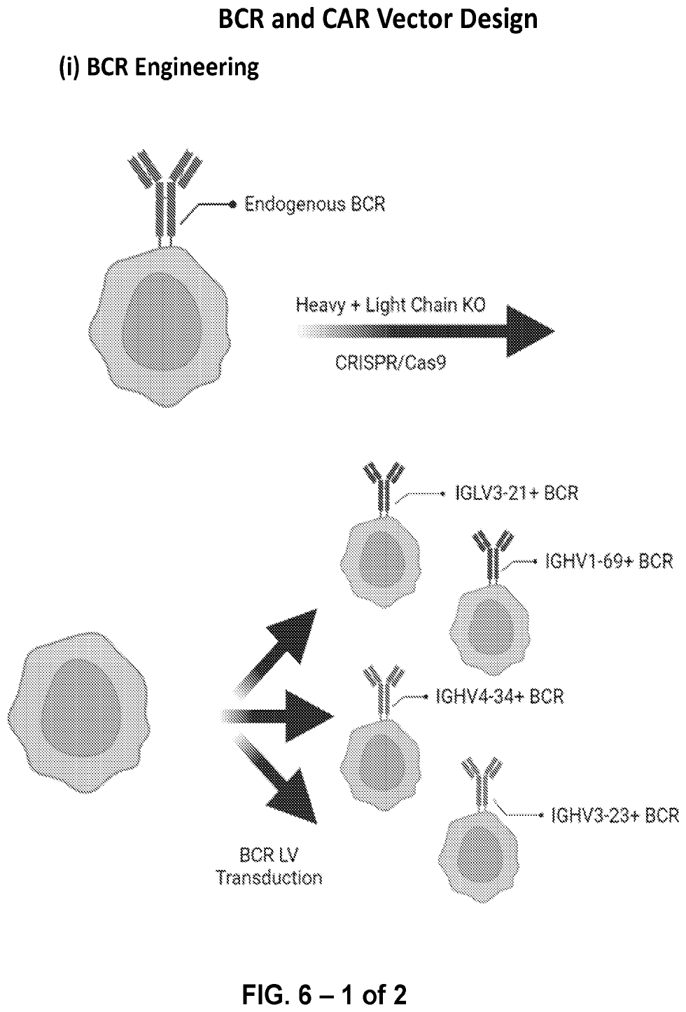

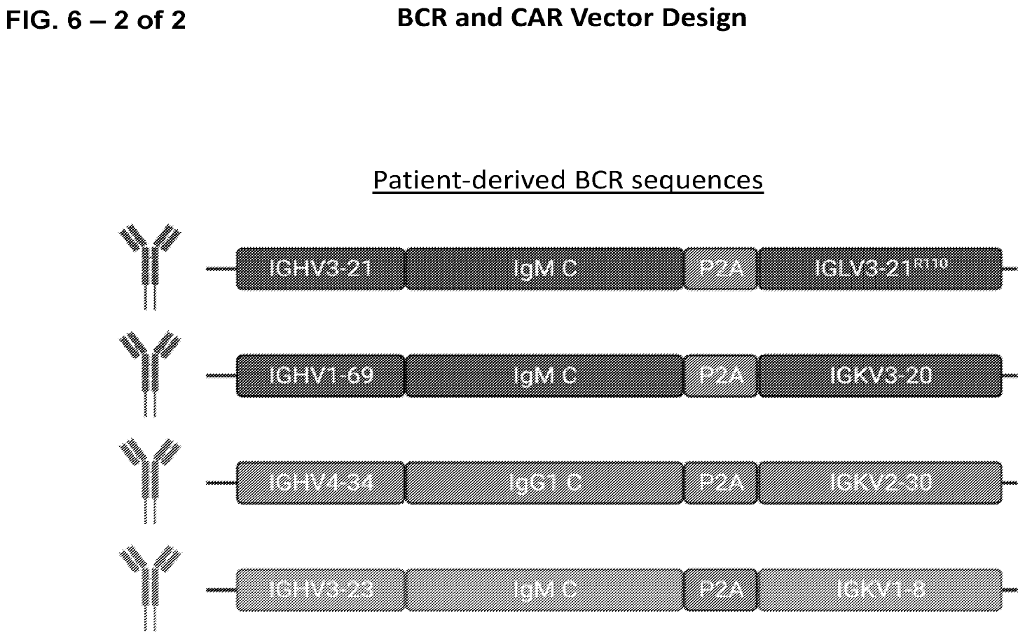

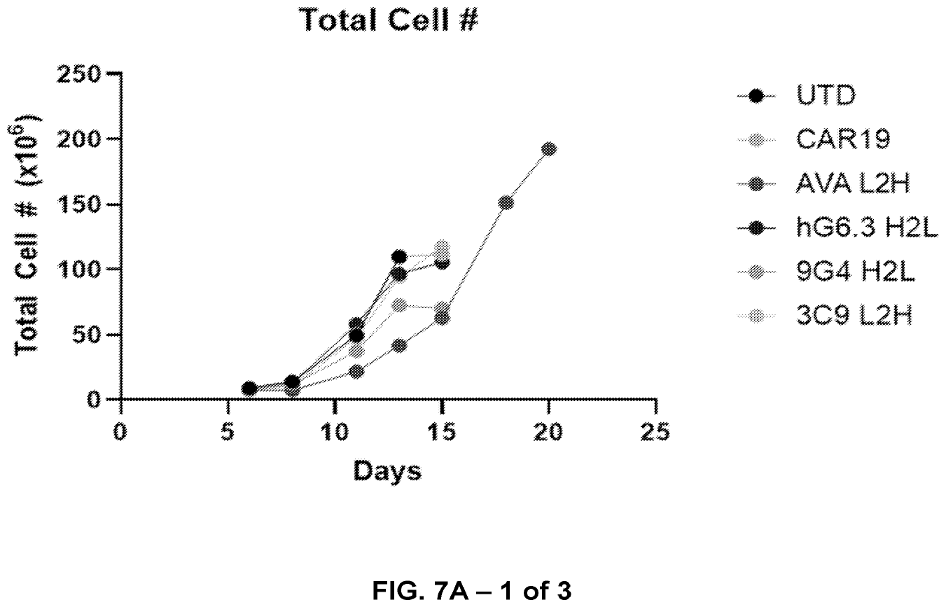

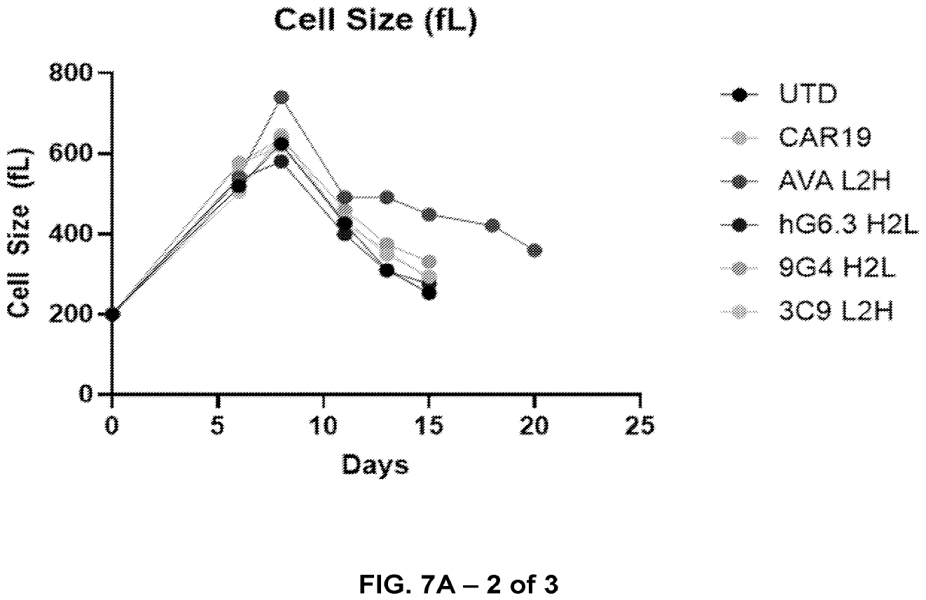

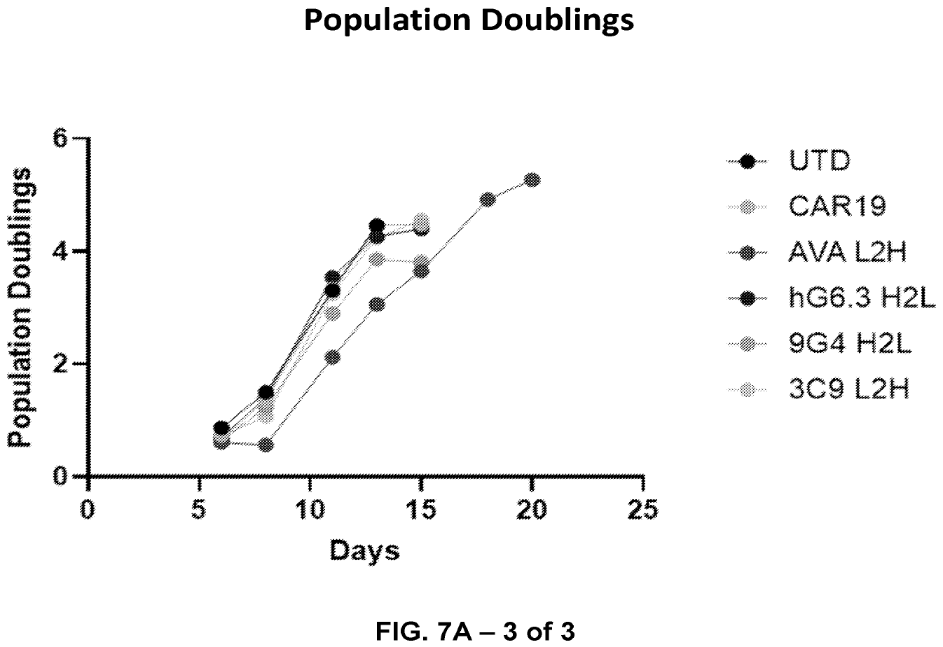

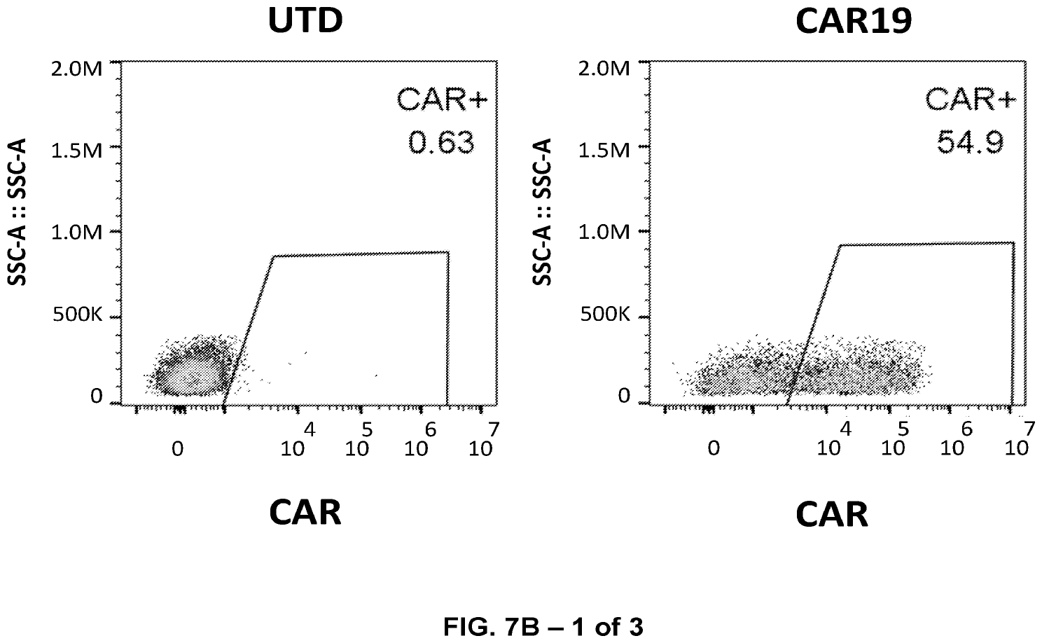

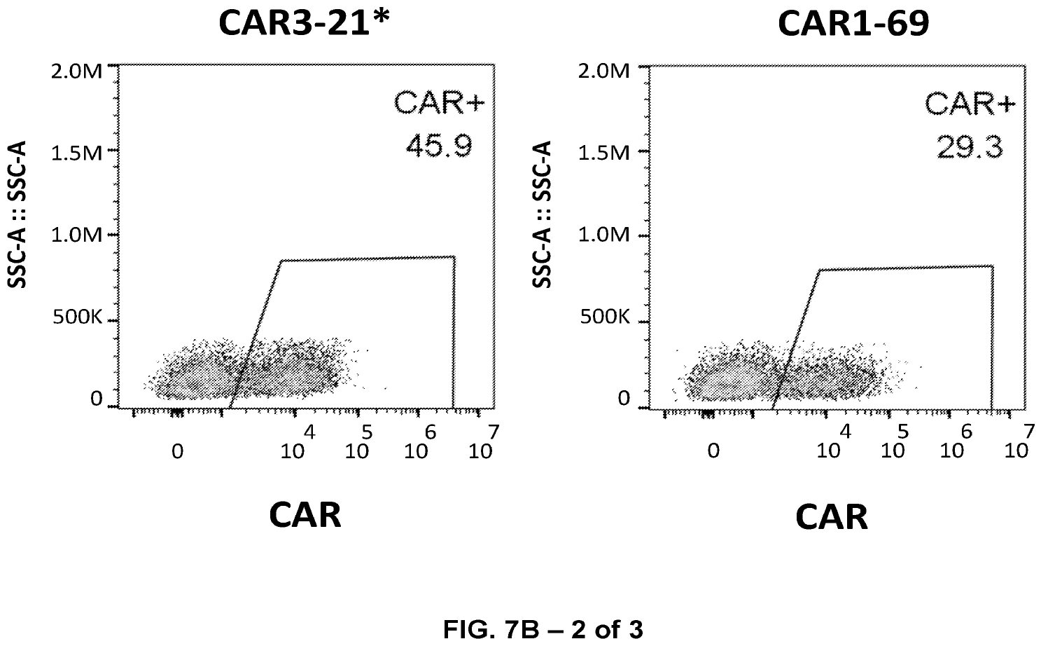

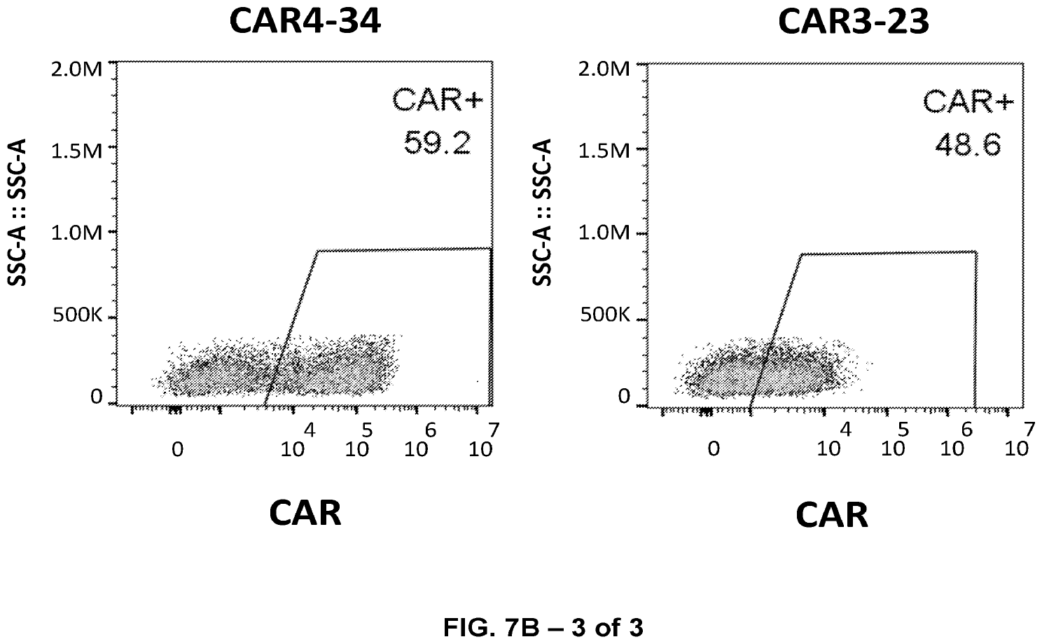

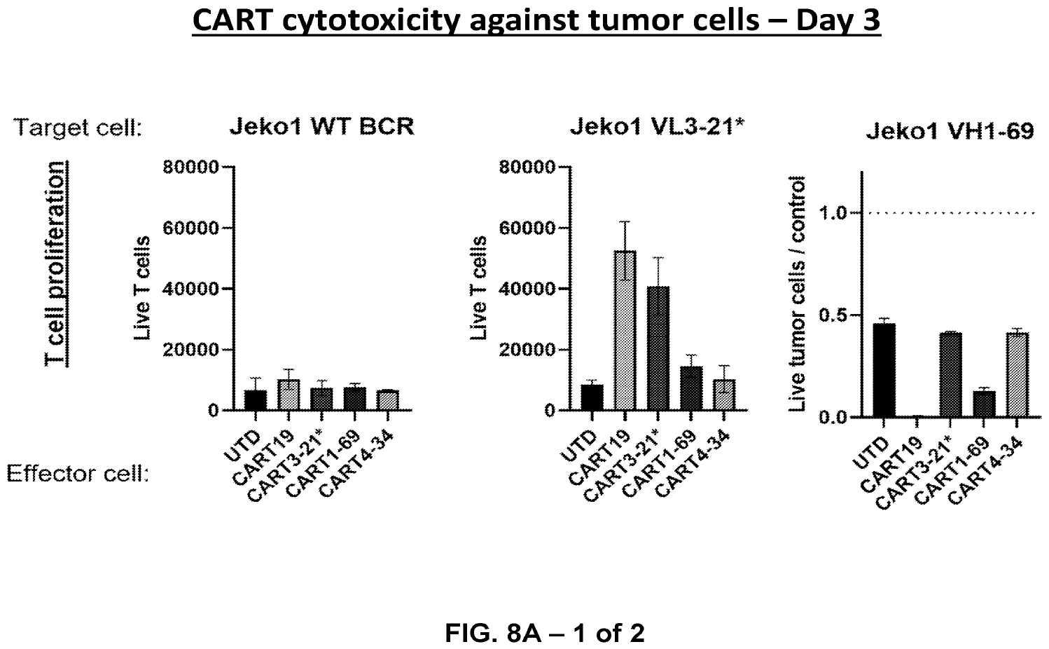

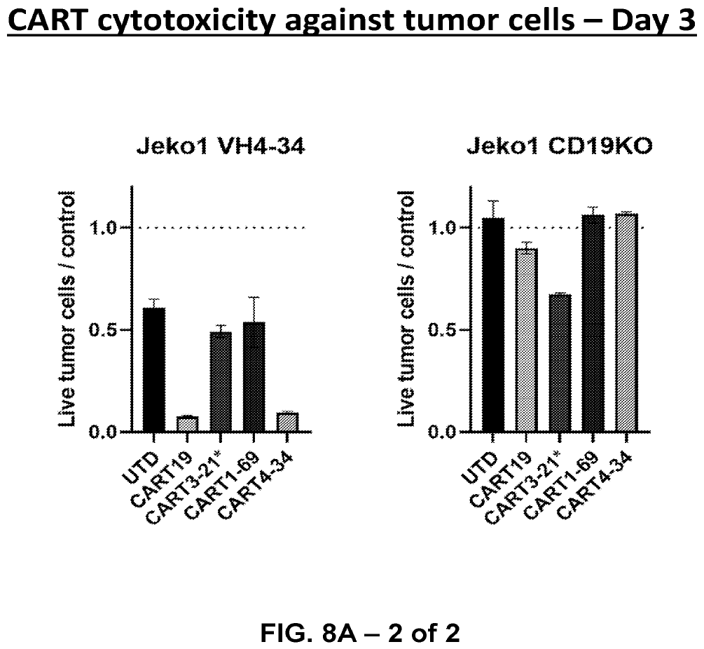

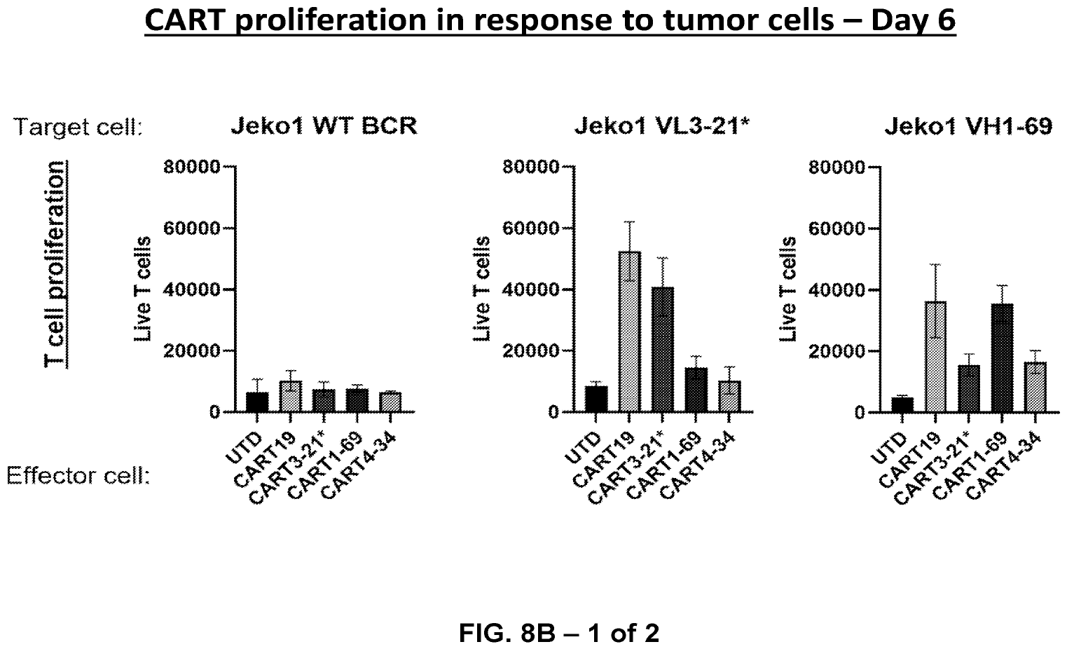

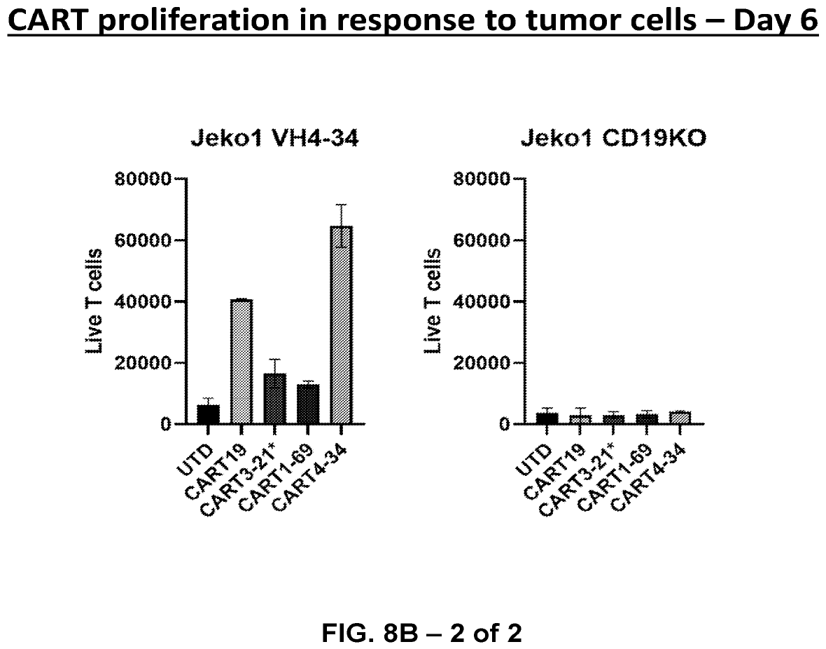

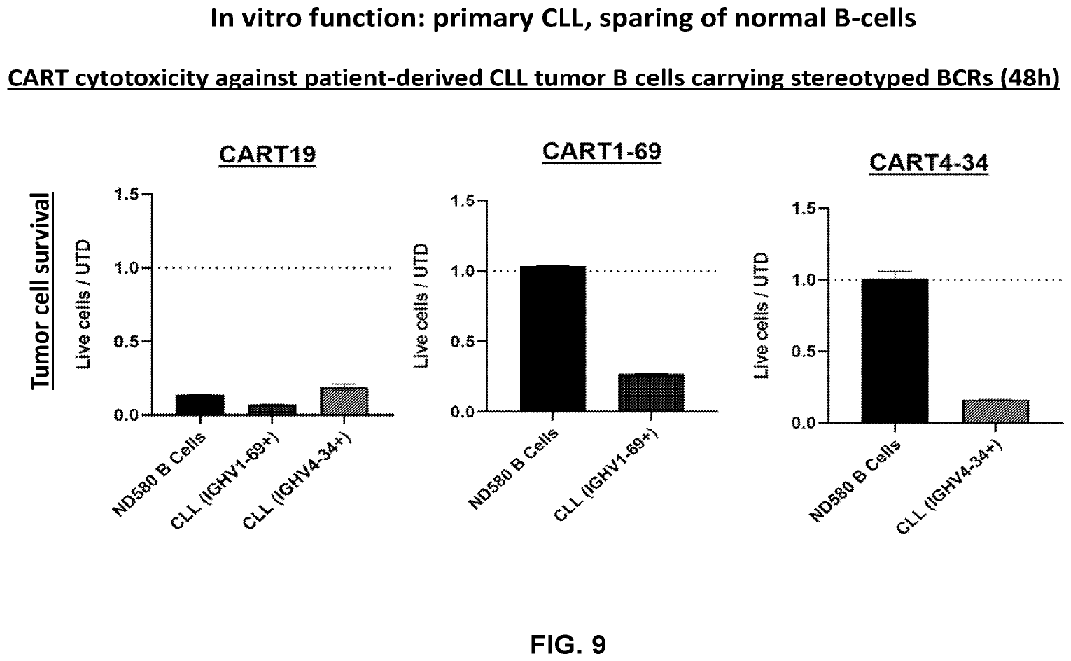

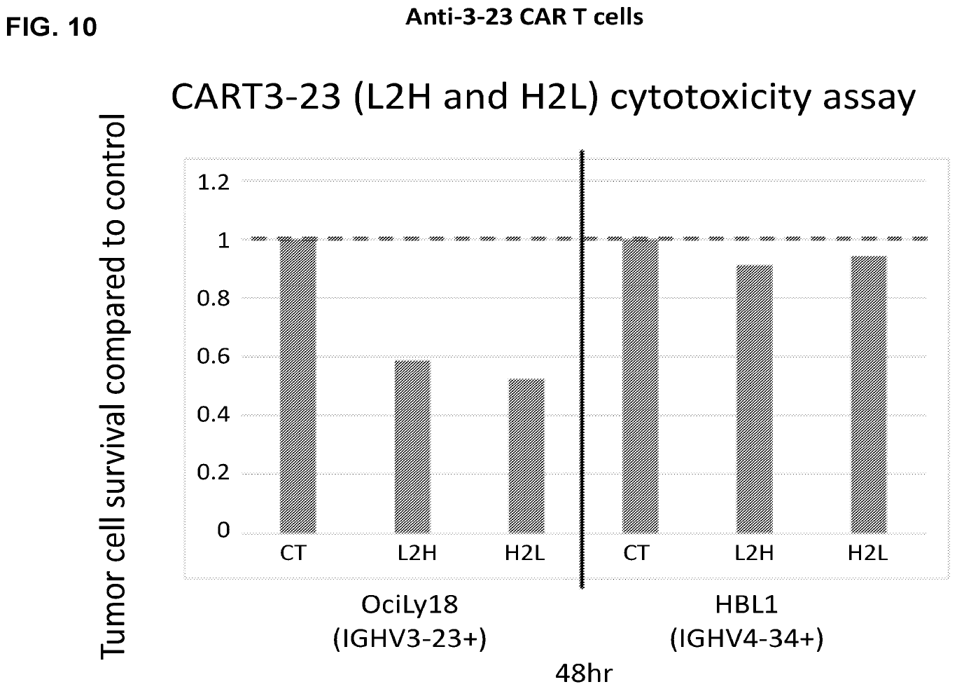

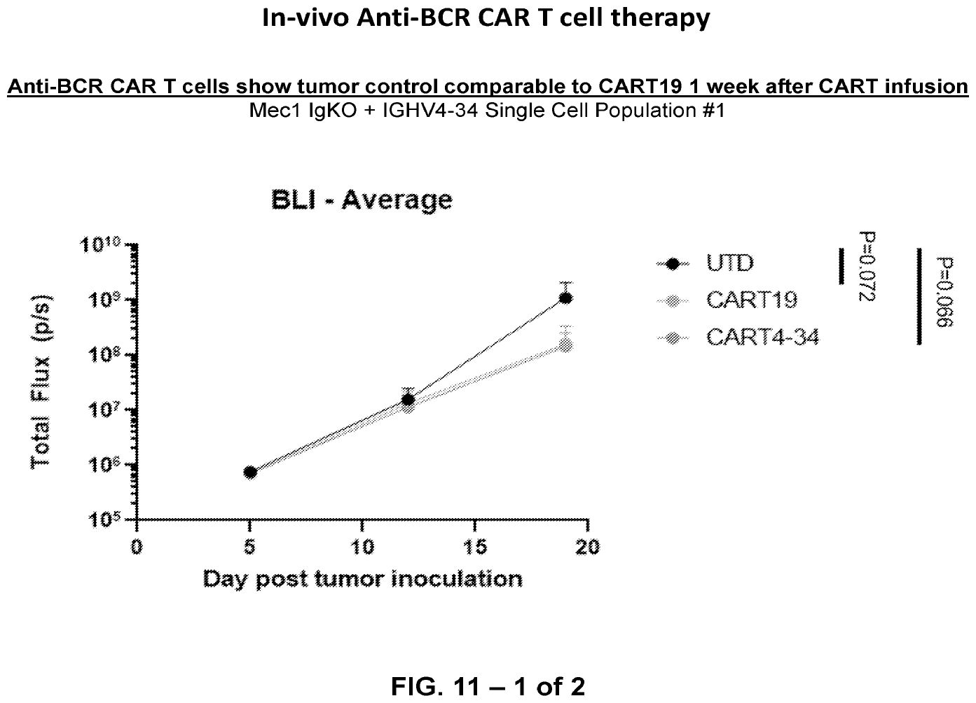

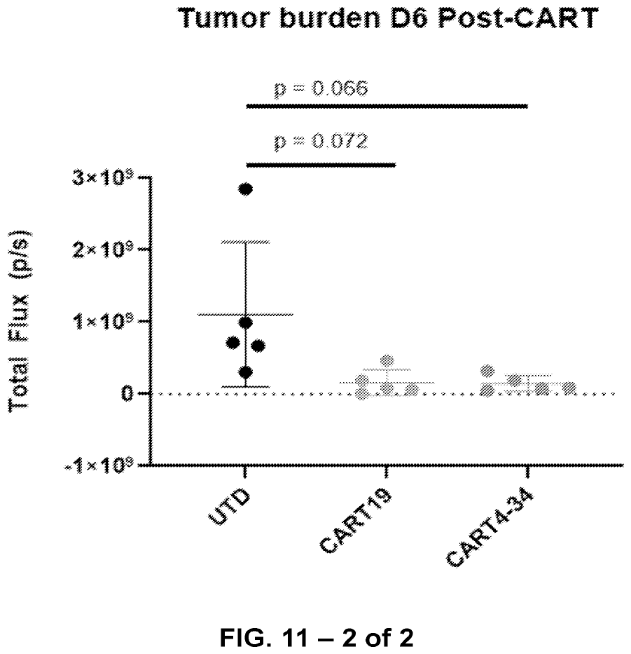

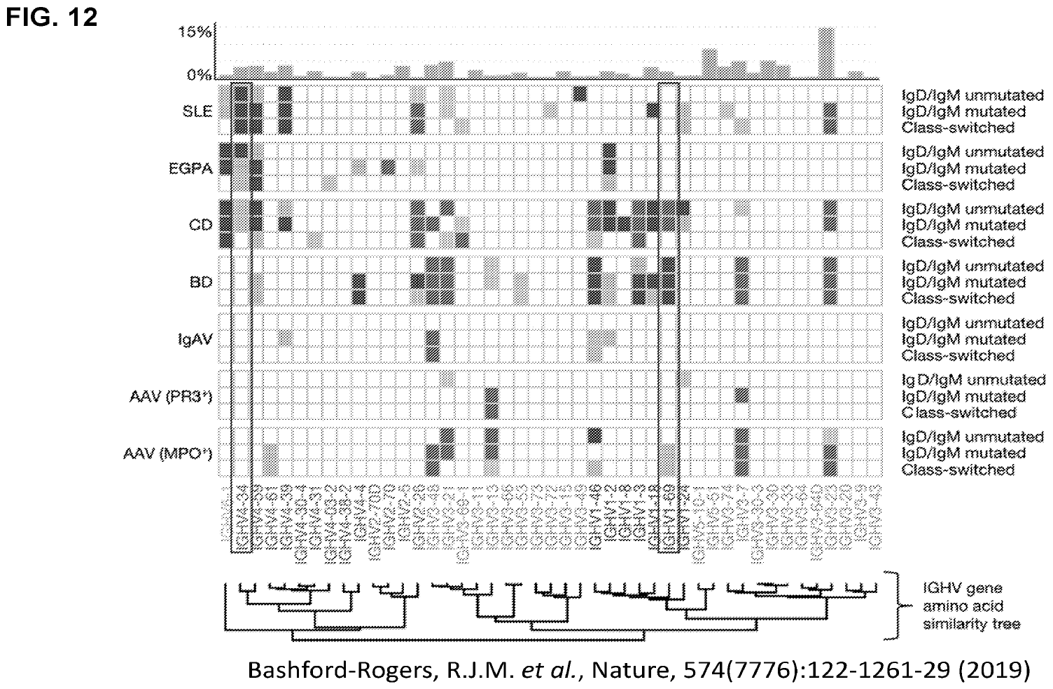

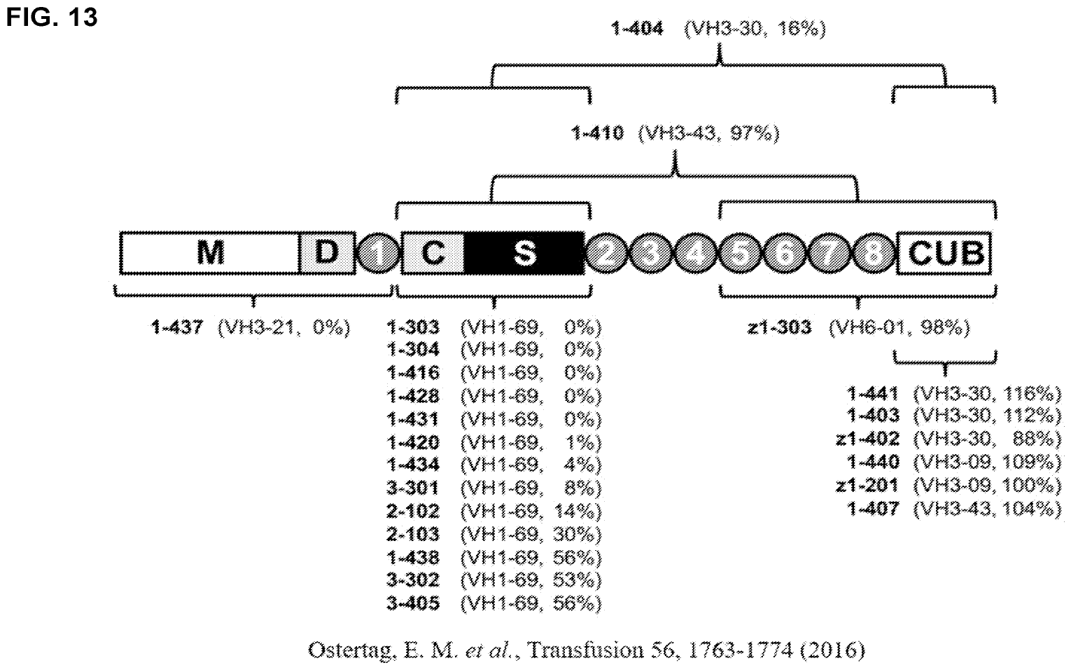

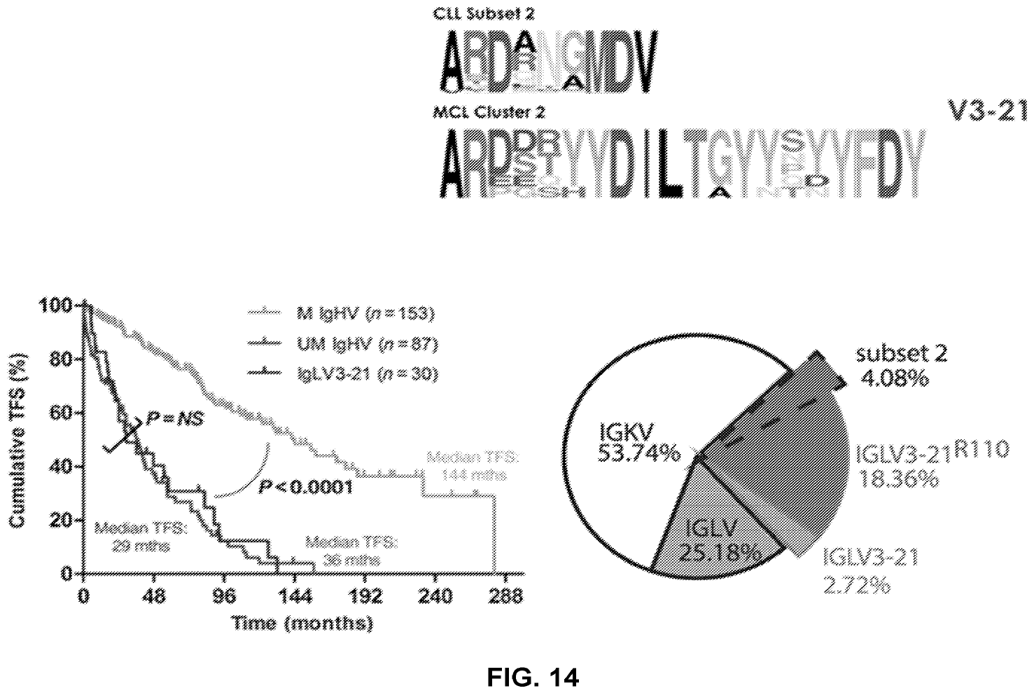

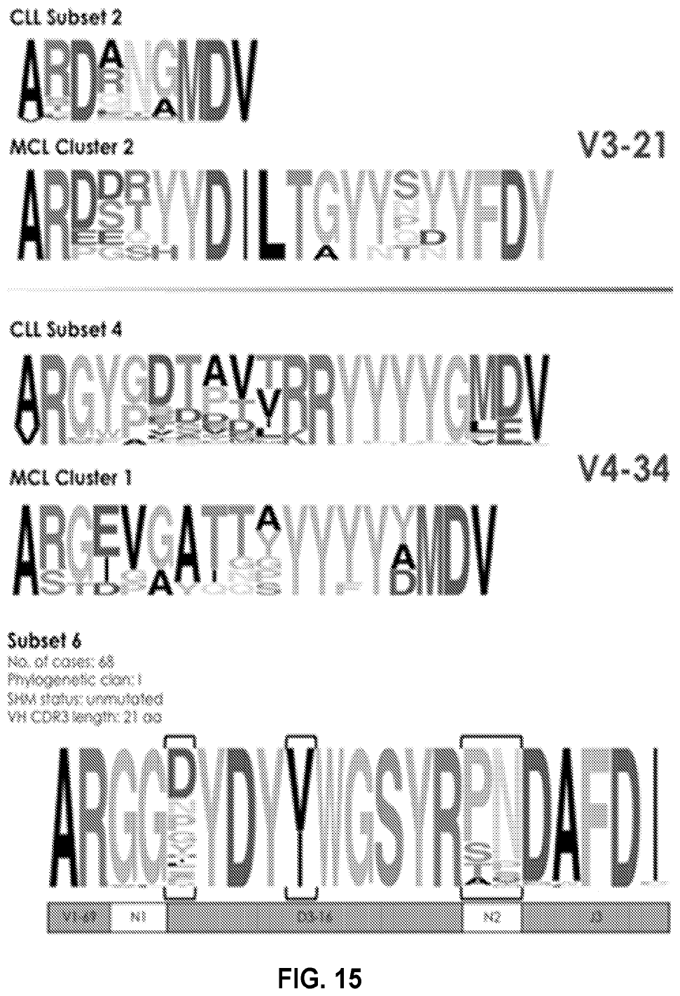

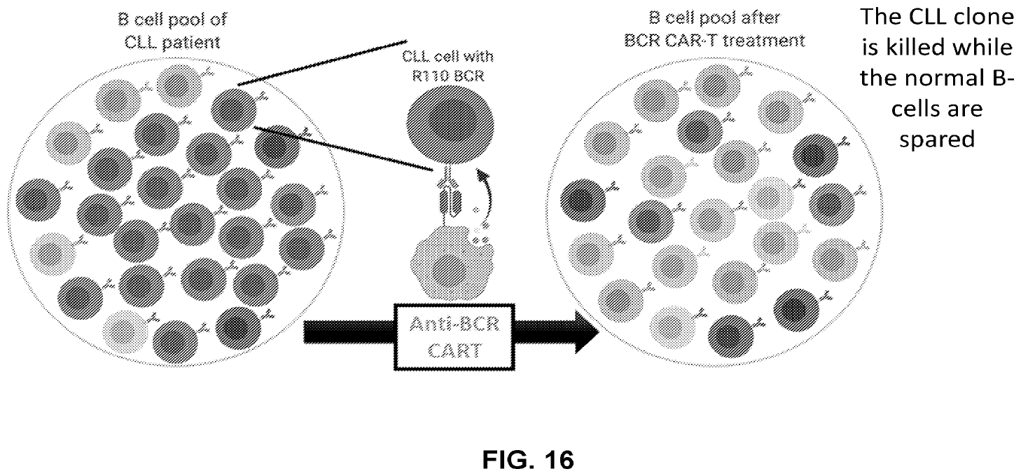

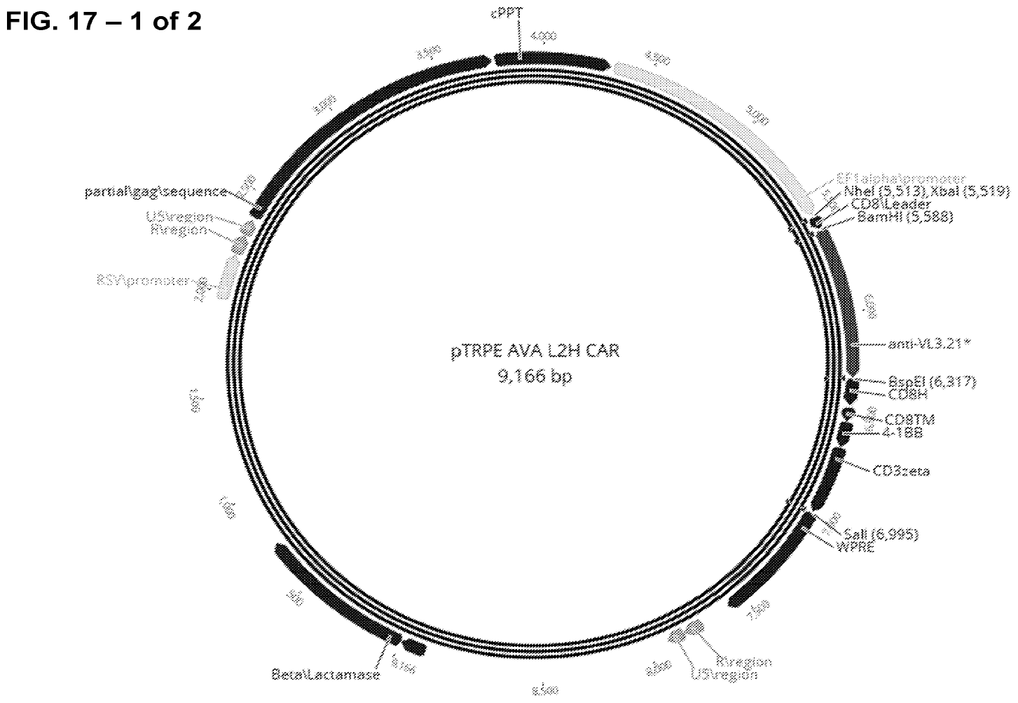

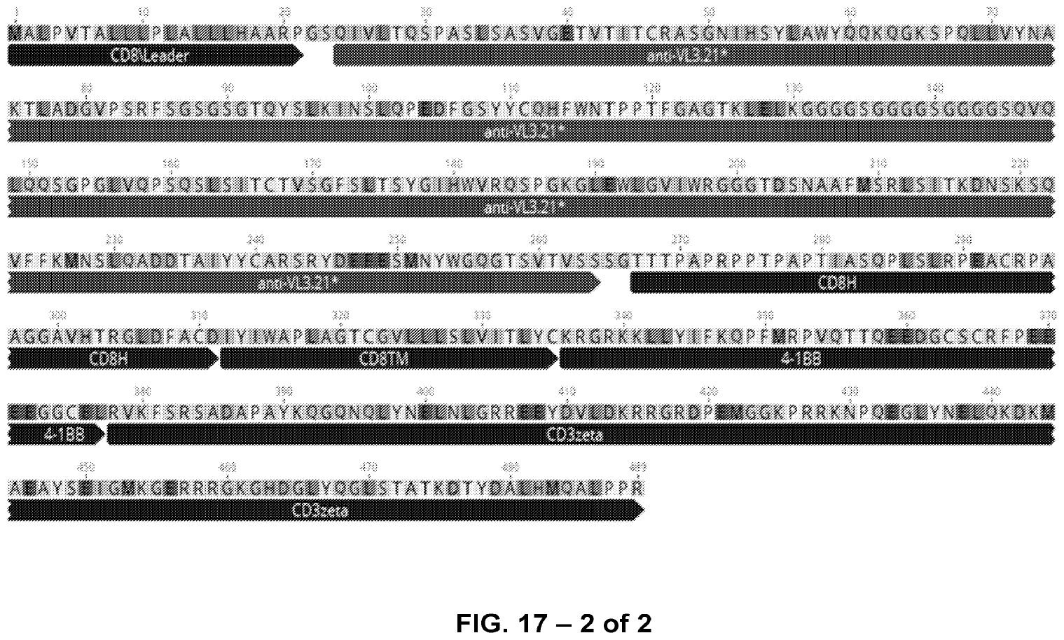

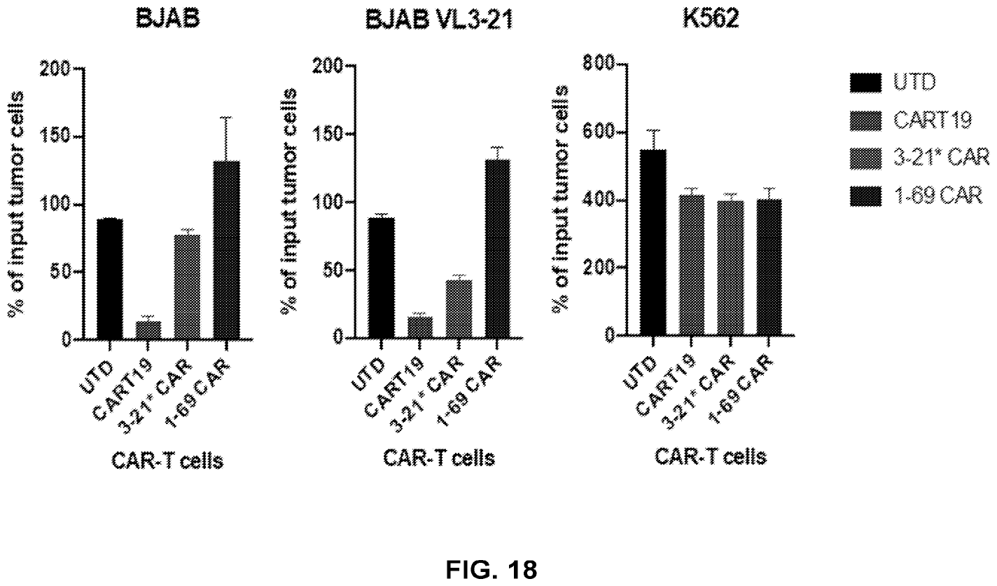

Cytotoxicity of each of the Jekol cells (Jekol WT BCR, Jekol VL3-21*, Jekol VH1-69, Jekol VH4-34, Jekol CD19KO) was determined following coculturing of cells with transduced or untransduced T cells, and is shown in FIG. 6. T cells transduced with anti-IGLV3-21 R110 BCR CAR-T cells (“CART3-21*”) specifically kill Jekol VL3-21* cells expressing VL3-21 R110 BCR while sparing other cells (FIG. 6, top graph), and CART3-21* T cells proliferate over time specifically in response to Jekol VL3-21 cells (FIG. 6, bottom graph). T cells transduced with anti-IGHV1-69 BCR CAR T cells (CART1-69) specifically kill Jekol VH1-69 cells expressing VH1-69 BCR while sparing other cells, and CART1-69 T cells proliferate over time specifically in response to Jekol VH1-69 cells; and T cells transduced with anti-IGHV4-34 BCR CAR T cells specifically kill Jekol VH4-34 cells expressing VH4-34 BCR while sparing other cells, and CART4-34 T cells proliferate over time specifically in response to Jekol VH4-34cells.

### Enumerated Embodiments

The following enumerated embodiments are provided, the numbering of which is not to be construed as designating levels of importance.

Embodiment 1 provides a chimeric antigen receptor (CAR) comprising an antigen binding domain, a transmembrane domain, and an intracellular signaling domain, wherein the antigen binding domain specifically binds to a B cell receptor (BCR).

Embodiment 2 provides the CAR of embodiment 1, wherein the antigen binding domain is an antibody or an antigen-binding fragment thereof.

Embodiment 3 provides the CAR of embodiment 1 or 2, wherein the antigen-binding fragment is a single-chain variable fragment (scFv), a fragment antigen-binding (Fab) or a single-domain antibody.

Embodiment 4 provides the CAR of any one of embodiments 1-3, wherein the antigen-binding fragment is a scFv.

Embodiment 5 provides the CAR of any one of embodiments 1-4, wherein the enriched stereotyped BCR is a membrane-bound protein on a B cell or plasma cell.

Embodiment 6 provides the CAR of any one of embodiments 1-5, wherein the enriched stereotyped BCR comprises the amino acid sequence set forth in any one of SEQ ID NOs: 13-17, 18-20, and 21-24; or an amino acid sequence having at least 85% identity to the sequence as set forth in any one of SEQ ID NOs: 13-17, 18-20, and 21-24.

Embodiment 7 provides the CAR of any one of embodiments 1-6, wherein the antigen binding domain comprises the amino acid sequence set forth in any one of SEQ ID NOs: 25-30, 31-33, 37-39, and 43-48; or an amino acid sequence having at least 85% identity to the sequence as set forth in any one of SEQ ID NOs: 25-30, 31-32, 35-36, 39-42, 64, 66, 68, 69, 76, 78, 80, and 81.

Embodiment 8 provides the CAR of any one of embodiments 1-7, wherein the transmembrane domain comprises a CD8 transmembrane domain.

Embodiment 9 provides the CAR of any one of embodiments 1-8, wherein the transmembrane domain comprises the amino sequence of SEQ ID NO: 51; or an amino acid sequence having at least 85% identity to the sequence set forth in SEQ ID NO: 51.

Embodiment 10 provides the CAR of any one of embodiments 1-9, wherein the intracellular signaling domain comprises a CD3 zeta signaling domain.

Embodiment 11 provides the CAR of any one of embodiments 1-10, wherein the intracellular signaling domain comprises the amino acid sequence of SEQ ID NO: 56; or an amino acid sequence having at least 85% identity to the sequence set forth in SEQ ID NO: 56.

Embodiment 12 provides the CAR of any one of embodiments 1-11, wherein the CAR further comprises an intracellular domain of a costimulatory molecule.

Embodiment 13 provides the CAR of embodiment 12, wherein the costimulatory molecule is 4-1BB.

Embodiment 14 provides the CAR of embodiment 12 or 13, wherein the intracellular domain comprises the amino acid sequence of SEQ ID NO: 59; or an amino acid sequence having at least 85% identity to the sequence set forth in SEQ ID NO: 59.

Embodiment 15 provides the CAR of any one of embodiments 1-14, wherein the CAR further comprises a CD8 alpha hinge.

Embodiment 16 provides the CAR of embodiment 15, wherein the CD8 alpha hinge comprises the amino acid sequence of SEQ ID NO: 54; or an amino acid sequence having at least 85% identity to the sequence set forth in SEQ ID NO: 54.

Embodiment 17 provides the CAR of any one of embodiments 1-16, wherein the CAR further comprises a CD8 signal peptide.

Embodiment 18 provides the CAR of embodiment 17, wherein the CD8 signal peptide comprises the amino acid sequence of SEQ ID NO: 63; or an amino acid sequence having at least 85% identity to the sequence set forth in SEQ ID NO: 63.

Embodiment 19 provides the CAR of any one of embodiments 1-14, wherein CAR comprises the amino acid sequence set forth in any one of SEQ ID NOs: 47-50; or an amino acid sequence having at least 85% identity to the sequence set forth in any one of SEQ ID NOs: 47-50 and 72-75.

Embodiment 20 provides a nucleic acid molecule comprising a nucleic acid sequence encoding the CAR of any one of embodiments 1-19.

Embodiment 21 provides a vector comprising the nucleic acid of embodiment 20.

Embodiment 22 provides a genetically modified cell comprising the CAR of any one of embodiments 1-21.

Embodiment 23 provides the genetically modified cell of embodiment 22, wherein the cell is a human T cell.

Embodiment 24 provides a pharmaceutical composition comprising the CAR of any one of embodiments 1-19, the nucleic acid molecule of embodiment 20, the vector of embodiment 21, or the genetically modified cell of embodiment 22 or 23; and a pharmaceutically acceptable excipient.

Embodiment 25 provides a method for specifically eliminating a enriched stereotyped B cell in a subject in need thereof, the method comprising administering to the subject an effective amount of the genetically modified cell of embodiment 23 or 23.

Embodiment 26 provides a method of for treating or preventing a hematologic cancer in a subject in need thereof, the method comprising administering to the subject an effective amount of the genetically modified cell of embodiment 22 or 23.

Embodiment 27 provides the method of embodiment 26, wherein the hematologic cancer is a leukemia.

Embodiment 28 provides the method of embodiment 26 or 27, wherein the hematologic cancer is chronic lymphocytic leukemia, mantle cell lymphoma, diffuse large B-cell lymphoma, splenic marginal zone lymphoma, acute lymphocytic leukemia, acute myelocytic leukemia, acute myelogenous leukemia, chronic myelocytic (granulocytic) leukemia, chronic myelogenous leukemia, or chronic lymphocytic leukemia.

Embodiment 29 provides a method of for treating or preventing an autoimmune disease in a subject in need thereof, the method comprising administering to the subject an effective amount of the genetically modified cell of embodiment 22 or 23.

Embodiment 30 provides the method of embodiment 29, wherein the autoimmune disease is systemic lupus erythematosus (SLE), Crohn's disease (CD), Behcet's disease (BD), eosinophilic granulomatosis with polyangiitis (EGPA), thrombotic thrombocytopenia purpura (TTP), ANCA-associated vasculitis (AAV), IgA vasculitis (IgAV), or IgA vasculitis (IgAV).

Embodiment 31 provides the method of any one of embodiments 25-30, wherein the subject is a human.

Embodiment 32 provides use of the genetically modified cell of embodiment 22 or 23 for the manufacture of a medicament for the treatment or prevention of a hematologic cancer or an autoimmune disease in a subject in need thereof.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A chimeric antigen receptor (CAR) comprising an antigen binding domain, a transmembrane domain, and an intracellular signaling domain, wherein the antigen binding domain specifically binds to a disease-specific or enriched stereotyped B cell receptor (BCR).

2. The CAR of claim 1, wherein the antigen binding domain is an antibody or an antigen-binding fragment thereof.

3. The CAR of claim 1, wherein the antigen-binding fragment is a single-chain variable fragment (scFv), a fragment antigen-binding (Fab) or a single-domain antibody.

4. The CAR of claim 1, wherein the antigen-binding fragment is a scFv.

5. The CAR of claim 1, wherein the enriched stereotyped BCR is a membrane-bound protein on a B cell or plasma cell.

6. The CAR of claim 1, wherein the enriched stereotyped BCR comprises the amino acid sequence set forth in any one of SEQ ID NOs: 13-17, 18-20, and 21-24; or an amino acid sequence having at least 85% identity to the sequence as set forth in any one of SEQ ID NOs: 13-17, 18-20, and 21-24.

7. The CAR of claim 1, wherein the antigen binding domain comprises the amino acid sequence set forth in any one of SEQ ID NOs: 25-30, 31-32, 35-36, 39-42, 64, 66, 68, and 69; or an amino acid sequence having at least 85% identity to the sequence as set forth in any one of SEQ ID NOs: 25-30, 31-32, 35-36, 39-42, 64, 66, 68, 69, 76, 78, 80, and 81.

8. The CAR of claim 1, wherein the transmembrane domain comprises a CD8 transmembrane domain.

9. The CAR of claim 1, wherein the transmembrane domain comprises the amino sequence of SEQ ID NO: 51; or an amino acid sequence having at least 85% identity to the sequence set forth in SEQ ID NO: 51.

10. The CAR of claim 1, wherein the intracellular signaling domain comprises a CD3 zeta signaling domain.

11. The CAR of claim 1, wherein the intracellular signaling domain comprises the amino acid sequence of SEQ ID NO: 56; or an amino acid sequence having at least 85% identity to the sequence set forth in SEQ ID NO: 56.

12. The CAR of claim 1, wherein the CAR further comprises an intracellular domain of a costimulatory molecule.

13. The CAR of claim 12, wherein the costimulatory molecule is 4-1BB.

14. The CAR of claim 12, wherein the intracellular domain comprises the amino acid sequence of SEQ ID NO: 59; or an amino acid sequence having at least 85% identity to the sequence set forth in SEQ ID NO: 59.

15. The CAR of claim 1, wherein the CAR further comprises a CD8 alpha hinge.

16. The CAR of claim 15, wherein the CD8 alpha hinge comprises the amino acid sequence of SEQ ID NO: 54; or an amino acid sequence having at least 85% identity to the sequence set forth in SEQ ID NO: 54.

17. The CAR of claim 1, wherein the CAR further comprises a CD8 signal peptide.

18. The CAR of claim 17, wherein the CD8 signal peptide comprises the amino acid sequence of SEQ ID NO: 63; or an amino acid sequence having at least 85% identity to the sequence set forth in SEQ ID NO: 63.

19. The CAR of claim 1, wherein CAR comprises the amino acid sequence set forth in any one of SEQ ID NOs: 47-50; or an amino acid sequence having at least 85% identity to the sequence set forth in any one of SEQ ID NOs: 47-50 and 72-75.

20. A nucleic acid molecule comprising a nucleic acid sequence encoding the CAR of claim 1.

21. A vector comprising the nucleic acid of claim 20.

22. A genetically modified cell comprising the CAR of claim 1.

23. The genetically modified cell of claim 22, wherein the cell is a human T cell.

24. A pharmaceutical composition comprising the nucleic acid molecule of claim 20; and a pharmaceutically acceptable excipient.

25. A method for specifically eliminating a enriched stereotyped B cell in a subject in need thereof, the method comprising administering to the subject an effective amount of the genetically modified cell of claim 23.

26. A method of for treating or preventing a hematologic cancer in a subject in need thereof, the method comprising administering to the subject an effective amount of the genetically modified cell of claim 22.

27. The method of claim 26, wherein the hematologic cancer is a leukemia.

28. The method of claim 26, wherein the hematologic cancer is chronic lymphocytic leukemia, mantle cell lymphoma, diffuse large B-cell lymphoma, splenic marginal zone lymphoma, acute lymphocytic leukemia, multiple myeloma, acute myelocytic leukemia, acute myelogenous leukemia, chronic myelocytic (granulocytic) leukemia, chronic myelogenous leukemia, or chronic lymphocytic leukemia.

29. A method of for treating or preventing an autoimmune disease in a subject in need thereof, the method comprising administering to the subject an effective amount of the genetically modified cell of claim 22.

30. The method of claim 29, wherein the autoimmune disease is systemic lupus erythematosus (SLE), Crohn's disease (CD), Behcet's disease (BD), eosinophilic granulomatosis with polyangiitis (EGPA), thrombotic thrombocytopenia purpura (TTP), ANCA-associated vasculitis (AAV), IgA vasculitis (IgAV), or IgA vasculitis (IgAV).

31. The method of claim 25, wherein the subject is a human.

32. (canceled)
